(19) United States
(12) Patent Application Publication
(10) Pub. No.: US 2025/0276982 A1
(43) Pub. Date: Sep. 4, 2025

(54) KRAS INHIBITORS

(71) Applicants: ASCENTAGE PHARMA (SUZHOU) CO., LTD., Suzhou (CN); ASCENTAGE PHARMA GROUP CORP LIMITED, Central (HK)

(72) Inventors: Chao LI, Suzhou (CN); Jianyong CHEN, Suzhou (CN); Gaojie YE, Suzhou (CN); Liugen LI, Suzhou (CN); Wenliang HU, Suzhou (CN); Chengzhe WU, Suzhou (CN)

(21) Appl. No.: 18/293,649

(22) PCT Filed: Sep. 2, 2022
(86) PCT No.: PCT/CN2022/116807

§ 371 Date: Jan. 30, 2024

(30) Foreign Application Priority Data
Sep. 3, 2021 (WO) PCT/CN2021/116521

(51) Int. Cl. C07D 519/00; A61K 31/517; A61K 31/519; A61K 31/5377; A61K 31/5386; A61P 35/00; C07D 487/08

(52) U.S. Cl. CPC C07D 519/00; A61K 31/517; A61K 31/519; A61K 31/5377; A61K 31/5386; A61P 35/00; C07D 487/08

(57) ABSTRACT

The present disclosure relates to compounds that inhibit KRAS. In particular, the present disclosure relates to compounds that inhibit the activity of KRAS G12D, pharmaceutical compositions comprising the compounds and methods of use therefor.

### CROSS REFERENCE TO RELATED APPLICATIONS

This application is a National Stage application of International Application No. PCT/CN2022/116807, filed 2 Sep. 2022. This application also claims priority under 35 U.S.C. § 119 to International Patent Application No. PCT/CN2021/116521, filed 3 Sep. 2021.

### TECHNICAL FIELD

The present disclosure relates to compounds that inhibit KRAS. In particular, the present disclosure relates to compounds that inhibit the activity of KRAS G12D, pharmaceutical compositions comprising the compounds and methods of use therefor.

### BACKGROUND OF THE INVENTION

RAS represents a group of monomeric globular proteins of 189 amino acids (21 kDa molecular mass) that are associated with the plasma membrane and that bind either GDP or GTP. RAS acts as a molecular switch. When RAS contains bound GDP, it is in the resting or off position and is “inactive”. In response to exposure of the cell to certain growth promoting stimuli, RAS is induced to exchange its bound GDP for a GTP. With GTP bound, RAS is “switched on” and is able to interact with and activate other proteins (its “downstream targets”). The RAS protein itself has a very low intrinsic ability to hydrolyze GTP back to GDP, thus turning itself into the off state. Switching RAS off requires extrinsic proteins termed GTPase-activating proteins (GAPs) that interact with RAS and greatly accelerate the conversion of GTP to GDP. Any mutation in RAS that affects its ability to interact with GAP or to convert GTP back to GDP will result in a prolonged activation of the protein and consequently a prolonged signal to the cell telling it to continue to grow and divide. Because these signals result in cell growth and division, overactive RAS signaling may ultimately lead to cancer. The most notable members of the RAS are HRAS, KRAS, and NRAS.

Structurally, RAS proteins contain a G domain that is responsible for the enzymatic activity of RAS, i.e., the guanine nucleotide binding and the hydrolysis (GTPase reaction). It also contains a C-terminal extension, known as the CAAX box, which may be post-translationally modified, and is responsible for targeting the protein to the membrane. The G domain is approximately 21-25 kDa in size and it contains a phosphate binding loop (P-loop). The P-loop represents the pocket where the nucleotides are bound in the protein, and this is the rigid part of the domain with conserved amino acid residues that are essential for nucleotide binding and hydrolysis (Glycine 12, Threonine 26 and Lysine 16). The G domain also contains the Switch I (residues 30-40) and Switch II (residues 60-76) regions, both of which are the dynamic parts of the protein which are often represented as the “spring-loaded” mechanism because of their ability to switch between the resting and loaded state. The key interaction is the hydrogen bonds formed by Threonine-35 and glycine-60 with the gamma-phosphate of GTP that maintain the Switch 1 and Switch 2 regions, respectively, in their active conformation. After hydrolysis of GTP and release of phosphate, these two relax into the inactive GDP conformation.

Mutations in the KRAS gene are common events in human tumorigenesis. Indeed, mutations in KRAS are prevalent in the some of the most deadly cancer types: pancreatic (95%), colorectal (45%), and lung (35%). The most common KRAS mutations are found at residue G12 and G13 in the P-loop and at residue Q61.

Single nucleotide substitutions that result in missense mutations at codons 12 and 13 of the KRAS primary amino acid sequence comprise approximately 40% of these KRAS driver mutations in lung adenocarcinoma. KRAS G12D mutation is present in 25.0% of all pancreatic ductal adenocarcinoma patients, 13.3% of all colorectal carcinoma patients, 10.10% of all rectal carcinoma patients, 4.10% of all non-small cell lung carcinoma patients and 1.7% of all small cell lung carcinoma patients (e.g., see The AACR Project GENIE Consortium, (2017) Cancer Discovery; 7 (8):818-831. Dataset Version 4).

Compounds that inhibit KRAS activity are still highly desirable and under investigation, including those that disrupt effectors such as guanine nucleotide exchange factors (e.g., see Sun et al., (2012) Agnew Chem Int Ed Engl. 51 (25): 6140-6143 doi: 10.1002/anie201201358) as well recent advances in the covalent targeting of an allosteric pocket of KRAS G12C (e.g., see Ostrem et al., (2013) Nature 503:548-551 and Fell et al., (2018) ACS Med. Chem. Lett. 9:1230-1234). Clearly there remains a continued interest and effort to develop inhibitors of KRAS, particularly inhibitors of activating KRAS mutants, especially KRAS G12D.

Thus, there is a need to develop new KRAS (especially KRAS G12D) inhibitors that demonstrate sufficient efficacy for treating KRAS G12D-mediated cancer.

### SUMMARY OF THE INVENTION

In one aspect, the present disclosure provides compounds represented by any one of Formulae I, Ia, Ib, II, IIa and IIb below, and the pharmaceutically acceptable salts and solvates, e.g., hydrates, thereof, collectively referred to as “compounds of the disclosure.” Compounds of the disclosure are KRAS (especially KRAS G12D) inhibitors and are thus useful in treating or preventing diseases or conditions such as KRAS G12D-associated cancer wherein the inhibition of KRAS G12D provides a benefit.

In another aspect, the present disclosure provides pharmaceutical compositions comprising the compound of the disclosure, and a pharmaceutically acceptable carrier.

In another aspect, the present disclosure provides methods for inhibiting KRAS G12D activity in a cell, comprising contacting the cell in which inhibition of KRAS G12D activity is desired with an effective amount of a compound of the disclosure; or a pharmaceutical composition of the disclosure.

In another aspect, the present disclosure provides methods for treating a KRAS G12D-associated cancer comprising administering to a patient in need thereof a therapeutically effective amount of a compound of the disclosure; or a pharmaceutical composition of the disclosure. Preferably, the cancer is non-small cell lung cancer, small cell lung cancer, colorectal cancer, rectal cancer or pancreatic cancer.

In another aspect, the present disclosure provides a method for treating cancer in a patient in need thereof, the method comprising (a) determining that the cancer is associated with a KRAS G12D mutation (e.g., a KRAS G12D-associated cancer); and (b) administering to the patient a therapeutically effective amount of a compound of the disclosure; or a pharmaceutical composition of the disclosure.

In another aspect, the present disclosure provides compounds of the disclosure for use in inhibiting KRAS G12D activity in a cell.

In another aspect, the present disclosure provides compounds of the disclosure for use in treating a KRAS G12D-associated cancer.

In another aspect, the present disclosure provides the use of compounds of the disclosure in the manufacture of a medicament for inhibiting KRAS G12D activity in a cell.

In another aspect, the present disclosure provides the use of compounds of the disclosure in the manufacture of a medicament for treating a KRAS G12D-associated cancer.

In another aspect, the present disclosure provides kits comprising compounds of the disclosure, and instructions for administering the compounds of the disclosure to a subject (e.g., a patient) having cancer (e.g., KRAS G12D-associated cancer).

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF THE INVENTION

The present disclosure relates to compounds that inhibit KRAS. In particular, the present disclosure relates to compounds that inhibit the activity of KRAS G12D, pharmaceutical compositions comprising the compounds and methods of use therefor.

Unless otherwise defined below, all technical and scientific terms used herein have the same meanings as commonly understood by one of ordinary skill in the art. References to techniques used herein are intended to refer to techniques that are generally understood in the art, including those obvious changes or equivalent replacements of the techniques for those skilled in the art. While it is believed that the following terms are well understood by those skilled in the art, the following definitions are set forth to better explain the disclosure.

### I. Definitions

As used herein, the terms “including”, “comprising”, “having”, “containing” or “comprising”, and other variants thereof, are inclusive or open, and do not exclude other unlisted elements or method steps.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “KRAS” as used herein refers collectively to the wild-type KRAS gene and protein, and mutant forms thereof. The mutations found most frequently in the KRAS gene are primarily at codons 12, 13, or 61. KRAS mutations also occur in codons 63, 117, 119, and 146. Liu et al., Acta Pharmaceutica Sinica B 9:871-879 (2019).

The term “KRAS inhibitor” as used herein refers a compound that inhibits wild-type KRAS and/or mutant KRAS, and includes electrophilic compounds that form irreversible covalent bonds with the KRAS protein.

As used herein, “KRAS G12D” refers to a mutant form of a mammalian KRAS protein that contains an amino acid substitution of an aspartic acid for a glycine at amino acid position 12. The assignment of amino acid codon and residue positions for human KRAS is based on the amino acid sequence identified by UniProtKB/Swiss-Prot P01116: Variantp.Gly12Asp.

As used herein, a “KRAS G12D inhibitor” refers to compounds of the present disclosure that are represented by Formulae as described herein. These compounds are capable of negatively modulating or inhibiting all or a portion of the enzymatic activity of KRAS G12D.

A “KRAS G12D-associated disease or disorder” as used herein refers to diseases or disorders associated with or mediated by or having a KRAS G12D mutation. Anon-limiting example of a KRAS G12D-associated disease or disorder is a KRAS G12D-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRAS G12D mutation. In some embodiments, the subject has a tumor that is positive for a KRAS G12D mutation. The subject can be a subject with a tumor(s) that is positive for a KRAS G12D mutation. The subject can be a subject whose tumors have a KRAS G12D mutation. In some embodiments, the subject is suspected of having a KRAS G12D gene-associated cancer. In some embodiments, the subject has a clinical record indicating that the subject has a tumor that has a KRAS G12D mutation.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a compound of the disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is (are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “halo” or “halogen” as used herein by itself or as part of another group refers to —Cl, —F, —Br, or —I.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl. In another embodiment, one or more of the hydrogen atoms of the alkyl group are replaced by deuterium atoms, i.e., the alkyl group is isotopically-labeled with deuterium. A non-limiting exemplary deuterated alkyl group is —CD₃. In another embodiment, none of the hydrogen atoms of the alkyl group are replaced by deuterium atoms, i.e., the alkyl group is isotopically-labeled with deuterium.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl.

In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxy)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group and resulting alkoxy is thus referred to as a C₁-C₄ alkoxy. Non-limiting exemplary alkoxy groups include methoxy, ethoxy, and tert-butoxy.

The term “carbocyclic” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ carbocyclic. For example, a C₅ carbocyclic or a C₆ carbocyclic. When the aliphatic hydrocarbons are saturated, carbocyclic may also be called as cycloalkyl, e.g., a C₃ cycloalkyl such a cyclopropyl, a C₄ cycloalkyl such as cyclobutyl, etc. In one embodiment, the carbocyclic is cycloalkyl. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms, for example 5-membered, 6-membered, 9-membered, 10-membered heterocyclo. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

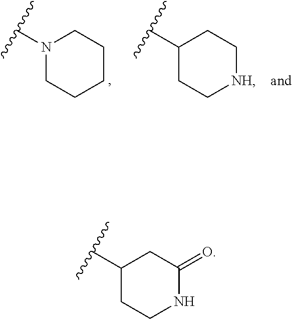 [SMILES: *N1CCCCC1 ; *C1CCNCC1 ; "and" ; *C1CCNC(=O)C1]

The term “aryl” as used herein by itself or as part of another group refers to an aromatic ring system having six to fourteen carbon atoms, i.e., C₆-C₁₄ aryl, C₉-C₁₀ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “heteroaryl” as used herein by itself or as part of another group refers to monocyclic and bicyclic aromatic ring systems having five to 14 fourteen ring members, i.e., a 5- to 14-membered heteroaryl, a 5- to 6-membered-heteroaryl, a 9- to 10-membered comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, O-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. Anon-limiting exemplary N-oxide is pyridyl N-oxide.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. In another embodiment, the alkyl is substituted with one optionally substituted cycloalkyl group. In another embodiment, the alkyl is substituted with two optionally substituted cycloalkyl groups. Non-limiting exemplary (cycloalkyl)alkyl groups include:

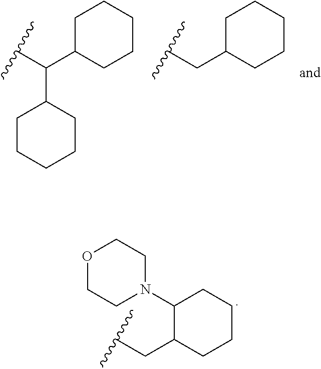

The term “carboxy” as used by itself or as part of another group refers to a radical of the formula —C(═O)OH.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom. Non-limiting exemplary (heterocyclo)alkyl groups include:

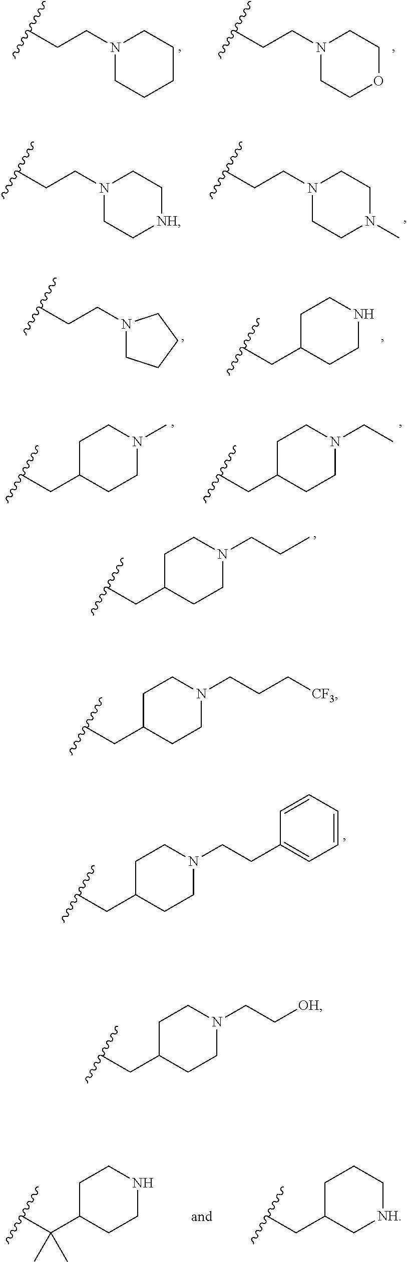

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl. Non-limiting exemplary (heteroaryl)alkyl groups include:

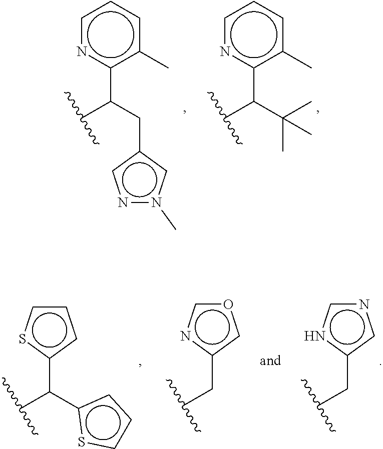

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a compound wherein substantially all of the atoms at a position within the Compound of the Disclosure are replaced by an atom having a different atomic mass or mass number. In another embodiment, provided is a compound wherein substantially all of the atoms at a position within the Compound of the Disclosure are replaced by deuterium atoms, e.g., all of the hydrogen atoms of a —CH₃ group are replaced by deuterium atoms to give a —CD₃ group. In another embodiment, provided is a compound wherein a portion of the atoms at a position within the Compound of the disclosure are replaced, i.e., the Compound of the Disclosure is enriched at a position with an atom having a different atomic mass or mass number. In another embodiment, provided is a compound wherein none of the atoms of the Compound of the Disclosure are replaced by an atom having a different atomic mass or mass number. Isotopically-labelled Compounds of the Disclosure can be prepared by methods known in the art.

Compounds of the Disclosure contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number±10%. Thus, “about 10” means 9 to 11.

### II. Compounds

In one aspect, the present disclosure provides a compound of Formula I:

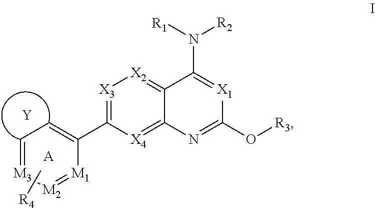

wherein:
X₁, X₂, X₃ and X₄ independently represent C or N, and when X₁, X₂, X₃ or X₄ represents C, it is optionally substituted with C₁-C₄ alkyl, halogen, hydroxyl or CN;
R₁ represents 5-membered or 6-membered heterocyclyl or heteroaryl group containing 1, 2 or 3 N atoms, and R₂ represents H or C₁-C₄ alkyl, wherein the heterocyclyl or heteroaryl is optionally substituted with one or more of C₁-C₄ alkyl, amino, halogen, or —C₁-C₄ alkyl-CN; or R₁ and R₂ taken together with the nitrogen atom to which they are attached form a N-containing heterocyclyl, wherein the heterocyclyl is optionally substituted with one or more of C₁-C₄ alkyl, amino, halogen, hydroxyl or —C₁-C₄ alkyl-CN;
R₃ is selected from the group consisting of (C₃-C₆ cycloalkyl)C₁-C₄ alkyl, (heterocyclo)C₁-C₄ alkyl, (aryl)C₁-C₄ alkyl, and (hetereoaryl)C₁-C₄ alkyl; wherein the cycloalkyl, heterocyclo, aryl and hetereoaryl are optionally substituted with one or more of halogen, C₁-C₄ alkyl, —N(C₁-C₄ alkyl)₂, heterocyclyl, C₁-C₄ alkoxy, (hydroxy)C₁-C₄ alkyl, (amino)C₁-C₄ alkyl, —C₁-C₄ alkyl-O—C(═O)—N(C₁-C₄ alkyl)₂, —C₁-C₄ alkyl-NH—C(═O)—N(C₁-C₄ alkyl)₂, —O—C(═O)-heterocyclyl, —C₁-C₄ alkyl-N(C₁-C₄ alkyl)₂, or —C₁-C₄ alkyl-heterocyclyl;
M₁, M₂ and M₃ independently represent C or N, and when M₁, M₂ or M₃ represents C, it is optionally substituted with R₄, wherein R₄ represents hydroxyl, halogen, C₁-C₄ alkyl or amino;
\textcircled{Y} represents a bicyclic ring system fused to ring A, wherein the bicyclic ring system is selected from the group consisting of a bicyclic heterocyclyl, a bicyclic aryl, a bicyclic hetereoaryl, or a monocyclic aryl, heteroaryl or carbocyclic fused with monocyclic carbocyclic or heterocyclic ring, wherein the bicyclic ring system is optionally substituted with one or more of halogen, C₁-C₄ alkyl, oxo, (hydroxyl)C₁-C₄ alkyl or hydroxyl;
or a pharmaceutically acceptable salt or solvate thereof.

In another aspect, the present disclosure provides a compound of Formula Ia:

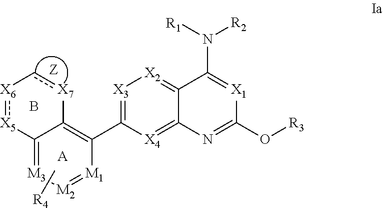

wherein:
X₅, X₆ and X₇ independently represent C or N;
each [symbol] in ring B independently represents a single or double bond,
\textcircled{Z} represents a 5-7 membered carbocyclic, 5-7 membered aryl or 5-7-membered heterocyclyl or hetereoaryl fused with ring B having 1-3 heteroatoms selected from the group consisting of N, S and O; wherein the carbocyclic, aryl, heterocyclyl or hetereoaryl is optionally substituted with one or more of halogen, C₁-C₄ alkyl, oxo, (hydroxyl)C₁-C₄ alkyl or hydroxyl;
and other groups are as defined above,
or a pharmaceutically acceptable salt or solvate thereof.

In another aspect, the present disclosure provides a compound of Formula Ib:

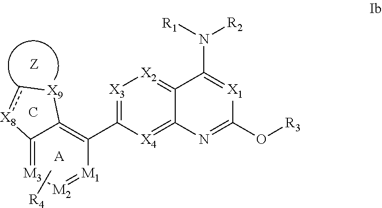

wherein:
X₈ and X₉ independently represent C or N;
\textcircled{Z} represents a fused 5-7 membered carbocyclic, 5-7 membered aryl or 5-7-membered heterocyclyl or hetereoaryl having 1-3 heteroatoms selected from the group consisting of N, S and O; wherein the carbocyclic, aryl, heterocyclyl or hetereoaryl is optionally substituted with one or more of halogen, C₁-C₄ alkyl, oxo, (hydroxyl)C₁-C₄ alkyl or hydroxyl;
[symbol] in ring C represents a single or double bond,
and other groups are as defined above,
or a pharmaceutically acceptable salt or solvate thereof.

In another aspect, the present disclosure provides a compound of Formula II:

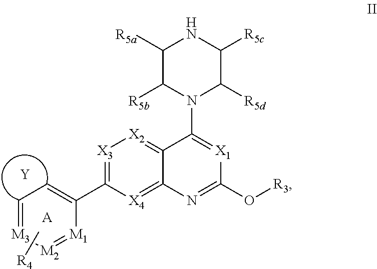

wherein:
X₁, X₂, X₃ and X₄ independently represent C or N, and when X₁, X₂, X₃ or X₄ represents C, it is optionally substituted with C₁-C₄ alkyl, halogen, hydroxyl or CN;
R³ is selected from the group consisting of (C₃-C₆ cycloalkyl)C₁-C₄ alkyl, (heterocyclo)C₁-C₄ alkyl, (aryl)C₁-C₄ alkyl, and (hetereoaryl)C₁-C₄ alkyl; wherein the cycloalkyl, heterocyclo, aryl and hetereoaryl are optionally substituted with one or more of halogen, C₁-C₄ alkyl, —N(C₁-C₄ alkyl)₂, heterocyclyl, C₁-C₄ alkoxy, (hydroxy)C₁-C₄ alkyl, (amino)C₁-C₄ alkyl, —C₁-C₄ alkyl-O—C(═O)—N(C₁-C₄ alkyl)₂, —C₁-C₄ alkyl-NH—C(═O)—N(C₁-C₄ alkyl)₂, —O—C(═O)-heterocyclyl, —C₁-C₄ alkyl-N(C₁-C₄ alkyl)₂, or —C₁-C₄ alkyl-heterocyclyl;
M₁, M₂ and M₃ independently represent C or N, and when M₁, M₂ or M₃ represents C, it is optionally substituted with R₄, wherein R₄ represents hydroxyl, halogen, C₁-C₄ alkyl or amino;
\textcircled{Y} represents a bicyclic ring system fused to ring A, wherein the bicyclic ring system is selected from the group consisting of a bicyclic heterocyclyl, a bicyclic aryl, a bicyclic hetereoaryl, or a monocyclic aryl, heteroaryl or carbocyclic fused with monocyclic carbocyclic or heterocyclic ring, wherein the bicyclic ring system is optionally substituted with one or more of halogen, C₁-C₄ alkyl, oxo, (hydroxyl)C₁-C₄ alkyl or hydroxyl;
R₅ₐ, R_{5b}, R_{5c} and R₅ₐ independently represent H, C₁-C₄ alkyl, —C₁-C₄ alkyl-CN, amino, halogen, hydroxyl or a bond; or any one of R₅ₐ and R_{5b} and any one of R_{5c} and R₅ₐ are linked together with a —(CH₂)ₙ— linker, wherein n is 1, 2 or 3;
or a pharmaceutically acceptable salt or solvate thereof.

In another aspect, the present disclosure provides a compound of Formula IIa:

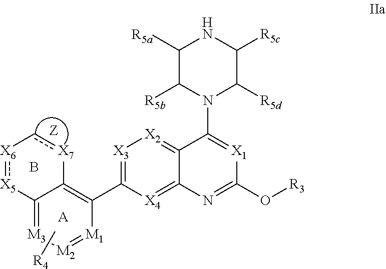

wherein:
X₅, X₆ and X₇ independently represent C or N;
each [symbol] in ring B independently represents a single or double bond,
\textcircled{Z} represents a 5-7 membered carbocyclic, 5-7 membered aryl or 5-7-membered heterocyclyl or hetereoaryl fused with ring B having 1-3 heteroatoms selected from the group consisting of N, S and O; wherein the carbocyclic, aryl, heterocyclyl or hetereoaryl is optionally substituted with one or more of halogen, C₁-C₄ alkyl, oxo, (hydroxyl)C₁-C₄ alkyl or hydroxyl;
other groups and parameters are as defined above,
or a pharmaceutically acceptable salt or solvate thereof.

In another aspect, the present disclosure provides a compound of Formula IIb:

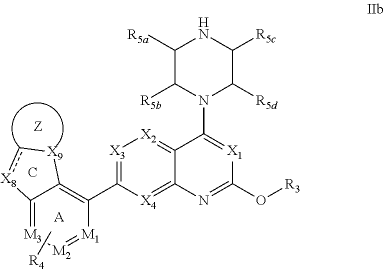

wherein:
X₈ and X₉ independently represent C or N;
\textcircled{Z} represents a 5-7 membered carbocyclic, 5-7 membered aryl or 5-7-membered heterocyclyl or hetereoaryl fused with ring C having 1-3 heteroatoms selected from the group consisting of N, S and O; wherein the carbocyclic, aryl, heterocyclyl or hetereoaryl is optionally substituted with one or more of halogen, C₁-C₄ alkyl, oxo, (hydroxyl)C₁-C₄ alkyl or hydroxyl;
[symbol] in ring C represents a single or double bond,
other groups and parameters are as defined above,
or a pharmaceutically acceptable salt or solvate thereof.

In some embodiments, for the above aspects of the disclosure, X₁ represents N, X₂ represents C, X₃ represents N, and X₄ represents C; or X₁ represents N, X₂ represents C, X₃ represents C, and X₄ represents C; or X₁ represents C, X₂ represents C, X₃ represents N, and X₄ represents C; or X₁ represents N, X₂ represents C, X₃ represents C, and X₄ represents N.

In some embodiments, for the above aspects of the disclosure, M₁, M₂ and M₃ represent C; and/or R₄ is a substitution group on M₂.

In some embodiments, for the above aspects of the disclosure, R₁ is selected from the following groups optionally substituted with one or more of C₁-C₄ alkyl, amino, halogen, or —C₁-C₄ alkyl-CN:

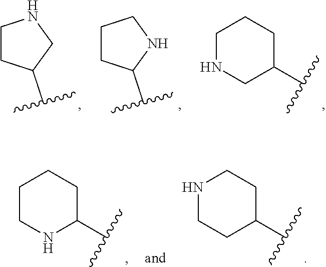 [SMILES: *C1CCNC1 ; *C1CCCN1 ; *C1CCCNC1 ; *C1CCCCN1 ; *C1CCNCC1]

In some embodiments, for the above aspects of the disclosure, R₂ is H.

In some embodiments, for the above aspects of the disclosure, R₁ and R₂ taken together with the nitrogen atom to which they are attached form a N-containing heterocyclyl as follows optionally substituted with one or more of C₁-C₄ alkyl, amino, halogen, hydroxyl, or —C₁-C₄ alkyl-CN:

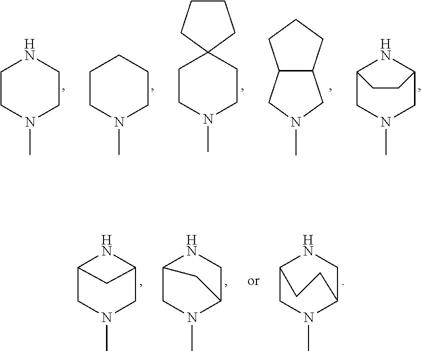

In some embodiments, for the above aspects of the disclosure, the C₃-C₆ cycloalkyl in R₃ moiety is selected from the group consisting of cyclopropyl, cyclobutyl, cyclopentyl or cyclohexyl.

In some embodiments, for the above aspects of the disclosure, the heterocyclo in R₃ moiety is selected from the group consisting of:

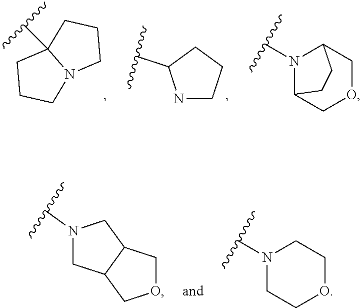

In some embodiments, for the above aspects of the disclosure, R₃ is selected from the group consisting of:

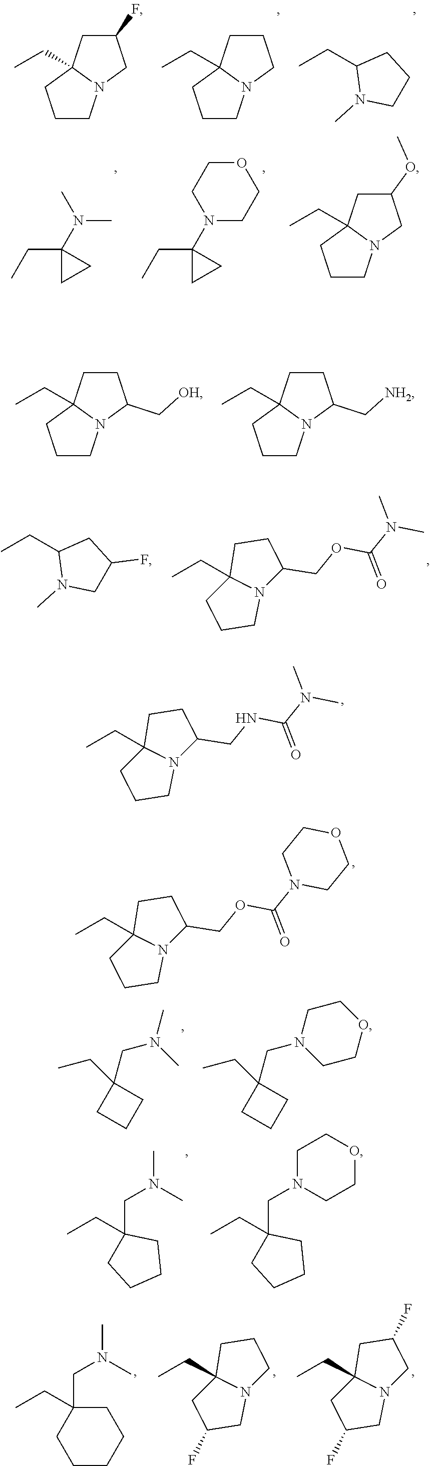

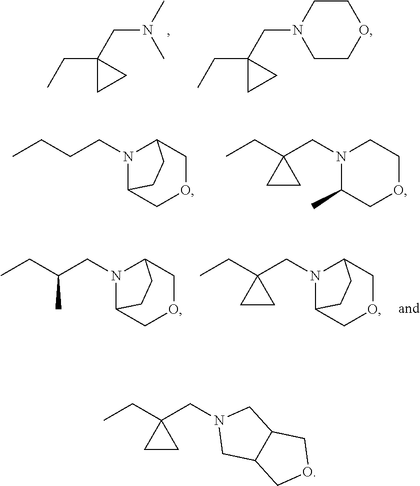

In some embodiments, for the above aspects of the disclosure, R⁴ is selected from the group consisting of: hydroxyl and amino.

In some embodiments, for the above aspects of the disclosure, the bicyclic ring system in \textcircled{Y} moiety is selected from the group consisting of:

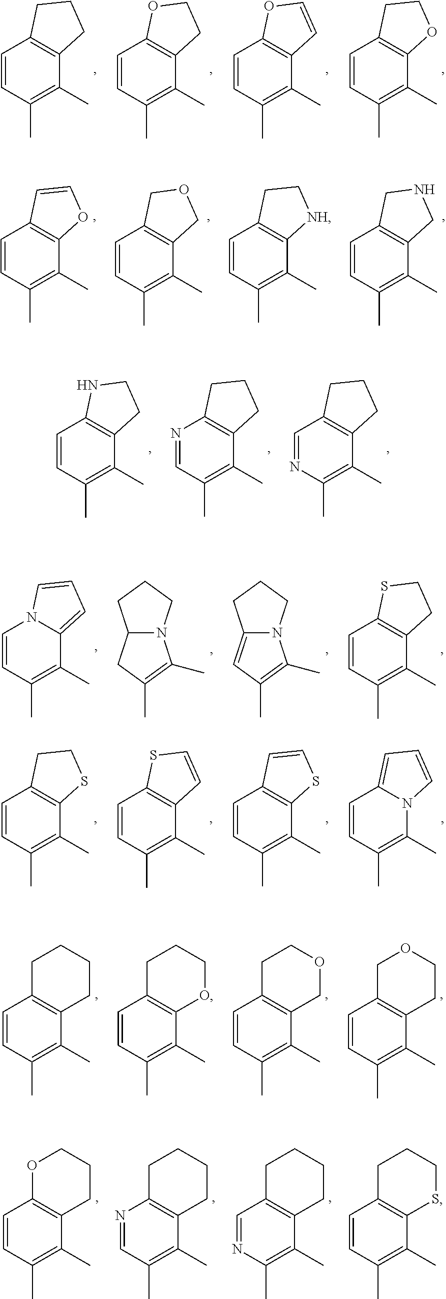

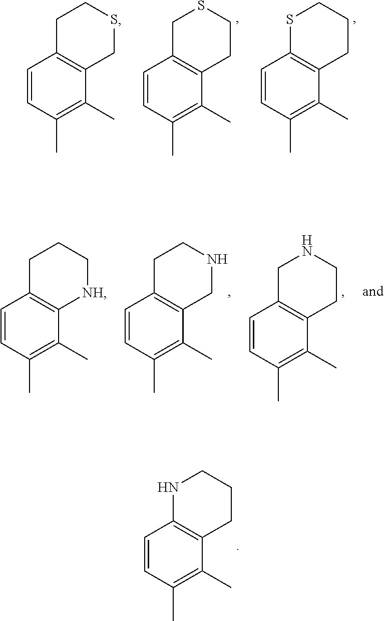

In some embodiments, for the above aspects of the disclosure, the optional substituent in \textcircled{Y} moiety is methyl or F.

In some embodiments, for the above aspects of the disclosure, \textcircled{Z} represents a fused 5 membered carbocyclic, 5 membered aryl or 5-membered heterocyclyl or hetereoaryl having 1-3 heteroatoms selected from the group consisting of N, S and O; wherein the carbocyclic, aryl, heterocyclyl or hetereoaryl is optionally substituted with one or more of halogen, C₁-C₄ alkyl, or hydroxyl.

In some embodiments, for the above aspects of the disclosure, \textcircled{Z} represents a fused 6 membered carbocyclic, 6 membered aryl or 6-membered heterocyclyl or hetereoaryl having 1-3 heteroatoms selected from the group consisting of N, S and O; wherein the carbocyclic, aryl, heterocyclyl or hetereoaryl is optionally substituted with one or more of halogen, C₁-C₄ alkyl, or hydroxyl.

In some embodiments, for the above aspects of the disclosure, each -̅-̅-̅ in ring B represents a double bond.

In some embodiments, for the above aspects of the disclosure, the compound is selected from the group consisting of

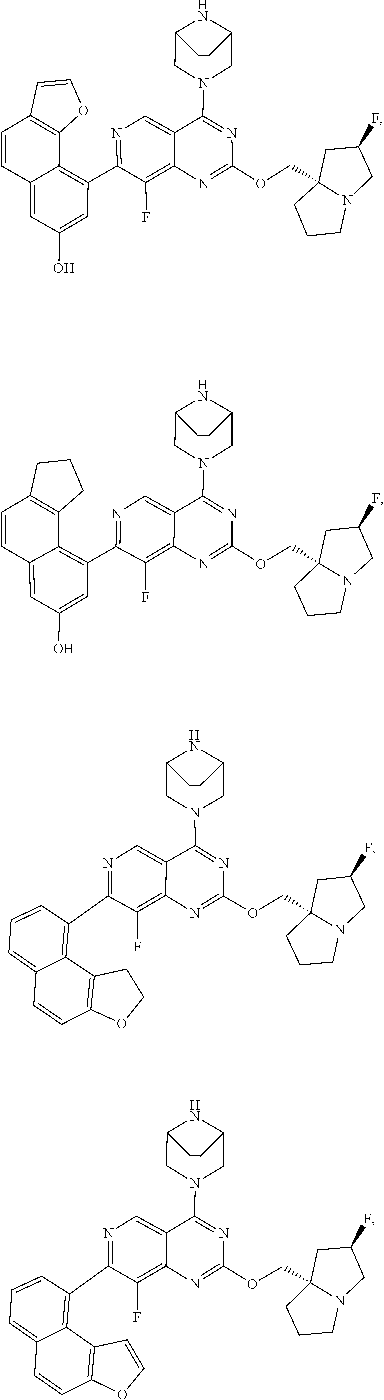 [SMILES: Oc1cc(-c2ncc3c(N4CC5CCC(C4)N5)nc(OC[C@@]45CCCN4C[C@H](F)C5)nc3c2F)c2c(ccc3ccoc32)c1 ; Oc1cc(-c2ncc3c(N4CC5CCC(C4)N5)nc(OC[C@@]45CCCN4C[C@H](F)C5)nc3c2F)c2c3c(ccc2c1)CCC3 ; Fc1c(-c2cccc3ccc4c(c23)CCO4)ncc2c(N3CC4CCC(C3)N4)nc(OC[C@@]34CCCN3C[C@H](F)C4)nc12 ; Fc1c(-c2cccc3ccc4occc4c23)ncc2c(N3CC4CCC(C3)N4)nc(OC[C@@]34CCCN3C[C@H](F)C4)nc12]

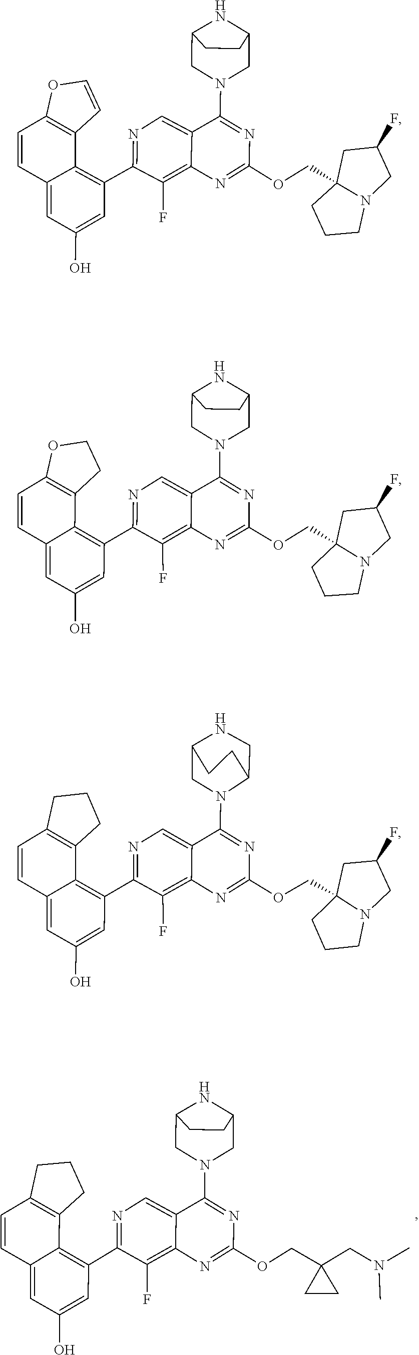 [SMILES: Oc1cc(-c2ncc3c(N4CC5CCC(C4)N5)nc(OC[C@@]45CCCN4C[C@H](F)C5)nc3c2F)c2c(ccc3occc32)c1 ; Oc1cc(-c2ncc3c(N4CC5CCC(C4)N5)nc(OC[C@@]45CCCN4C[C@H](F)C5)nc3c2F)c2c3c(ccc2c1)OCC3 ; Oc1cc(-c2ncc3c(N4CC5CCC4CN5)nc(OC[C@@]45CCCN4C[C@H](F)C5)nc3c2F)c2c3c(ccc2c1)CCC3 ; CN(C)CC1(COc2nc(N3CC4CCC(C3)N4)c3cnc(-c4cc(O)cc5ccc6c(c45)CCC6)c(F)c3n2)CC1]

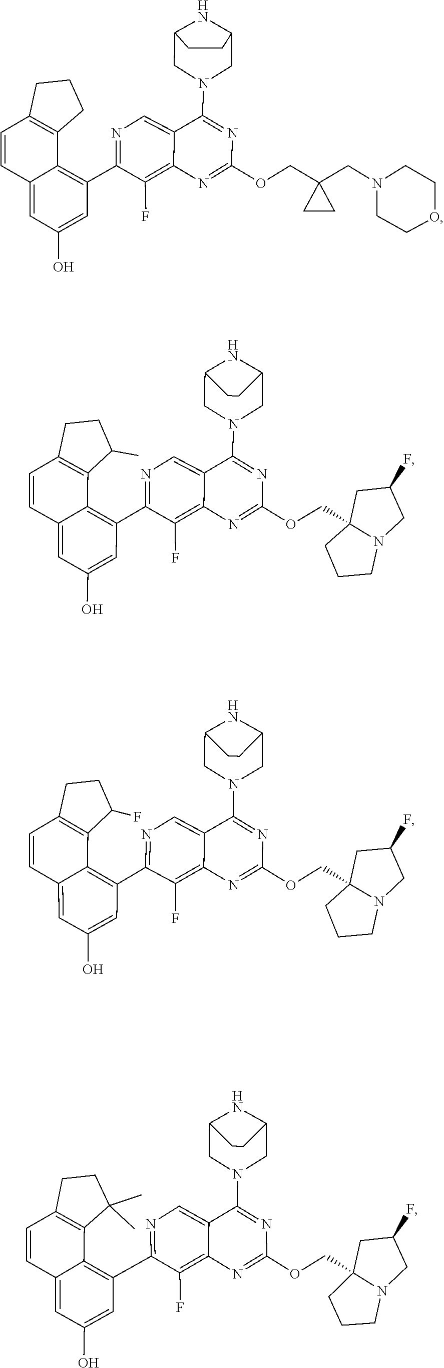 [SMILES: Oc1cc(-c2ncc3c(N4CC5CCC(C4)N5)nc(OCC4(CN5CCOCC5)CC4)nc3c2F)c2c3c(ccc2c1)CCC3 ; CC1CCc2ccc3cc(O)cc(-c4ncc5c(N6CC7CCC(C6)N7)nc(OC[C@@]67CCCN6C[C@H](F)C7)nc5c4F)c3c21 ; Oc1cc(-c2ncc3c(N4CC5CCC(C4)N5)nc(OC[C@@]45CCCN4C[C@H](F)C5)nc3c2F)c2c3c(ccc2c1)CCC3F ; CC1(C)CCc2ccc3cc(O)cc(-c4ncc5c(N6CC7CCC(C6)N7)nc(OC[C@@]67CCCN6C[C@H](F)C7)nc5c4F)c3c21]

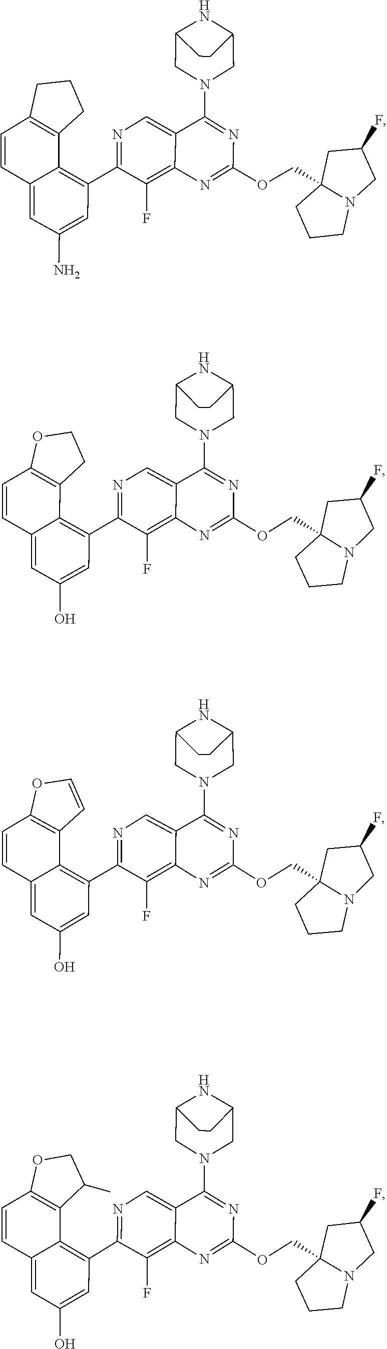 [SMILES: Nc1cc(-c2ncc3c(N4CC5CCC(C4)N5)nc(OC[C@@]45CCCN4C[C@H](F)C5)nc3c2F)c2c3c(ccc2c1)CCC3 ; Oc1cc(-c2ncc3c(N4CC5CCC(C4)N5)nc(OC[C@@]45CCCN4C[C@H](F)C5)nc3c2F)c2c3c(ccc2c1)OCC3 ; Oc1cc(-c2ncc3c(N4CC5CCC(C4)N5)nc(OC[C@@]45CCCN4C[C@H](F)C5)nc3c2F)c2c(ccc3occc32)c1 ; CC1COc2ccc3cc(O)cc(-c4ncc5c(N6CC7CCC(C6)N7)nc(OC[C@@]67CCCN6C[C@H](F)C7)nc5c4F)c3c21]

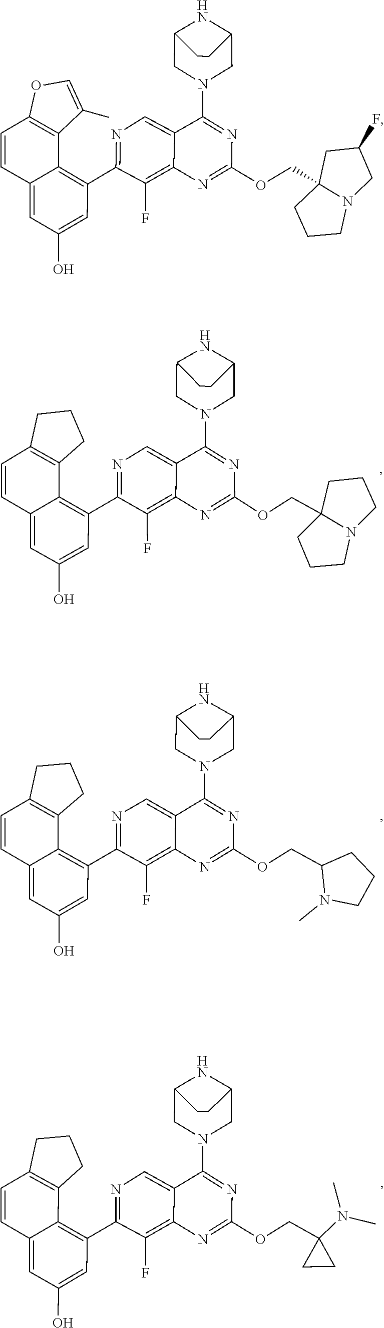 [SMILES: Cc1coc2ccc3cc(O)cc(-c4ncc5c(N6CC7CCC(C6)N7)nc(OC[C@@]67CCCN6C[C@H](F)C7)nc5c4F)c3c12 ; Oc1cc(-c2ncc3c(N4CC5CCC(C4)N5)nc(OCC45CCCN4CCC5)nc3c2F)c2c3c(ccc2c1)CCC3 ; CN1CCCC1COc1nc(N2CC3CCC(C2)N3)c2cnc(-c3cc(O)cc4ccc5c(c34)CCC5)c(F)c2n1 ; CN(C)C1(COc2nc(N3CC4CCC(C3)N4)c3cnc(-c4cc(O)cc5ccc6c(c45)CCC6)c(F)c3n2)CC1]

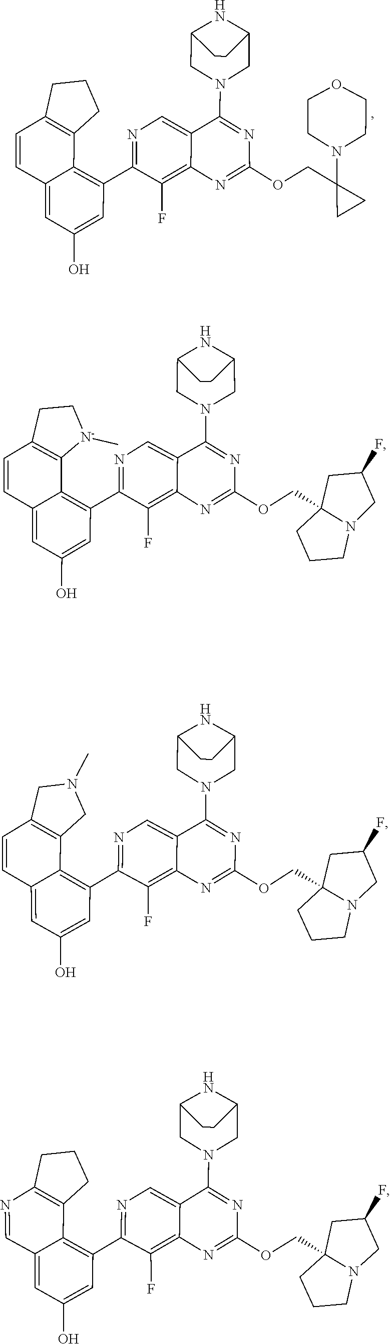

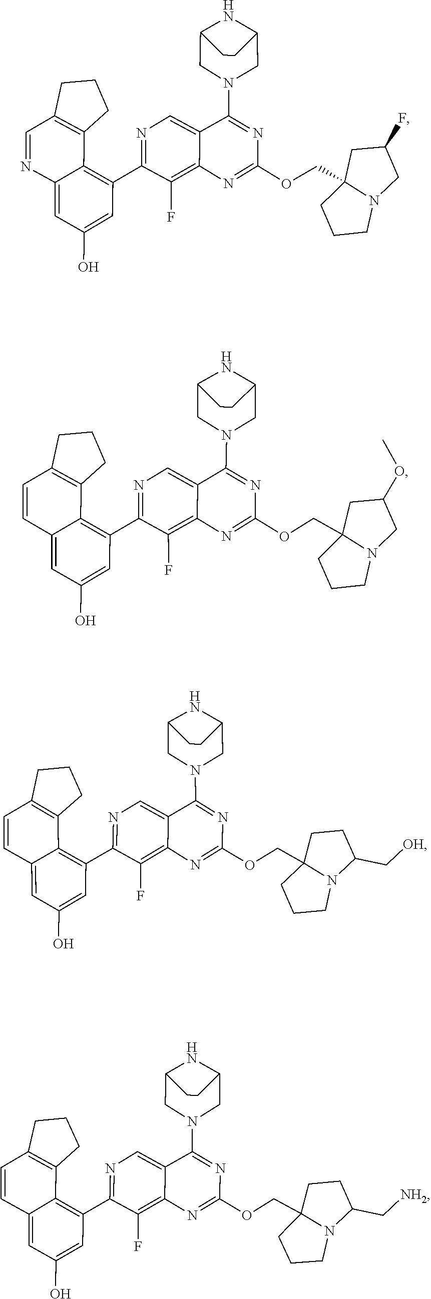 [SMILES: Oc1cc(-c2ncc3c(N4CC5CCC(C4)N5)nc(OC[C@@]45CCCN4C[C@H](F)C5)nc3c2F)c2c3c(cnc2c1)CCC3 ; COC1CN2CCCC2(COc2nc(N3CC4CCC(C3)N4)c3cnc(-c4cc(O)cc5ccc6c(c45)CCC6)c(F)c3n2)C1 ; OCC1CCC2(COc3nc(N4CC5CCC(C4)N5)c4cnc(-c5cc(O)cc6ccc7c(c56)CCC7)c(F)c4n3)CCCN12 ; NCC1CCC2(COc3nc(N4CC5CCC(C4)N5)c4cnc(-c5cc(O)cc6ccc7c(c56)CCC7)c(F)c4n3)CCCN12]

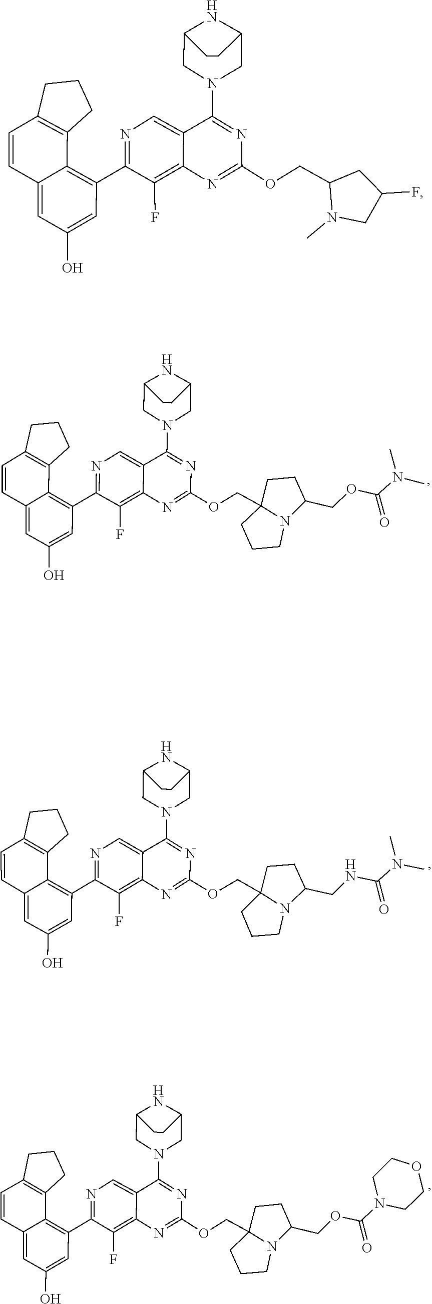 [SMILES: CN1CC(F)CC1COc1nc(N2CC3CCC(C2)N3)c2cnc(-c3cc(O)cc4ccc5c(c34)CCC5)c(F)c2n1 ; CN(C)C(=O)OCC1CCC2(COc3nc(N4CC5CCC(C4)N5)c4cnc(-c5cc(O)cc6ccc7c(c56)CCC7)c(F)c4n3)CCCN12 ; CN(C)C(=O)NCC1CCC2(COc3nc(N4CC5CCC(C4)N5)c4cnc(-c5cc(O)cc6ccc7c(c56)CCC7)c(F)c4n3)CCCN12 ; O=C(OCC1CCC2(COc3nc(N4CC5CCC(C4)N5)c4cnc(-c5cc(O)cc6ccc7c(c56)CCC7)c(F)c4n3)CCCN12)N1CCOCC1]

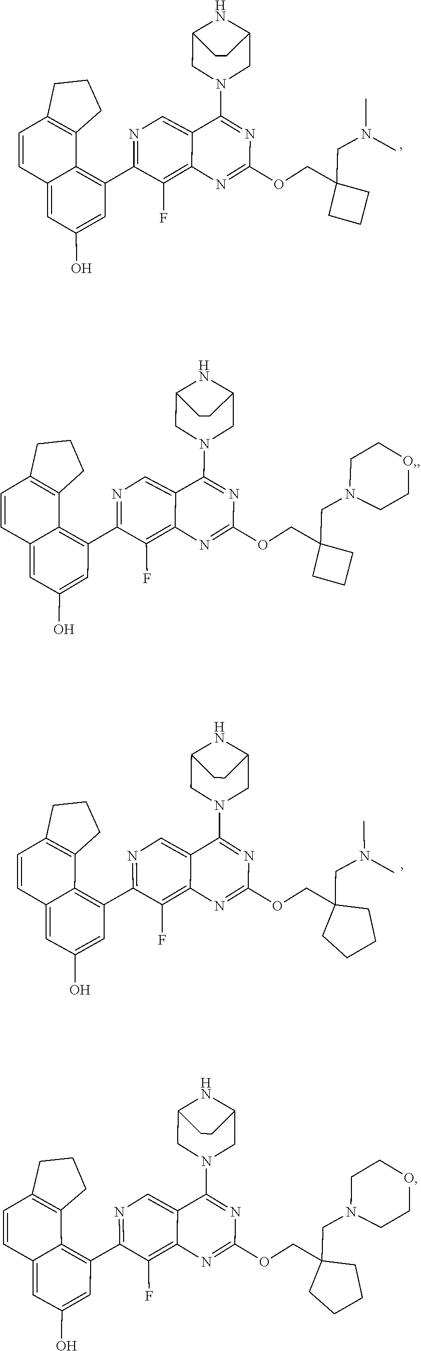 [SMILES: CN(C)CC1(COc2nc(N3CC4CCC(C3)N4)c3cnc(-c4cc(O)cc5ccc6c(c45)CCC6)c(F)c3n2)CCC1 ; Oc1cc(-c2ncc3c(N4CC5CCC(C4)N5)nc(OCC4(CN5CCOCC5)CCC4)nc3c2F)c2c3c(ccc2c1)CCC3 ; CN(C)CC1(COc2nc(N3CC4CCC(C3)N4)c3cnc(-c4cc(O)cc5ccc6c(c45)CCC6)c(F)c3n2)CCCC1 ; Oc1cc(-c2ncc3c(N4CC5CCC(C4)N5)nc(OCC4(CN5CCOCC5)CCCC4)nc3c2F)c2c3c(ccc2c1)CCC3]

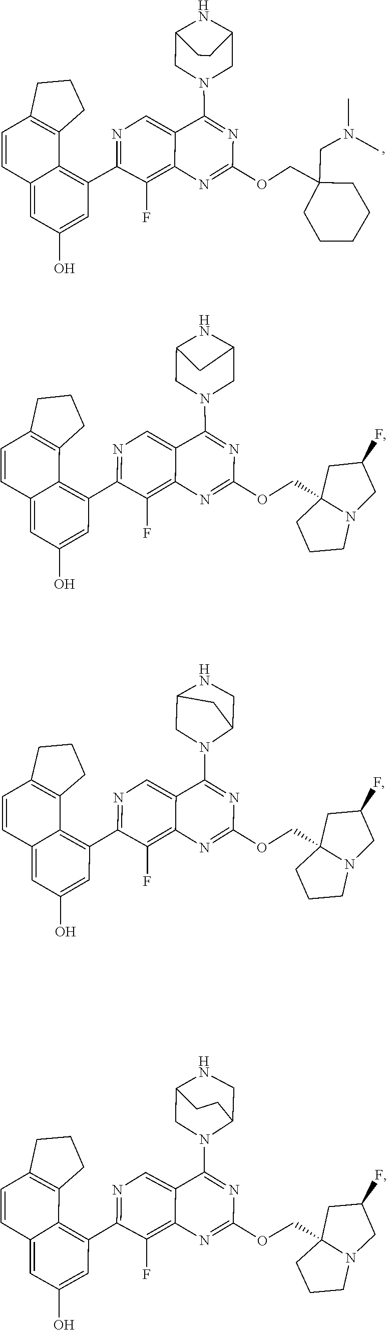 [SMILES: CN(C)CC1(COc2nc(N3CC4CCC(C3)N4)c3cnc(-c4cc(O)cc5ccc6c(c45)CCC6)c(F)c3n2)CCCCC1 ; Oc1cc(-c2ncc3c(N4CC5CC(C4)N5)nc(OC[C@@]45CCCN4C[C@H](F)C5)nc3c2F)c2c3c(ccc2c1)CCC3 ; Oc1cc(-c2ncc3c(N4CC5CC4CN5)nc(OC[C@@]45CCCN4C[C@H](F)C5)nc3c2F)c2c3c(ccc2c1)CCC3 ; Oc1cc(-c2ncc3c(N4CC5CCC4CN5)nc(OC[C@@]45CCCN4C[C@H](F)C5)nc3c2F)c2c3c(ccc2c1)CCC3]

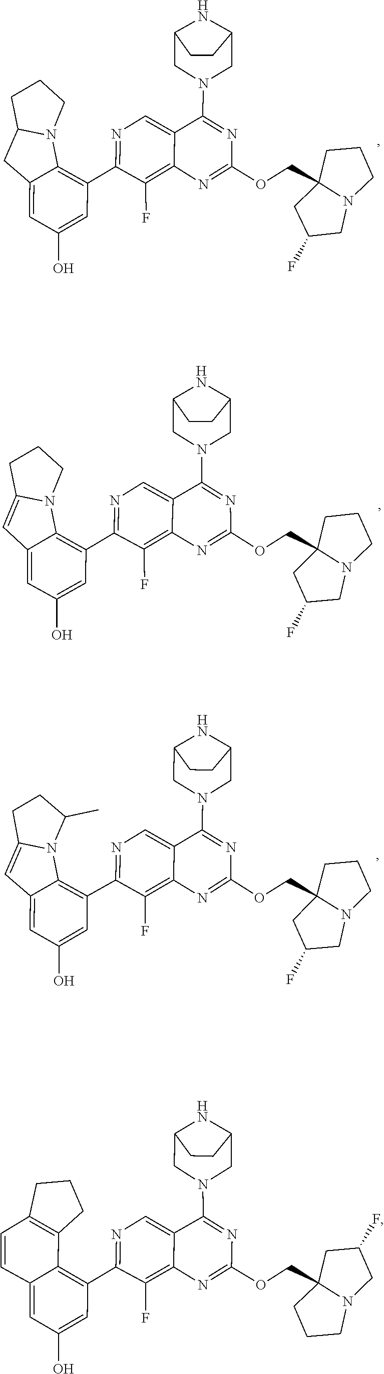 [SMILES: Oc1cc2c(c(-c3ncc4c(N5CC6CCC(C5)N6)nc(OC[C@@]56CCCN5C[C@H](F)C6)nc4c3F)c1)N1CCCC1C2 ; Oc1cc(-c2ncc3c(N4CC5CCC(C4)N5)nc(OC[C@@]45CCCN4C[C@H](F)C5)nc3c2F)c2c(c1)cc1n2CCC1 ; CC1CCc2cc3cc(O)cc(-c4ncc5c(N6CC7CCC(C6)N7)nc(OC[C@@]67CCCN6C[C@H](F)C7)nc5c4F)c3n21 ; Oc1cc(-c2ncc3c(N4CC5CCC(C4)N5)nc(OC[C@]45CCCN4C[C@@H](F)C5)nc3c2F)c2c3c(ccc2c1)CCC3]

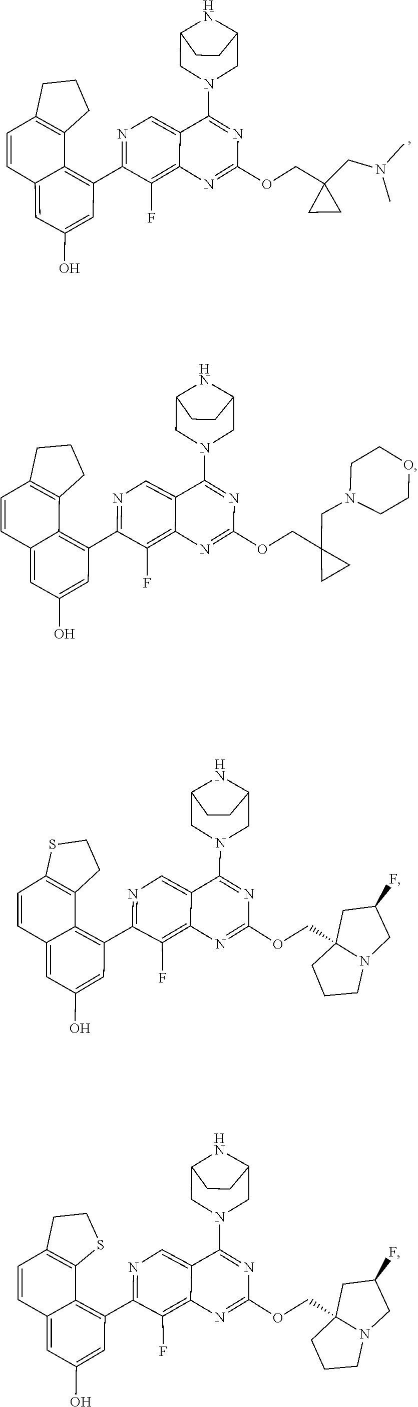 [SMILES: CN(C)CC1(COc2nc(N3CC4CCC(C3)N4)c3cnc(-c4cc(O)cc5ccc6c(c45)CCC6)c(F)c3n2)CC1 ; Oc1cc(-c2ncc3c(N4CC5CCC(C4)N5)nc(OCC4(CN5CCOCC5)CC4)nc3c2F)c2c3c(ccc2c1)CCC3 ; Oc1cc(-c2ncc3c(N4CC5CCC(C4)N5)nc(OC[C@@]45CCCN4C[C@H](F)C5)nc3c2F)c2c3c(ccc2c1)SCC3 ; Oc1cc(-c2ncc3c(N4CC5CCC(C4)N5)nc(OC[C@@]45CCCN4C[C@H](F)C5)nc3c2F)c2c3c(ccc2c1)CCS3]

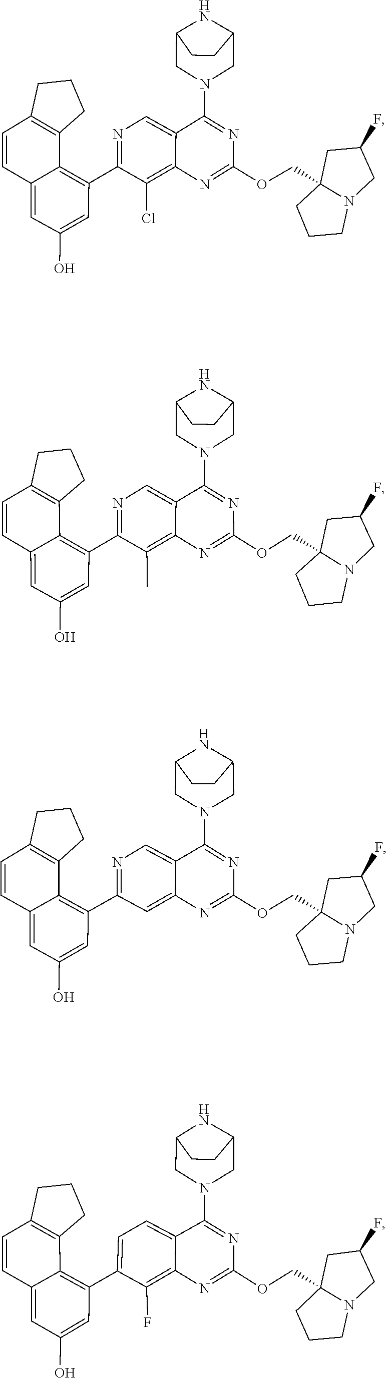 [SMILES: Oc1cc(-c2ncc3c(N4CC5CCC(C4)N5)nc(OC[C@@]45CCCN4C[C@H](F)C5)nc3c2Cl)c2c3c(ccc2c1)CCC3 ; Cc1c(-c2cc(O)cc3ccc4c(c23)CCC4)ncc2c(N3CC4CCC(C3)N4)nc(OC[C@@]34CCCN3C[C@H](F)C4)nc12 ; Oc1cc(-c2cc3nc(OC[C@@]45CCCN4C[C@H](F)C5)nc(N4CC5CCC(C4)N5)c3cn2)c2c3c(ccc2c1)CCC3 ; Oc1cc(-c2ccc3c(N4CC5CCC(C4)N5)nc(OC[C@@]45CCCN4C[C@H](F)C5)nc3c2F)c2c3c(ccc2c1)CCC3]

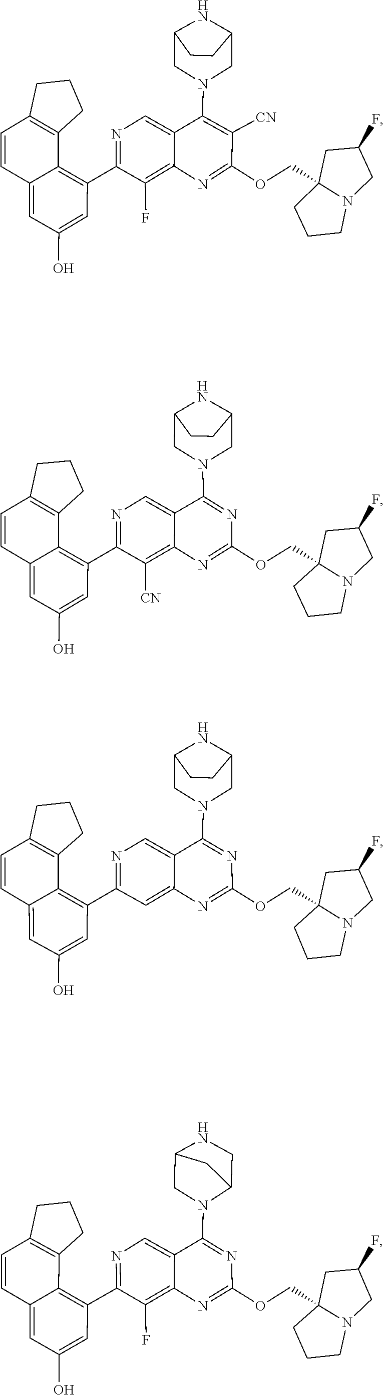 [SMILES: N#Cc1c(OC[C@@]23CCCN2C[C@H](F)C3)nc2c(F)c(-c3cc(O)cc4ccc5c(c34)CCC5)ncc2c1N1CC2CCC(C1)N2 ; N#Cc1c(-c2cc(O)cc3ccc4c(c23)CCC4)ncc2c(N3CC4CCC(C3)N4)nc(OC[C@@]34CCCN3C[C@H](F)C4)nc12 ; Oc1cc(-c2cc3nc(OC[C@@]45CCCN4C[C@H](F)C5)nc(N4CC5CCC(C4)N5)c3cn2)c2c3c(ccc2c1)CCC3 ; Oc1cc(-c2ncc3c(N4CC5CC4CN5)nc(OC[C@@]45CCCN4C[C@H](F)C5)nc3c2F)c2c3c(ccc2c1)CCC3]

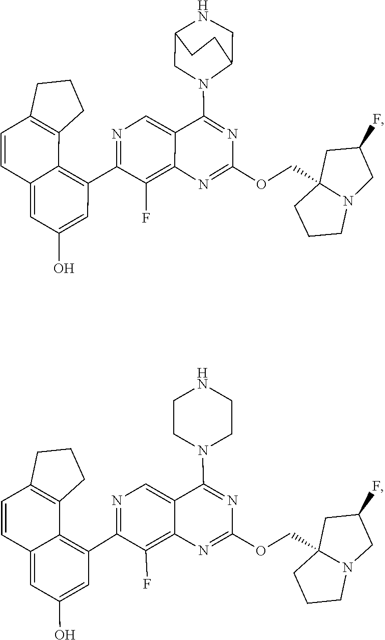 [SMILES: Oc1cc(-c2ncc3c(N4CC5CCC4CN5)nc(OC[C@@]45CCCN4C[C@H](F)C5)nc3c2F)c2c3c(ccc2c1)CCC3 ; Oc1cc(-c2ncc3c(N4CCNCC4)nc(OC[C@@]45CCCN4C[C@H](F)C5)nc3c2F)c2c3c(ccc2c1)CCC3]

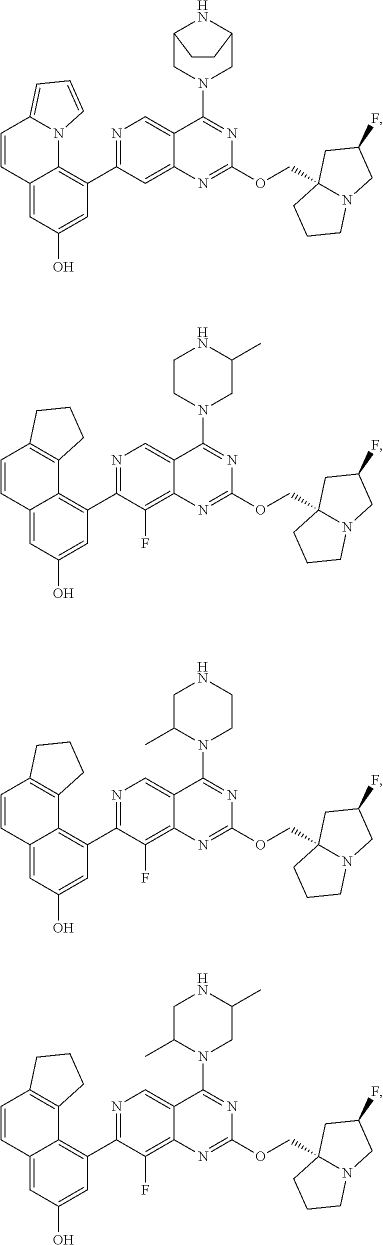 [SMILES: Oc1cc(-c2cc3nc(OC[C@@]45CCCN4C[C@H](F)C5)nc(N4CC5CCC(C4)N5)c3cn2)c2c(ccc3cccn32)c1 ; CC1CN(c2nc(OC[C@@]34CCCN3C[C@H](F)C4)nc3c(F)c(-c4cc(O)cc5ccc6c(c45)CCC6)ncc23)CCN1 ; CC1CNCCN1c1nc(OC[C@@]23CCCN2C[C@H](F)C3)nc2c(F)c(-c3cc(O)cc4ccc5c(c34)CCC5)ncc12 ; CC1CN(c2nc(OC[C@@]34CCCN3C[C@H](F)C4)nc3c(F)c(-c4cc(O)cc5ccc6c(c45)CCC6)ncc23)C(C)CN1]

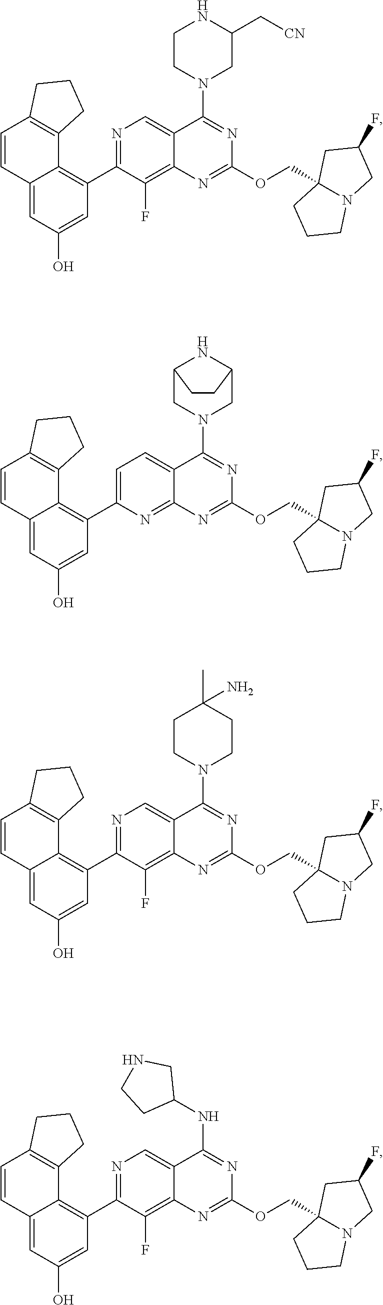 [SMILES: N#CCC1CN(c2nc(OC[C@@]34CCCN3C[C@H](F)C4)nc3c(F)c(-c4cc(O)cc5ccc6c(c45)CCC6)ncc23)CCN1 ; Oc1cc(-c2ccc3c(N4CC5CCC(C4)N5)nc(OC[C@@]45CCCN4C[C@H](F)C5)nc3n2)c2c3c(ccc2c1)CCC3 ; CC1(N)CCN(c2nc(OC[C@@]34CCCN3C[C@H](F)C4)nc3c(F)c(-c4cc(O)cc5ccc6c(c45)CCC6)ncc23)CC1 ; Oc1cc(-c2ncc3c(NC4CCNC4)nc(OC[C@@]45CCCN4C[C@H](F)C5)nc3c2F)c2c3c(ccc2c1)CCC3]

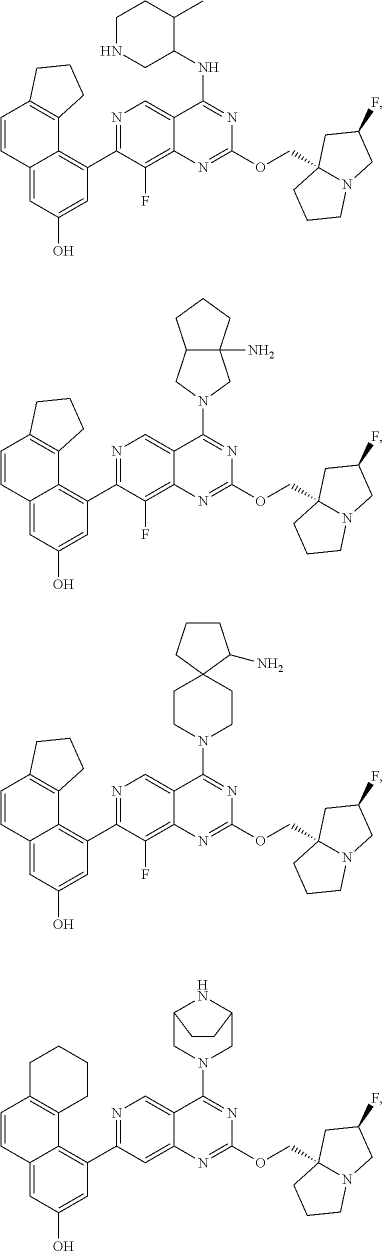 [SMILES: CC1CCNCC1Nc1nc(OC[C@@]23CCCN2C[C@H](F)C3)nc2c(F)c(-c3cc(O)cc4ccc5c(c34)CCC5)ncc12 ; NC12CCCC1CN(c1nc(OC[C@@]34CCCN3C[C@H](F)C4)nc3c(F)c(-c4cc(O)cc5ccc6c(c45)CCC6)ncc13)C2 ; NC1CCCC12CCN(c1nc(OC[C@@]34CCCN3C[C@H](F)C4)nc3c(F)c(-c4cc(O)cc5ccc6c(c45)CCC6)ncc13)CC2 ; Oc1cc(-c2cc3nc(OC[C@@]45CCCN4C[C@H](F)C5)nc(N4CC5CCC(C4)N5)c3cn2)c2c3c(ccc2c1)CCCC3]

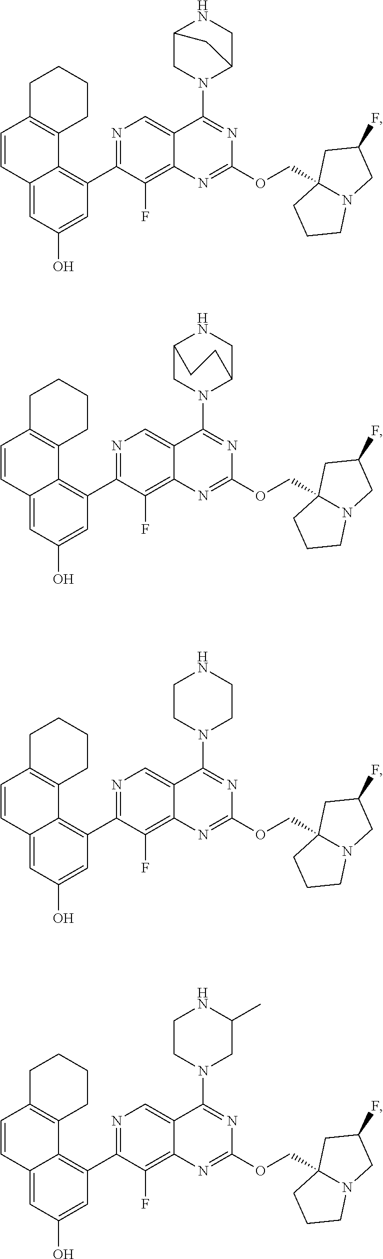 [SMILES: Oc1cc(-c2ncc3c(N4CC5CC4CN5)nc(OC[C@@]45CCCN4C[C@H](F)C5)nc3c2F)c2c3c(ccc2c1)CCCC3 ; Oc1cc(-c2ncc3c(N4CC5CCC4CN5)nc(OC[C@@]45CCCN4C[C@H](F)C5)nc3c2F)c2c3c(ccc2c1)CCCC3 ; Oc1cc(-c2ncc3c(N4CCNCC4)nc(OC[C@@]45CCCN4C[C@H](F)C5)nc3c2F)c2c3c(ccc2c1)CCCC3 ; CC1CN(c2nc(OC[C@@]34CCCN3C[C@H](F)C4)nc3c(F)c(-c4cc(O)cc5ccc6c(c45)CCCC6)ncc23)CCN1]

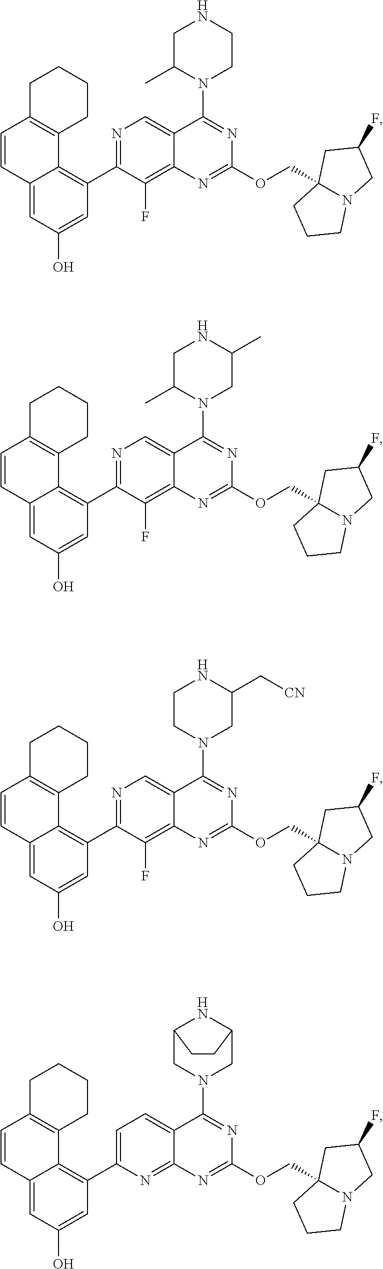 [SMILES: CC1CNCCN1c1nc(OC[C@@]23CCCN2C[C@H](F)C3)nc2c(F)c(-c3cc(O)cc4ccc5c(c34)CCCC5)ncc12 ; CC1CN(c2nc(OC[C@@]34CCCN3C[C@H](F)C4)nc3c(F)c(-c4cc(O)cc5ccc6c(c45)CCCC6)ncc23)C(C)CN1 ; N#CCC1CN(c2nc(OC[C@@]34CCCN3C[C@H](F)C4)nc3c(F)c(-c4cc(O)cc5ccc6c(c45)CCCC6)ncc23)CCN1 ; Oc1cc(-c2ccc3c(N4CC5CCC(C4)N5)nc(OC[C@@]45CCCN4C[C@H](F)C5)nc3n2)c2c3c(ccc2c1)CCCC3]

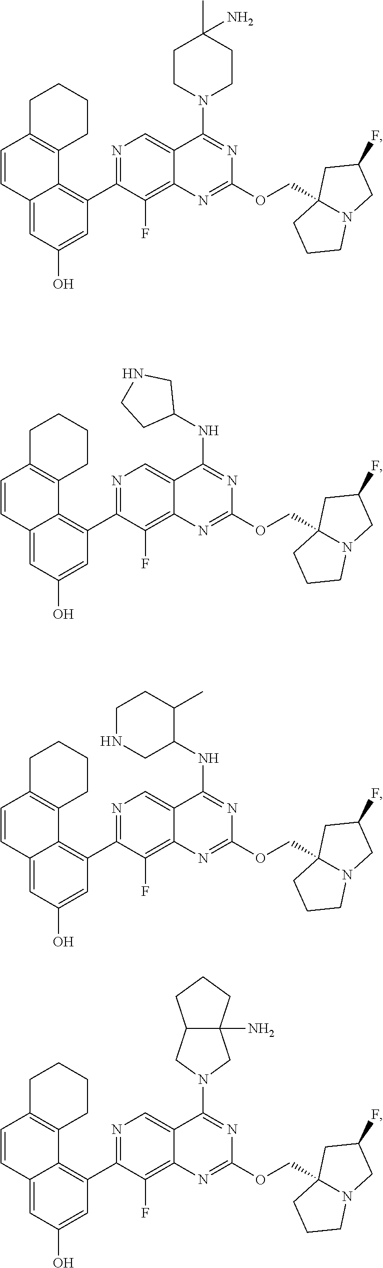 [SMILES: CC1(N)CCN(c2nc(OC[C@@]34CCCN3C[C@H](F)C4)nc3c(F)c(-c4cc(O)cc5ccc6c(c45)CCCC6)ncc23)CC1 ; Oc1cc(-c2ncc3c(NC4CCNC4)nc(OC[C@@]45CCCN4C[C@H](F)C5)nc3c2F)c2c3c(ccc2c1)CCCC3 ; CC1CCNCC1Nc1nc(OC[C@@]23CCCN2C[C@H](F)C3)nc2c(F)c(-c3cc(O)cc4ccc5c(c34)CCCC5)ncc12 ; NC12CCCC1CN(c1nc(OC[C@@]34CCCN3C[C@H](F)C4)nc3c(F)c(-c4cc(O)cc5ccc6c(c45)CCCC6)ncc13)C2]

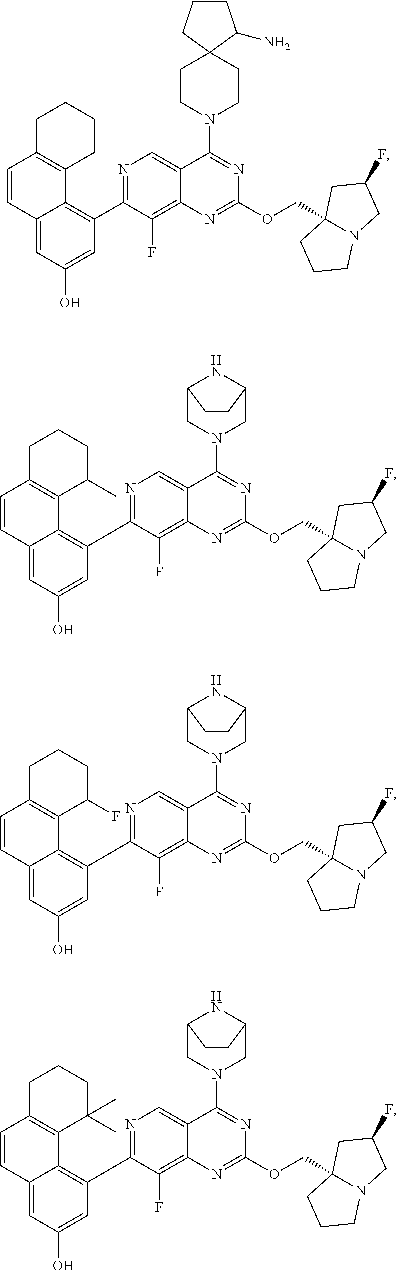 [SMILES: NC1CCCC12CCN(c1nc(OC[C@@]34CCCN3C[C@H](F)C4)nc3c(F)c(-c4cc(O)cc5ccc6c(c45)CCCC6)ncc13)CC2 ; CC1CCCc2ccc3cc(O)cc(-c4ncc5c(N6CC7CCC(C6)N7)nc(OC[C@@]67CCCN6C[C@H](F)C7)nc5c4F)c3c21 ; Oc1cc(-c2ncc3c(N4CC5CCC(C4)N5)nc(OC[C@@]45CCCN4C[C@H](F)C5)nc3c2F)c2c3c(ccc2c1)CCCC3F ; CC1(C)CCCc2ccc3cc(O)cc(-c4ncc5c(N6CC7CCC(C6)N7)nc(OC[C@@]67CCCN6C[C@H](F)C7)nc5c4F)c3c21]

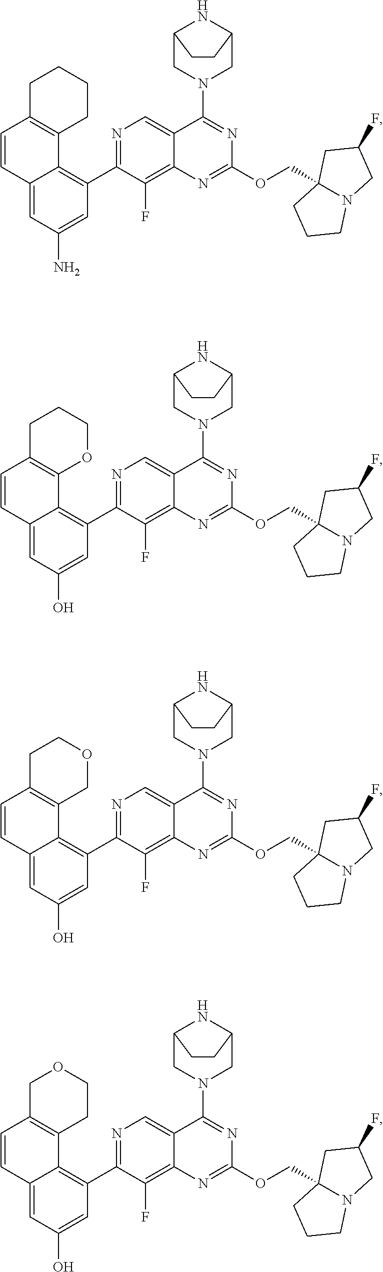 [SMILES: Nc1cc(-c2ncc3c(N4CC5CCC(C4)N5)nc(OC[C@@]45CCCN4C[C@H](F)C5)nc3c2F)c2c3c(ccc2c1)CCCC3 ; Oc1cc(-c2ncc3c(N4CC5CCC(C4)N5)nc(OC[C@@]45CCCN4C[C@H](F)C5)nc3c2F)c2c3c(ccc2c1)CCCO3 ; Oc1cc(-c2ncc3c(N4CC5CCC(C4)N5)nc(OC[C@@]45CCCN4C[C@H](F)C5)nc3c2F)c2c3c(ccc2c1)CCOC3 ; Oc1cc(-c2ncc3c(N4CC5CCC(C4)N5)nc(OC[C@@]45CCCN4C[C@H](F)C5)nc3c2F)c2c3c(ccc2c1)COCC3]

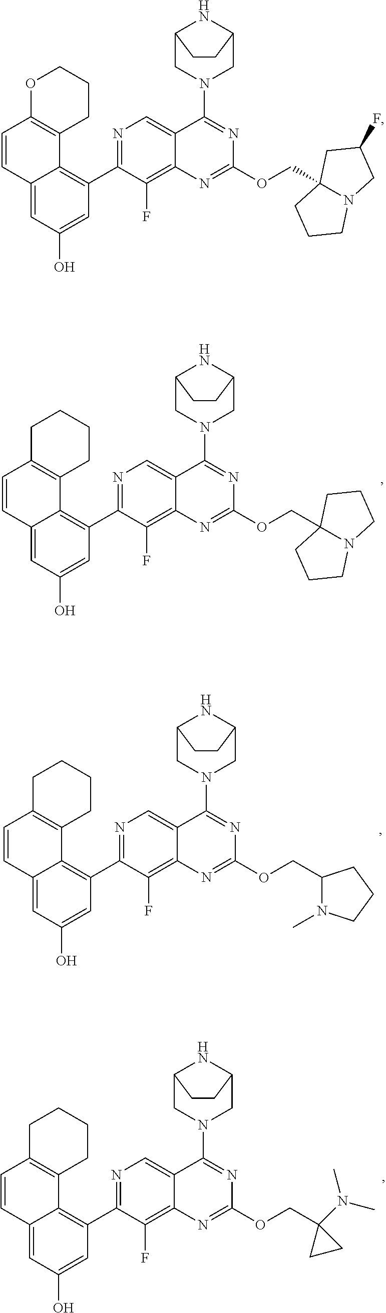 [SMILES: Oc1cc(-c2ncc3c(N4CC5CCC(C4)N5)nc(OC[C@@]45CCCN4C[C@H](F)C5)nc3c2F)c2c3c(ccc2c1)OCCC3 ; Oc1cc(-c2ncc3c(N4CC5CCC(C4)N5)nc(OCC45CCCN4CCC5)nc3c2F)c2c3c(ccc2c1)CCCC3 ; CN1CCCC1COc1nc(N2CC3CCC(C2)N3)c2cnc(-c3cc(O)cc4ccc5c(c34)CCCC5)c(F)c2n1 ; CN(C)C1(COc2nc(N3CC4CCC(C3)N4)c3cnc(-c4cc(O)cc5ccc6c(c45)CCCC6)c(F)c3n2)CC1]

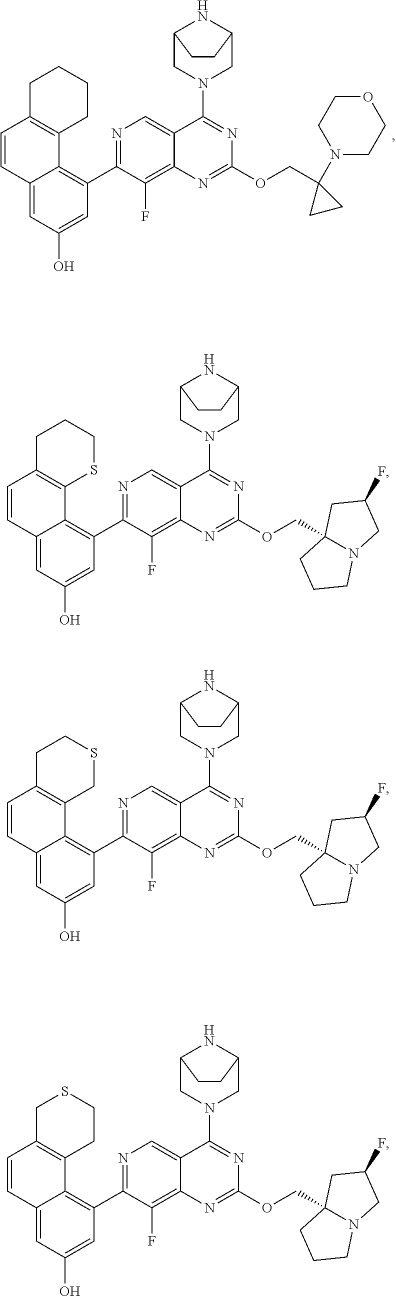 [SMILES: Oc1cc(-c2ncc3c(N4CC5CCC(C4)N5)nc(OCC4(N5CCOCC5)CC4)nc3c2F)c2c3c(ccc2c1)CCCC3 ; Oc1cc(-c2ncc3c(N4CC5CCC(C4)N5)nc(OC[C@@]45CCCN4C[C@H](F)C5)nc3c2F)c2c3c(ccc2c1)CCCS3 ; Oc1cc(-c2ncc3c(N4CC5CCC(C4)N5)nc(OC[C@@]45CCCN4C[C@H](F)C5)nc3c2F)c2c3c(ccc2c1)CCSC3 ; Oc1cc(-c2ncc3c(N4CC5CCC(C4)N5)nc(OC[C@@]45CCCN4C[C@H](F)C5)nc3c2F)c2c3c(ccc2c1)CSCC3]

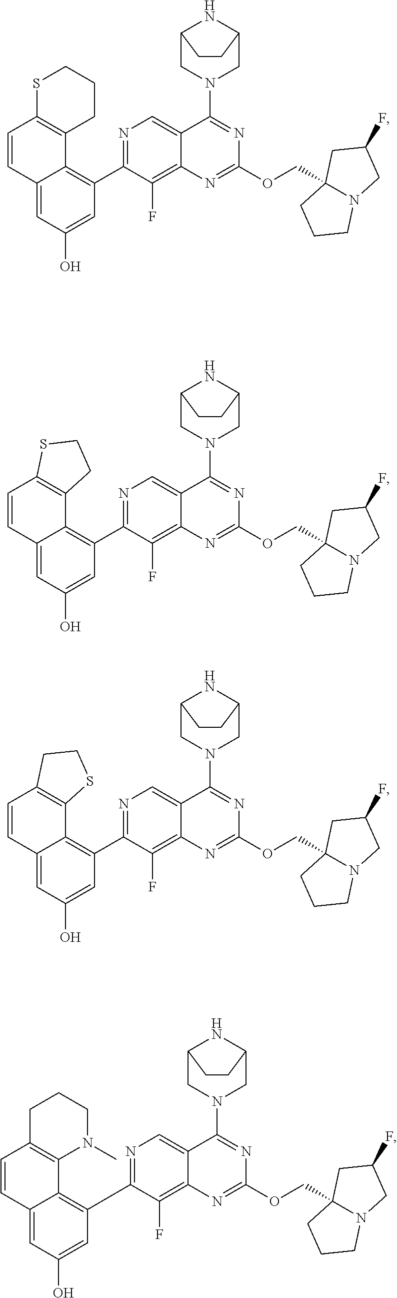 [SMILES: Oc1cc(-c2ncc3c(N4CC5CCC(C4)N5)nc(OC[C@@]45CCCN4C[C@H](F)C5)nc3c2F)c2c3c(ccc2c1)SCCC3 ; Oc1cc(-c2ncc3c(N4CC5CCC(C4)N5)nc(OC[C@@]45CCCN4C[C@H](F)C5)nc3c2F)c2c3c(ccc2c1)SCC3 ; Oc1cc(-c2ncc3c(N4CC5CCC(C4)N5)nc(OC[C@@]45CCCN4C[C@H](F)C5)nc3c2F)c2c3c(ccc2c1)CCS3 ; CN1CCCc2ccc3cc(O)cc(-c4ncc5c(N6CC7CCC(C6)N7)nc(OC[C@@]67CCCN6C[C@H](F)C7)nc5c4F)c3c21]

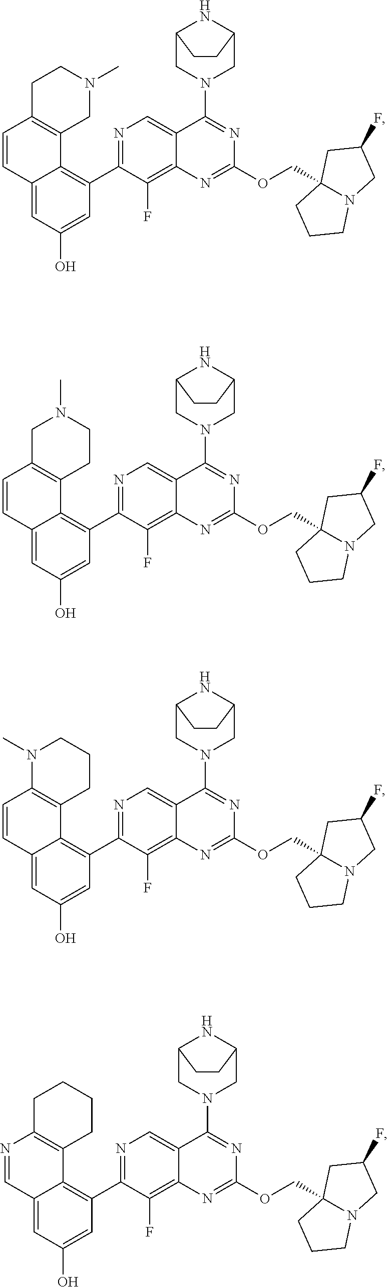 [SMILES: CN1CCc2ccc3cc(O)cc(-c4ncc5c(N6CC7CCC(C6)N7)nc(OC[C@@]67CCCN6C[C@H](F)C7)nc5c4F)c3c2C1 ; CN1CCc2c(ccc3cc(O)cc(-c4ncc5c(N6CC7CCC(C6)N7)nc(OC[C@@]67CCCN6C[C@H](F)C7)nc5c4F)c23)C1 ; CN1CCCc2c1ccc1cc(O)cc(-c3ncc4c(N5CC6CCC(C5)N6)nc(OC[C@@]56CCCN5C[C@H](F)C6)nc4c3F)c21 ; Oc1cc(-c2ncc3c(N4CC5CCC(C4)N5)nc(OC[C@@]45CCCN4C[C@H](F)C5)nc3c2F)c2c3c(ncc2c1)CCCC3]

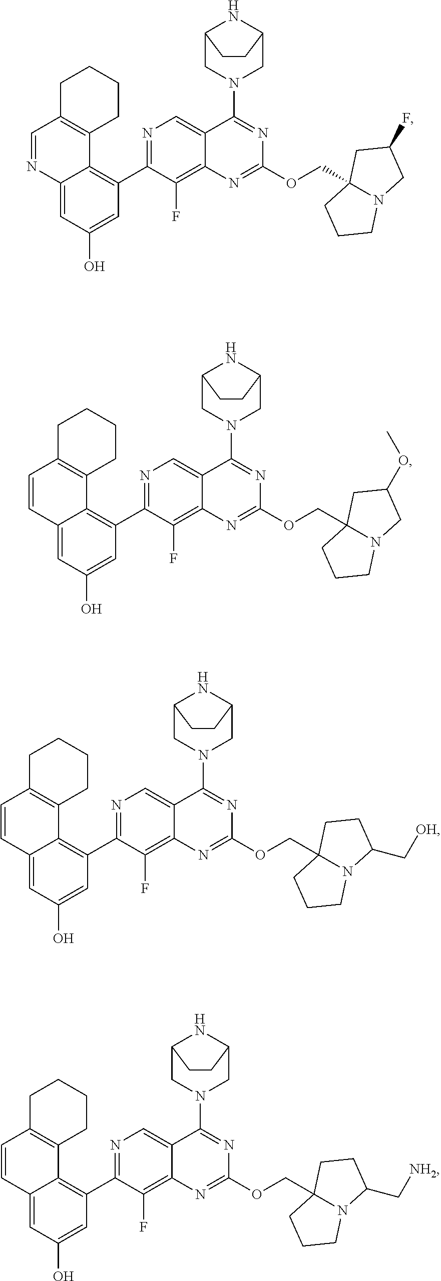 [SMILES: Oc1cc(-c2ncc3c(N4CC5CCC(C4)N5)nc(OC[C@@]45CCCN4C[C@H](F)C5)nc3c2F)c2c3c(cnc2c1)CCCC3 ; COC1CN2CCCC2(COc2nc(N3CC4CCC(C3)N4)c3cnc(-c4cc(O)cc5ccc6c(c45)CCCC6)c(F)c3n2)C1 ; OCC1CCC2(COc3nc(N4CC5CCC(C4)N5)c4cnc(-c5cc(O)cc6ccc7c(c56)CCCC7)c(F)c4n3)CCCN12 ; NCC1CCC2(COc3nc(N4CC5CCC(C4)N5)c4cnc(-c5cc(O)cc6ccc7c(c56)CCCC7)c(F)c4n3)CCCN12]

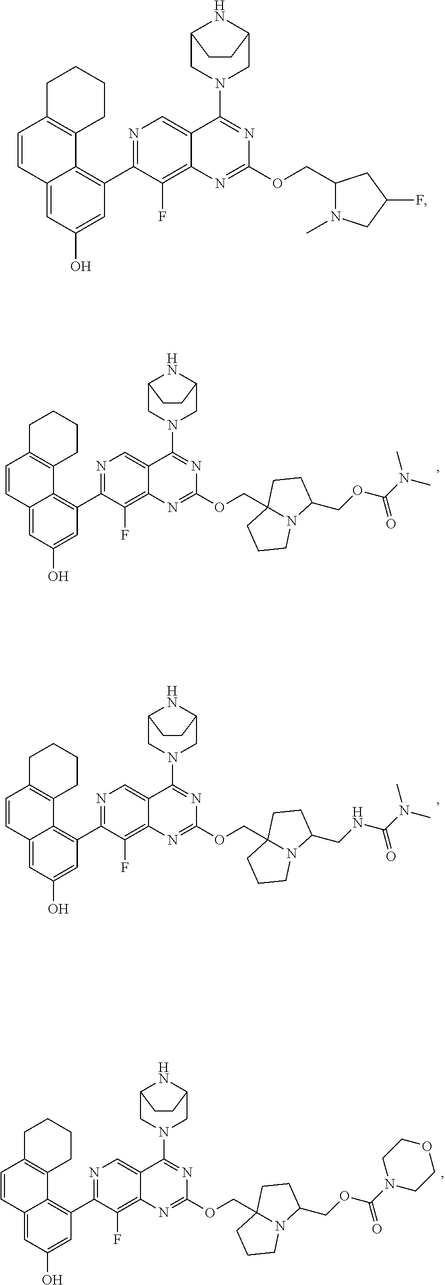 [SMILES: CN1CC(F)CC1COc1nc(N2CC3CCC(C2)N3)c2cnc(-c3cc(O)cc4ccc5c(c34)CCCC5)c(F)c2n1 ; CN(C)C(=O)OCC1CCC2(COc3nc(N4CC5CCC(C4)N5)c4cnc(-c5cc(O)cc6ccc7c(c56)CCCC7)c(F)c4n3)CCCN12 ; CN(C)C(=O)NCC1CCC2(COc3nc(N4CC5CCC(C4)N5)c4cnc(-c5cc(O)cc6ccc7c(c56)CCCC7)c(F)c4n3)CCCN12 ; O=C(OCC1CCC2(COc3nc(N4CC5CCC(C4)N5)c4cnc(-c5cc(O)cc6ccc7c(c56)CCCC7)c(F)c4n3)CCCN12)N1CCOCC1]

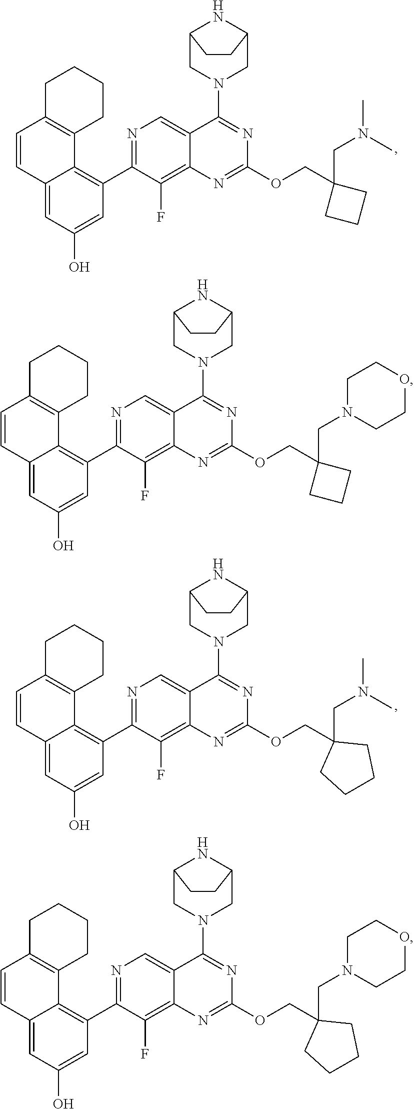 [SMILES: CN(C)CC1(COc2nc(N3CC4CCC(C3)N4)c3cnc(-c4cc(O)cc5ccc6c(c45)CCCC6)c(F)c3n2)CCC1 ; Oc1cc(-c2ncc3c(N4CC5CCC(C4)N5)nc(OCC4(CN5CCOCC5)CCC4)nc3c2F)c2c3c(ccc2c1)CCCC3 ; CN(C)CC1(COc2nc(N3CC4CCC(C3)N4)c3cnc(-c4cc(O)cc5ccc6c(c45)CCCC6)c(F)c3n2)CCCC1 ; Oc1cc(-c2ncc3c(N4CC5CCC(C4)N5)nc(OCC4(CN5CCOCC5)CCCC4)nc3c2F)c2c3c(ccc2c1)CCCC3]

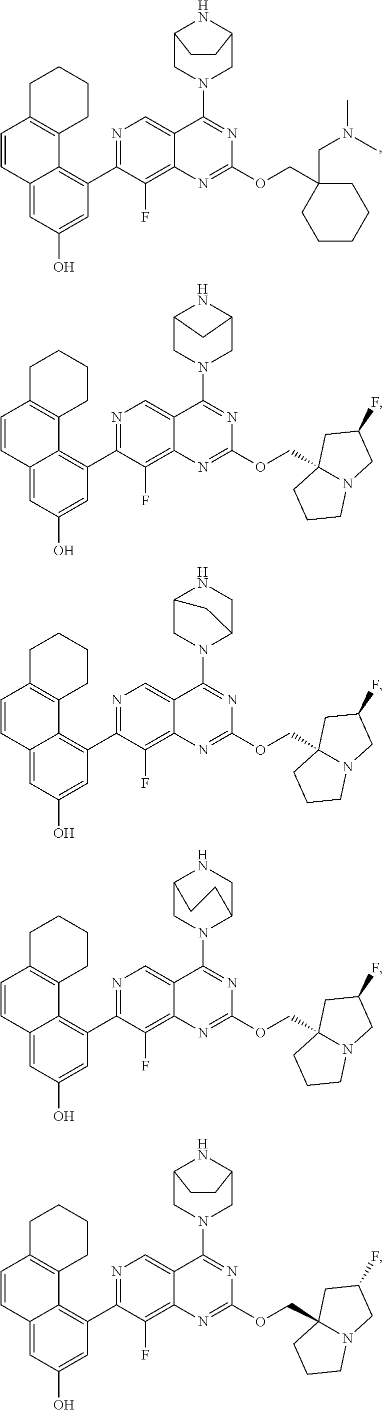 [SMILES: CN(C)CC1(COc2nc(N3CC4CCC(C3)N4)c3cnc(-c4cc(O)cc5ccc6c(c45)CCCC6)c(F)c3n2)CCCCC1 ; Oc1cc(-c2ncc3c(N4CC5CC(C4)N5)nc(OC[C@@]45CCCN4C[C@H](F)C5)nc3c2F)c2c3c(ccc2c1)CCCC3 ; Oc1cc(-c2ncc3c(N4CC5CC4CN5)nc(OC[C@@]45CCCN4C[C@H](F)C5)nc3c2F)c2c3c(ccc2c1)CCCC3 ; Oc1cc(-c2ncc3c(N4CC5CCC4CN5)nc(OC[C@@]45CCCN4C[C@H](F)C5)nc3c2F)c2c3c(ccc2c1)CCCC3 ; Oc1cc(-c2ncc3c(N4CC5CCC(C4)N5)nc(OC[C@]45CCCN4C[C@@H](F)C5)nc3c2F)c2c3c(ccc2c1)CCCC3]

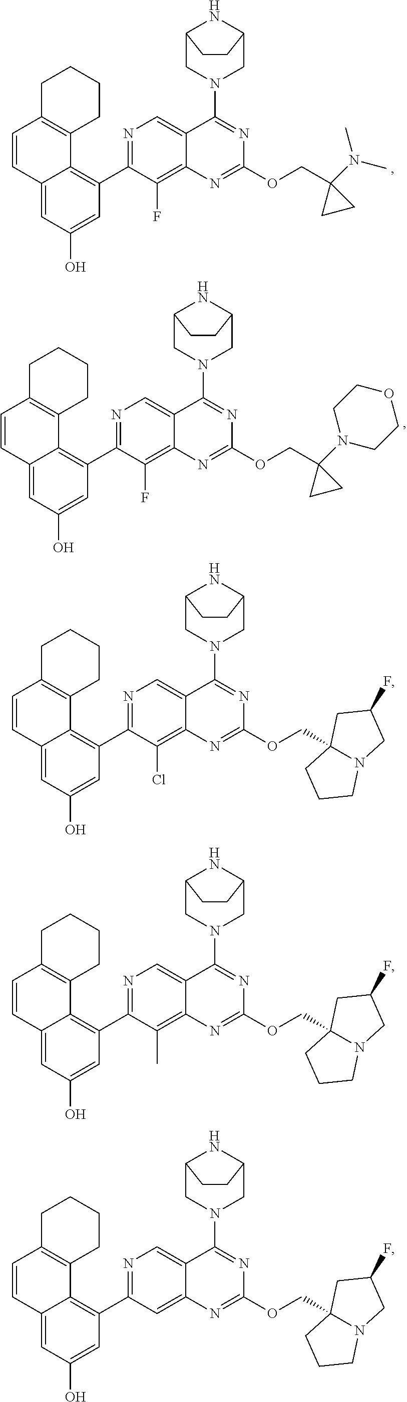 [SMILES: CN(C)C1(COc2nc(N3CC4CCC(C3)N4)c3cnc(-c4cc(O)cc5ccc6c(c45)CCCC6)c(F)c3n2)CC1 ; Oc1cc(-c2ncc3c(N4CC5CCC(C4)N5)nc(OCC4(N5CCOCC5)CC4)nc3c2F)c2c3c(ccc2c1)CCCC3 ; Oc1cc(-c2ncc3c(N4CC5CCC(C4)N5)nc(OC[C@@]45CCCN4C[C@H](F)C5)nc3c2Cl)c2c3c(ccc2c1)CCCC3 ; Cc1c(-c2cc(O)cc3ccc4c(c23)CCCC4)ncc2c(N3CC4CCC(C3)N4)nc(OC[C@@]34CCCN3C[C@H](F)C4)nc12 ; Oc1cc(-c2cc3nc(OC[C@@]45CCCN4C[C@H](F)C5)nc(N4CC5CCC(C4)N5)c3cn2)c2c3c(ccc2c1)CCCC3]

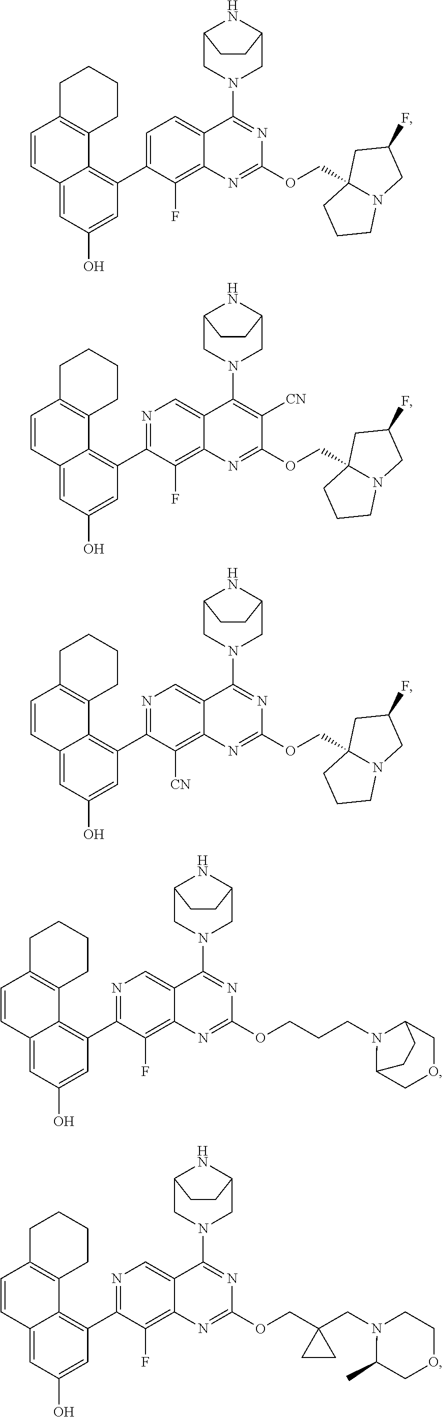 [SMILES: Oc1cc(-c2ccc3c(N4CC5CCC(C4)N5)nc(OC[C@@]45CCCN4C[C@H](F)C5)nc3c2F)c2c3c(ccc2c1)CCCC3 ; N#Cc1c(OC[C@@]23CCCN2C[C@H](F)C3)nc2c(F)c(-c3cc(O)cc4ccc5c(c34)CCCC5)ncc2c1N1CC2CCC(C1)N2 ; N#Cc1c(-c2cc(O)cc3ccc4c(c23)CCCC4)ncc2c(N3CC4CCC(C3)N4)nc(OC[C@@]34CCCN3C[C@H](F)C4)nc12 ; Oc1cc(-c2ncc3c(N4CC5CCC(C4)N5)nc(OCCCN4C5CCC4COC5)nc3c2F)c2c3c(ccc2c1)CCCC3 ; C[C@@H]1COCCN1CC1(COc2nc(N3CC4CCC(C3)N4)c3cnc(-c4cc(O)cc5ccc6c(c45)CCCC6)c(F)c3n2)CC1]

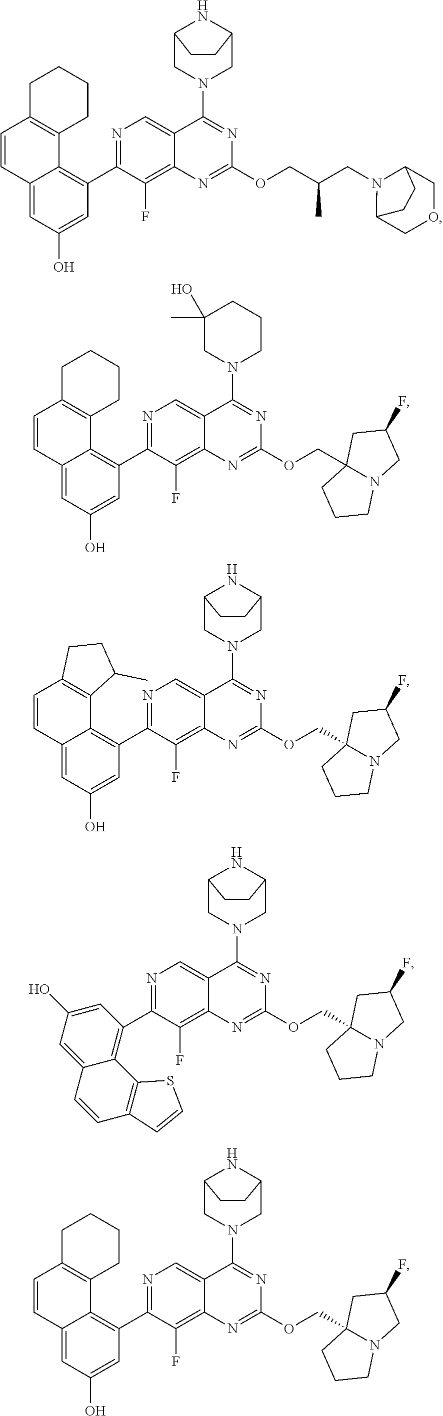 [SMILES: C[C@@H](COc1nc(N2CC3CCC(C2)N3)c2cnc(-c3cc(O)cc4ccc5c(c34)CCCC5)c(F)c2n1)CN1C2CCC1COC2 ; CC1(O)CCCN(c2nc(OCC34CCCN3C[C@H](F)C4)nc3c(F)c(-c4cc(O)cc5ccc6c(c45)CCCC6)ncc23)C1 ; CC1CCc2ccc3cc(O)cc(-c4ncc5c(N6CC7CCC(C6)N7)nc(OC[C@@]67CCCN6C[C@H](F)C7)nc5c4F)c3c21 ; Oc1cc(-c2ncc3c(N4CC5CCC(C4)N5)nc(OC[C@@]45CCCN4C[C@H](F)C5)nc3c2F)c2c(ccc3ccsc32)c1 ; Oc1cc(-c2ncc3c(N4CC5CCC(C4)N5)nc(OC[C@@]45CCCN4C[C@H](F)C5)nc3c2F)c2c3c(ccc2c1)CCCC3]

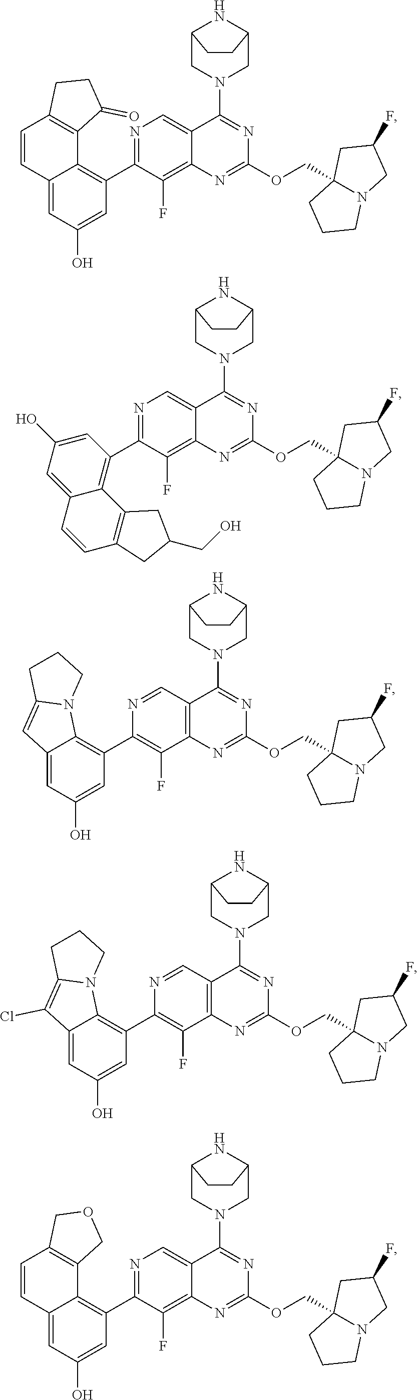

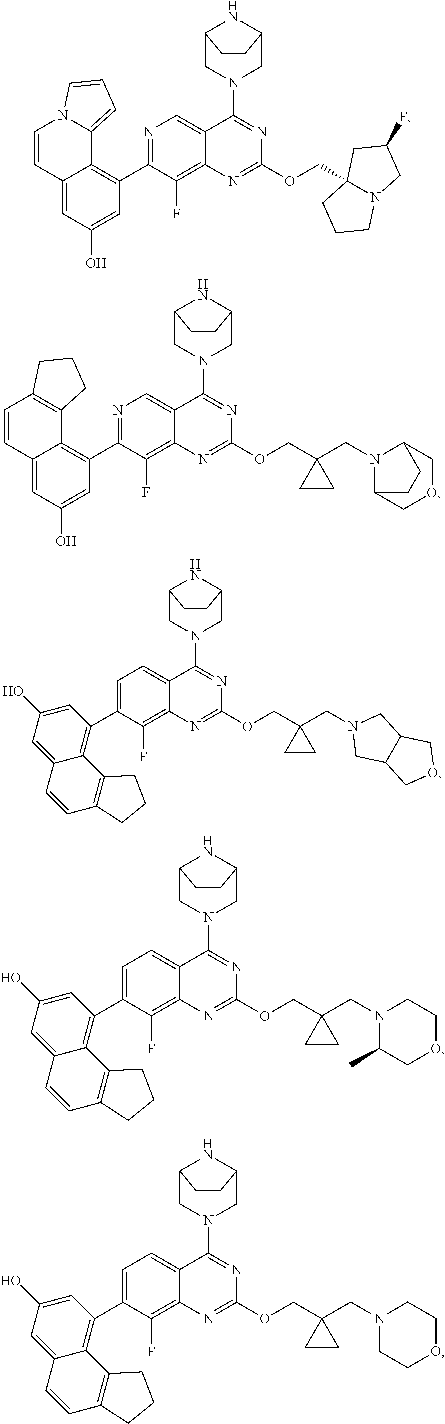 [SMILES: Oc1cc(-c2ncc3c(N4CC5CCC(C4)N5)nc(OC[C@@]45CCCN4C[C@H](F)C5)nc3c2F)c2c(ccn3cccc23)c1 ; Oc1cc(-c2ncc3c(N4CC5CCC(C4)N5)nc(OCC4(CN5C6CCC5COC6)CC4)nc3c2F)c2c3c(ccc2c1)CCC3 ; Oc1cc(-c2ccc3c(N4CC5CCC(C4)N5)nc(OCC4(CN5CC6COCC6C5)CC4)nc3c2F)c2c3c(ccc2c1)CCC3 ; C[C@@H]1COCCN1CC1(COc2nc(N3CC4CCC(C3)N4)c3ccc(-c4cc(O)cc5ccc6c(c45)CCC6)c(F)c3n2)CC1 ; Oc1cc(-c2ccc3c(N4CC5CCC(C4)N5)nc(OCC4(CN5CCOCC5)CC4)nc3c2F)c2c3c(ccc2c1)CCC3]

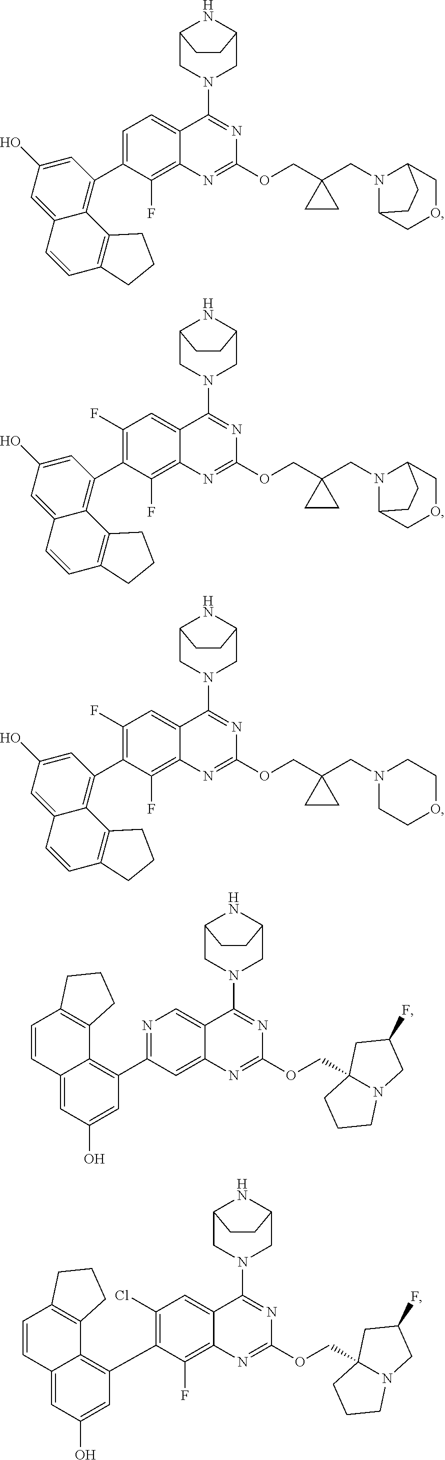 [SMILES: Oc1cc(-c2ccc3c(N4CC5CCC(C4)N5)nc(OCC4(CN5C6CCC5COC6)CC4)nc3c2F)c2c3c(ccc2c1)CCC3 ; Oc1cc(-c2c(F)cc3c(N4CC5CCC(C4)N5)nc(OCC4(CN5C6CCC5COC6)CC4)nc3c2F)c2c3c(ccc2c1)CCC3 ; Oc1cc(-c2c(F)cc3c(N4CC5CCC(C4)N5)nc(OCC4(CN5CCOCC5)CC4)nc3c2F)c2c3c(ccc2c1)CCC3 ; Oc1cc(-c2cc3nc(OC[C@@]45CCCN4C[C@H](F)C5)nc(N4CC5CCC(C4)N5)c3cn2)c2c3c(ccc2c1)CCC3 ; Oc1cc(-c2c(Cl)cc3c(N4CC5CCC(C4)N5)nc(OC[C@@]45CCCN4C[C@H](F)C5)nc3c2F)c2c3c(ccc2c1)CCC3]

or a salt or solvate thereof.

The present disclosure encompasses the preparation and use of salts of Compounds of the Disclosure. As used herein, the term “pharmaceutically acceptable salt” refers to salts or zwitterionic forms of Compounds of the Disclosure that are suitable for administration to a subject, e.g., a human. Salts of Compounds of the Disclosure can be prepared during the final isolation and purification of the compounds or separately by reacting the compound with a suitable acid. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Non-limiting examples of salts of Compounds of the Disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerophosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J Pharmaceut. Sci., 93 (3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5 (1):Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

### III. Compositions

In one aspect, the present disclosure provides compositions comprising the compound of the disclosure, or a pharmaceutically acceptable salt or solvate thereof, and a pharmaceutically acceptable carrier.

Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington's Pharmaceutical Sciences, Mack Publishing Co., Easton, PA, 19th ed. 1995. Such carriers enable the active agents to be formulated as tablets, pills, dragees, capsules, liquids, gels, syrups, slurries, suspensions and the like, for oral ingestion by a subject to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

### IV. Methods and Uses

In one aspect, the present disclosure provides a method for inhibiting KRAS G12D activity in a cell, comprising contacting the cell in which inhibition of KRAS G12D activity is desired with an effective amount of a compound of the disclosure, or a pharmaceutically acceptable salt or solvate thereof; or a pharmaceutical composition of the disclosure.

In one embodiment, the contacting is in vitro. In one embodiment, the contacting is in vivo.

As used herein, the term “contacting” refers to the bringing together of indicated moieties in an in vitro system or an in vivo system. For example, “contacting” a KRAS G12D with a compound provided herein includes the administration of a compound provided herein to an individual or patient, such as a human, having KRAS G12D, as well as, for example, introducing a compound provided herein into a sample containing a cellular or purified preparation containing the KRAS G12D.

By negatively modulating the activity of KRAS G12D, the methods described herein are designed to inhibit undesired cellular proliferation resulting from enhanced KRAS G12D activity within the cell. The cells may be contacted in a single dose or multiple doses in accordance with a particular treatment regimen to effect the desired negative modulation of KRAS G12D. The ability of compounds to bind KRAS G12D may be monitored in vitro using well known methods. In addition, the inhibitory activity of exemplary compounds in cells may be monitored, for example, by measuring the inhibition of KRAS G12D activity of the amount of phosphorylated ERK.

In another aspect, the present disclosure provides a method for treating a KRAS G12D-associated cancer comprising administering to a patient in need thereof a therapeutically effective amount of a compound of the disclosure, or a pharmaceutically acceptable salt or solvate thereof; or a pharmaceutical composition of the disclosure.

In another aspect, the present disclosure provides a method for treating cancer in a patient in need thereof, the method comprising (a) determining that the cancer is associated with a KRAS G12D mutation (e.g., a KRAS G12D-associated cancer); and (b) administering to the patient a therapeutically effective amount of a compound of he disclosure, or a pharmaceutically acceptable salt or solvate thereof; or a pharmaceutical composition of the disclosure.

The compounds and compositions provided herein may be used for the treatment of a wide variety of cancers including tumors such as lung, prostate, breast, brain, skin, cervical carcinomas, testicular carcinomas, etc. More particularly, cancers that may be treated by the compounds and compositions of the disclosure include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

In certain embodiments, the cancer is non-small cell lung cancer, small cell lung cancer, colorectal cancer, rectal cancer or pancreatic cancer. In certain embodiments, the cancer is non-small cell lung cancer.

A therapeutically effective amount of a Compound of the Disclosure required for use in therapy varies with the nature of the condition being treated, the length of time that activity is desired, and the age and the condition of the subject, and ultimately is determined by the attendant physician. Dosage amounts and intervals can be adjusted individually to provide plasma levels of the Compound of the Disclosure that are sufficient to maintain the desired therapeutic effects. The desired dose can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, about 0.05, about 0.5, about 5, about 10, about 20, about 30, about 40, about 50, about 100, about 150, about 200, about 250, about 300, about 350, about 400, about 450, or about 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual subject, which can vary with the age, weight, and response of the particular subject.

In certain embodiments, the therapeutically effective amount of the compound is between about 0.01 to 100 mg/kg per day.

In another aspect, the present disclosure provides compound of the disclosure, or a pharmaceutically acceptable salt or solvate thereof, for use in inhibiting KRAS G12D activity in a cell.

In another aspect, the present disclosure provides compound of the disclosure, or a pharmaceutically acceptable salt or solvate thereof, for use in treating a KRAS G12D-associated cancer.

In another aspect, the present disclosure provides the use of the compound of the disclosure, or a pharmaceutically acceptable salt or solvate thereof, or the pharmaceutical composition of the disclosure, in the manufacture of a medicament for inhibiting KRAS G12D activity in a cell.

In another aspect, the present disclosure provides the use of the compound of the disclosure, or a pharmaceutically acceptable salt or solvate thereof, or the pharmaceutical composition of the disclosure, in the manufacture of a medicament for treating a KRAS G12D-associated cancer.

### V. Kits

In another embodiment, the present disclosure provides kits which comprise a Compound of the Disclosure (or a composition comprising a Compound of the Disclosure) packaged in a manner that facilitates their use to practice methods of the present disclosure. In one embodiment, the kit comprises Compound of the Disclosure (or a composition comprising a Compound of the Disclosure), and instructions for administering the compound, or a pharmaceutically acceptable salt or solvate thereof, to a subject having cancer. In one embodiment, the compound or composition is packaged in a unit dosage form. The kit further can include a device suitable for administering the composition according to the intended route of administration.

### EXAMPLES

In order to make the objects and technical solutions of the present disclosure clearer, the present disclosure will be further described below in conjunction with specific example. It should be understood that the examples are not intended to limit the scope of the invention. Further, specific experimental methods not mentioned in the following examples were carried out in accordance with a conventional experimental method.

### Synthesis of Intermediate

### Intermediate 1:

### 2-(7-(methoxymethoxy)naphtho[1,2-b]furan-9-yl)-4,4,5,5-tetramethyl-1,3,2-dioxaborolane

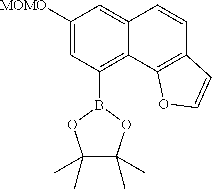

### Step 1: 2,6-dibromo-4-methoxyaniline

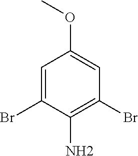

Under Ar, to a solution of 4-methoxyaniline (10.0 g, 81 mmol) in the mixture of DCM (100 mL)/MeOH (100 mL) was added Br₂ (27.2 g, 171 mmol) at 0° C. The reaction was stirred at room temperature for 16 hours. Quenched with Na₂S₂O₃ solution and the resulting mixture was extracted with DCM three times. The combined organic layers were washed with brine, dried over Na₂SO₄ and concentrated in vacuo to give a residual, which was purified by silica gel column and eluted with EA/Hex from 0% to 10% to afford the title compound (12.0 g, 52.6%) as a white solid. MS: 280.0, 282.0, 283.9 (M+H⁺).

### Step 2: 2-bromo-4-methoxy-6-((trimethylsilyl)ethynyl) aniline

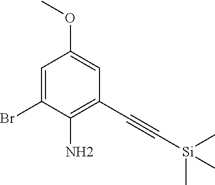 [SMILES: COc1cc(Br)c(N)c(C#C[Si](C)(C)C)c1]

The mixture of CuI (0.1 g, 0.534 mmol) and Pd(PPh₃)₂Cl₂ (0.375 g, 0.5341 mmol) in round-bottom flask was degassed under high vacuum and back-filled with nitrogen, which process was repeated three times, then a solution of 2,6-dibromo-4-methoxyaniline (6.0 g, 21.36 mmol), ethynyltrimethylsilane (2.1 g, 21.36 mmol) and and TEA (20 mL, 143 mmol) in THF (60 mL) was added. The reaction mixture was stirred at 80° C. for 2 hours. After cooling down to room temperature, water was added and the resulting mixture was extracted with EA for three times. The combined organic layers were washed with brine, dried over Na₂SO₄ and concentrated in vacuo to give a residual, which was purified by silica gel column and eluted with EA/Hex from 0% to 5% to afford the title compound (5.0 g, 78%) as yellow oil. MS: 298.2, 300.2 (M+H)⁺

### Step 3: ((3-bromo-2-iodo-5-methoxyphenyl) ethynyl) trimethylsilane

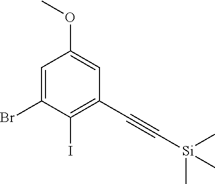 [SMILES: COc1cc(Br)c(I)c(C#C[Si](C)(C)C)c1]

To a solution of 2-bromo-4-methoxy-6-((trimethylsilyl)ethynyl) aniline (4.0 g, 13.41 mmol) and 4-methylbenzenesulfonic acid (7.39 g, 42.9 mmol) in ACN/H₂O=20:1 (100 mL/10 mL) was added sodium nitrite (2.96 g, 42.9 mmol) and potassium iodide (6.68 g, 40.2 mmol) at −5-−10° C. The reaction mixture was stirred at rt for 24 h. Water was added to the mixture and the resulting mixture was extracted with EA for 3 times. The combined organic layers were washed with Na₂S₂O₃ aq, brine, dried over anhydrous Na₂SO₄ and concentrated in vacuo to give a residual, which was purified by silica gel column and eluted with PE to give the title compound (1.15 g, 21%) as colorless oil.

¹H NMR (400 MHz, DMSO) δ 7.16 (d, J=3.2 Hz, 1H), 6.96 (d, J=3.2 Hz, 1H), 3.78 (s, 3H), 0.28 (s, 9H).

### Step 4: ((3-bromo-2-(furan-2-yl)-5-methoxyphenyl) ethynyl) trimethylsilane

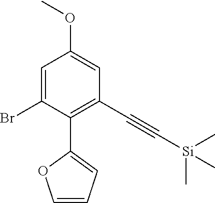 [SMILES: COc1cc(Br)c(-c2ccco2)c(C#C[Si](C)(C)C)c1]

Under Ar, the mixture of ((3-bromo-2-iodo-5-methoxyphenyl) ethynyl) trimethylsilane (1.15 g, 2.81 mmol), PdCl₂(dppf) (206 mg, 0.281 mmol), Na₂CO₃ (1.49 g, 14.1 mmol) and furan-2-ylboronic acid (340 mg, 3.04 mmol) in Dioxane (20 mL) and water (5 mL) was stirred at 100° C. for 3 h. After cooling down to room temperature, water was added, extracted with EA for 3 times. The combined organic layers were washed with brine, dried over anhydrous Na₂SO₄ and concentrated in vacuo to give a residual, which was purified by silica gel column and eluted with PE/MTBE=30:1 to afford the title compound (400 mg, 40.7%) as yellow oil. MS: 349.2 (M+H⁺)

### Step 5: 2-(2-bromo-6-ethynyl-4-methoxyphenyl) furan

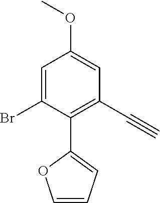 [SMILES: C#Cc1cc(OC)cc(Br)c1-c1ccco1]

Under Ar, the mixture of ((3-bromo-2-(furan-2-yl)-5-methoxyphenyl) ethynyl) trimethylsilane (400 mg, 1.145 mmol) and K₂CO₃ (475 mg, 3.44 mmol) in MeOH (10 mL) was stirred at rt for 1 h. The filtrate was concentrated in vacuo to give a residual, which was purified by silica gel column and eluted with Hex/MTBE=50:1 to afford the title compound (280 mg, 88%) as a yellow oil.

¹H NMR (400 MHz, CDCl₃) δ 7.54 (m, 1H), 7.21 (d, J=2.8 Hz, 1H), 7.06 (d, J=2.8 Hz, 1H), 6.55-6.53 (m, 1H), 6.52-6.50 (m, 1H), 3.83 (s, 3H), 3.06 (s, 1H).

### Step 6: 9-bromo-7-methoxynaphtho[1,2-b]furan

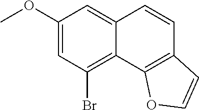 [SMILES: COc1cc(Br)c2c(ccc3ccoc32)c1]

Under Ar, the mixture of 2-(2-bromo-6-ethynyl-4-methoxyphenyl) furan (280 mg, 1.01 mmol) and PtCl₂ (40 mg, 015 mmol) in acetone (20 mL) was refluxed for 8 hours. After the volatiles were under reduced pressure to give a residual, which was purified by silica gel column and eluted with Hex and MTBE from 0% to 1% to afford the title compound (120 mg, 43%) as a white solid.

¹H NMR (400 MHz, CDCl₃) δ 7.82 (d, J=2.0 Hz, 1H), 7.67 (d, J=8.4 Hz, 1H), 7.58 (d, J=8.4 Hz, 1H), 7.56 (d, J=2.8 Hz, 1H), 7.25 (d, J=2.8 Hz, 1H), 6.90 (d, J=2.0 Hz, 1H), 3.93 (s, 3H).

### Step 7: 9-bromonaphtho[1,2-b]furan-7-ol

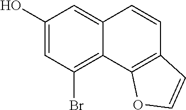 [SMILES: Oc1cc(Br)c2c(ccc3ccoc32)c1]

Under Ar, to a solution of 9-bromo-7-methoxynaphtho[1,2-b]furan (120 mg, 0.433 mmol) in dry DCM (5 mL) was added BBr₃ (2.16 mL, 2.16 mmol, 1M in DCM) at 0° C., then the reaction mixture was stirred at rt for 2 h. Quenched with water, extracted with DCM twice. The combined organic layers were washed with brine, dried over anhydrous Na₂SO₄ and concentrated in vacuo to give a residual, which was purified by silica gel column and eluted with Hex/EA=5:1 to afford the title compound (50 mg, 44%) as yellow oil. MS: 263.1 (M+H⁺)

### Step 8: 9-bromo-7-(methoxymethoxy) naphtho[1,2-b]furan

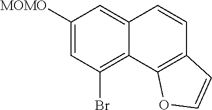

Under Ar, to a solution of 9-bromonaphtho[1,2-b]furan-7-ol (50 mg, 0.19 mmol) and DIPEA (49 mg, 0.38 mmol) in dry DCM (5 mL) was added bromo(methoxy)methane (36 mg, 0.285 mmol) at 0° C. After stirred at rt for 30 mins. Water was added, the resulting mixture was extracted with DCM twice, the combined organic layers were washed with brine, dried over anhydrous Na₂SO₄ and concentrated in vacuo to give a residual, which was purified by silica gel column and eluted with Hex/EA=20:1 to afford the title compound (50 mg, 86%) as yellow oil. MS: 307.4 (M+H⁺)

### Step 9: 2-(7-(methoxymethoxy)naphtho[1,2-b]furan-9-yl)-4,4,5,5-tetramethyl-1,3,2-dioxaborolane

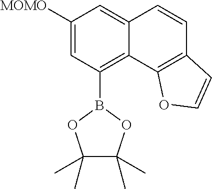

Under Ar, the mixture of 9-bromo-7-(methoxymethoxy)naphtho[1,2-b]furan (40 mg, 0.13 mmol), 4,4,4′,4′,5,5,5′,5′-octamethyl-2,2′-bi(1,3,2-dioxaborolane) (50 mg, 0.195 mmol), potassium acetate (38 mg, 0.39 mmol) and PdCl₂(dppf) (10 mg, 0.013 mmol) in Dioxane (5 mL) was stirred at 100° C. for 2 hours. After cooling down to room temperature, water was added, extracted with EA for 3 times. The combined EA layers were washed with brine, dried over anhydrous Na₂SO₄ and concentrated in vacuo to give a residual, which was purified by silica gel column and eluted with Hex/EA=20:1 to afford the title compound (30 mg, 65%) as colorless oil. MS: 355.6 (M+H⁺)

### Intermediate 2:

### 2-(7-(methoxymethoxy)naphtho[1,2-b]furan-9-yl)-4,4,5,5-tetramethyl-1,3,2-dioxaborolane

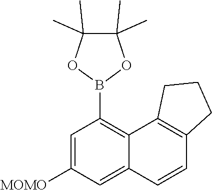

### Step 1: ((3-bromo-2-(cyclopent-1-en-1-yl)-5-methoxyphenyl)ethynyl)trimethyl silane

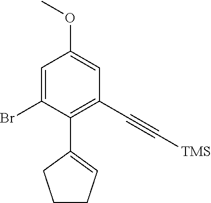 [SMILES: COc1cc(Br)c(C2=CCCC2)c(C#C[Si](C)(C)C)c1]

Under Ar, the mixture of ((3-bromo-2-iodo-5-methoxyphenyl)ethynyl)trimethyl silane (600 mg, 1.467 mmol, Intermediate 1, step 3), 2-(cyclopent-1-en-1-yl)-4,4,5,5-tetramethyl-1,3,2-dioxaborolane (285 mg, 1.467 mmol), Na₂CO₃ (777 mg, 7.33 mmol) and Pd(dppf)Cl₂ (106 mg, 0.147 mmol) in 1,4-Dioxane (20 mL) and Water (5 mL) was stirred at 100° C. for 3 hours. After cooling down to room temperature, water was added, the resulting mixture was extracted with EA for 3 times. The combined organic layers were washed with brine, dried over anhydrous Na₂SO₄ and concentrated in vacuo to give a residual, which was purified by silica gel column and eluted with PE to afford the title compound (260 mg, 50.8%) as yellow oil. ¹H NMR (400 MHz, CDCl₃) δ7.10 (d, J=2.8 Hz, 1H), 6.94 (d, J=2.8 Hz, 1H), 5.69-5.67 (m, 1H), 3.78 (s, 3H), 2.64-2.60 (m, 2H), 2.54-2.49 (m, 2H), 2.06-1.98 (m, 2H), 0.21 (s, 9H).

### Step 2: 1-bromo-2-(cyclopent-1-en-1-yl)-3-ethynyl-5-methoxybenzene

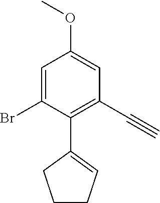 [SMILES: C#Cc1cc(OC)cc(Br)c1C1=CCCC1]

Under Ar, the mixture of ((3-bromo-2-(cyclopent-1-en-1-yl)-5-methoxyphenyl) ethynyl)trimethylsilane (260 mg, 0.744 mmol), and K₂CO₃ (309 mg, 2.233 mmol) in MeOH (20 mL) was stirred at rt for 1 h. The filtrate was concentrated in vacuo to give a residual, which was purified by silica gel column and eluted with PE/EA=10:1 to give the title compound (163 mg, 79%) as a brown oil. MS: 277.2 (M+H⁺).

### Step 3: 9-bromo-7-methoxy-2,3-dihydro-1H-cyclopenta[a]naphthalene

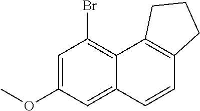 [SMILES: COc1cc(Br)c2c3c(ccc2c1)CCC3]

Under Ar, the mixture of 1-bromo-2-(cyclopent-1-en-1-yl)-3-ethynyl-5-methoxy benzene (163 mg, 0.588 mmol) and platinum(II) chloride (50 mg, 0.188 mmol) in acetone (5 mL) was stirred at refluxing overnight. After the removal off the volatiles under reduced pressure, the residual was purified by silica gel column and eluted with PE to afford the title compound (68 mg, 41.7%) as a yellow solid. ¹H NMR (400 MHz, CDCl₃) δ7.55 (d, J=8.4 Hz, 1H), 7.47 (d, J=2.8 Hz, 1H), 7.34 (d, J=8.4 Hz, 1H), 7.10 (d, J=2.4 Hz, 1H), 3.88 (s, 3H), 3.82 (t, J=7.6 Hz, 2H), 3.00 (t, J=7.6 Hz, 2H), 2.18-2.10 (m, 2H). MS: 277.2 (M+H⁺).

### Step 4: 9-bromo-2,3-dihydro-1H-cyclopenta[a]naphthalen-7-ol

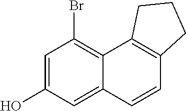 [SMILES: Oc1cc(Br)c2c3c(ccc2c1)CCC3]

Under Ar, to a solution of 9-bromo-7-methoxy-2,3-dihydro-1H-cyclopenta[a]naphthalene (68 mg, 0.245 mmol) in dry DCM was added tribromoborane (307 mg, 1.225 mmol) at ice-water condition, then then mixture was stirred at rt for 2 hours. Water was added, the resulting mixture was extracted with DCM for 3 times. The combined organic layers were washed with brine, dried over anhydrous Na₂SO₄ and concentrated in vacuo to give a residual, which was purified by silica gel column and eluted with Hex/EA=4:1 to afford the title compound (58 mg, 90%) as a white solid.

MS: 263.2 (M+H)+.

### Step 5: 9-bromo-7-(methoxymethoxy)-2,3-dihydro-1H-cyclopenta[a]naphthalene

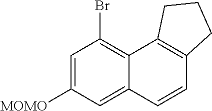

Under Ar, to a solution of 9-bromo-2,3-dihydro-1H-cyclopenta[a]naphthalen-7-ol (58 mg, 0.220 mmol) and DIPEA (142 mg, 1.102 mmol) in dry DCM (5 ml) was added bromo(methoxy)methane at 0° C. After the mixture was stirred at room temperature for 30 mins, water was added. Extracted with DCM for 3 times. The combined organic layers were washed with brine, dried over anhydrous Na₂SO₄ and concentrated in vacuo to give a residual, which was purified by silica gel column and eluted with Hex/EA=20:1 to afford the title compound (62 mg, 92%) as a white solid. MS: 307.2 (M+H⁺)

### Step 6: 2-(7-(methoxymethoxy)-2,3-dihydro-1H-cyclopenta[a]naphthalen-9-yl)-4,4,5,5-tetramethyl-1,3,2-dioxaborolane

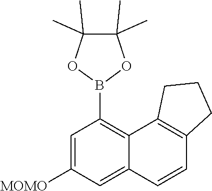

Under Ar, the mixture of 9-bromo-7-(methoxymethoxy)-2,3-dihydro-1H-cyclo penta[a]naphthalene (62 mg, 0.202 mmol), 4,4,4′,4′,5,5,5′,5′-octamethyl-2,2′-bi(1,3,2-dioxaborolane) (77 mg, 0.303 mmol), potassium acetate (59.4 mg, 0.605 mmol) and Pd(dppf)Cl₂ (14.77 mg, 0.020 mmol) in 1,4-Dioxane (3 mL) was stirred at 100° C. for 2 hours. The volatiles were removed under reduced pressure to give a residual, which was purified by silica gel column and eluted with Hex/EA=20:1 to afford the title compound (40 mg, 55.9%) as colorless oil. MS: 355.3 (M+H⁺)

### Intermediate 3:

### 2-(1,2-dihydronaphtho[2,1-b]furan-9-yl)-4,4,5,5-tetramethyl-1,3,2-dioxaborolane

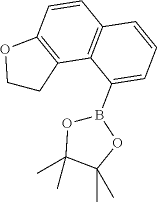 [SMILES: CC1(C)OB(c2cccc3ccc4c(c23)CCO4)OC1(C)C]

### Step 1: 9-bromo-1,2-dihydronaphtho[2,1-b]furan

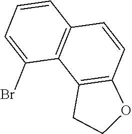 [SMILES: Brc1cccc2ccc3c(c12)CCO3]

Under Ar, to a solution of 8-bromonaphthalen-2-ol (500 mg, 2.241 mmol) and (2-bromoethyl)diphenylsulfonium trifluoromethanesulfonate (1490 mg, 3.36 mmol) in Acetonitrile (10 mL) was added K₂CO₃ (929 mg, 6.72 mmol) at ice-water condition, then the mixture was stirred at rt overnight. Water was added, the resulting mixture was extracted with EA for 3 times. The combined organic layers were washed with brine, dried over sodium sulfate, filtered, concentrated in vacuo to give a residual, which was purified by silica gel column and eluted with ethyl acetate/hexane from 0% to 10% to afford the title compound (380 mg, 68.1%) as a white solid. ¹HNMR (400 MHz, DMSO-d₆) δ 7.93-7.90 (d, J=8.4 Hz, 1H), 7.87-7.85 (d, J=8.4 Hz, 1H), 7.81-7.79 (d, J=7.2 Hz, 1H), 7.25-7.23 (d, J=8.8 Hz, 1H), 7.21-7.17 (t, J=8.0 Hz, 1H), 4.71-4.67 (t, J=8.8 Hz, 2H), 4.04-3.99 (t, J=8.0 Hz, 2H).

### Step 2: 2-(1,2-dihydronaphtho[2,1-b]furan-9-yl)-4,4,5,5-tetramethyl-1,3,2-dioxa borolane

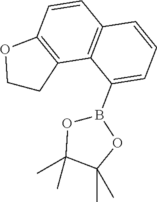 [SMILES: CC1(C)OB(c2cccc3ccc4c(c23)CCO4)OC1(C)C]

Under Ar, the mixture of 9-bromo-1,2-dihydronaphtho[2,1-b]furan (100 mg, 0.401 mmol), PdCl₂(dppf) (29.4 mg, 0.040 mmol), 4,4,4′,4′,5,5,5′,5′-octamethyl-2,2′-bi(1,3,2-dioxaborolane) (153 mg, 0.602 mmol) and potassium acetate (118 mg, 1.204 mmol) in Dioxane (22 mL) was stirred at 85° C. for 4 hours. The volatiles were removed under reduced pressure to give a residual, which was purified by silica gel column and eluted with ethyl acetate/hexane from 0% to 5% to afford the titled compound (80 mg, 67.3%) as a colorless oil. MS: 297.2 (M+H⁺)

### Intermediate 4:

### 4,4,5,5-tetramethyl-2-(naphtho[2,1-b]furan-9-yl)-1,3,2-dioxaborolane

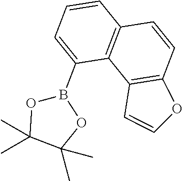 [SMILES: CC1(C)OB(c2cccc3ccc4occc4c23)OC1(C)C]

### Step 1: 9-bromonaphtho[2,1-b]furan

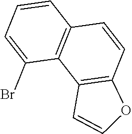 [SMILES: Brc1cccc2ccc3occc3c12]

Under Ar, the mixture of 9-bromo-1,2-dihydronaphtho[2,1-b]furan (72 mg, 0.289 mmol; intermediate 3, step 1) and DDQ (72.2 mg, 0.318 mmol) in toluene (4 mL) was stirred at 110° C. for 16 hs. After cooling down to rt, water was added and the resulting mixture was extracted with EA for 3 times. The combined organic layers were washed with brine, dried over anhydrous sodium sulfate, concentrated in vacuo to give a residual, which was purified by silica gel column and eluted with ethyl acetate/hexane from 0 to 10% to afford the title compound (70 mg, 98%) as a white solid. ¹HNMR (400 MHz, DMSO-d₆) δ 8.24-8.23 (d, J=2.0 Hz, 1H), 8.18-8.17 (d, J=2.0 Hz, 1H), 8.16-8.14 (d, J=8.0 Hz, 1H), 8.06-7.97 (m, 3H), 7.49-7.45 (t, J=8.0 Hz, 1H).

### Step 2: 4,4,5,5-tetramethyl-2-(naphtho[2,1-b]furan-9-yl)-1,3,2-dioxaborolane

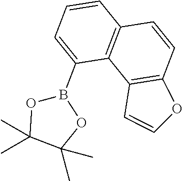 [SMILES: CC1(C)OB(c2cccc3ccc4occc4c23)OC1(C)C]

Under Ar, the mixture of 9-bromonaphtho[2,1-b]furan (70 mg, 0.283 mmol), PdCl₂(dppf) (20.73 mg, 0.028 mmol), 4,4,4′,4′,5,5,5′,5′-octamethyl-2,2′-bi(1,3,2-dioxa borolane) (108 mg, 0.425 mmol) and potassium acetate (83 mg, 0.850 mmol) in Dioxane (22 mL) was stirred at 85° C. for 4 hours. After cooling down to rt, quenched with saturated NH₄Cl solution, extracted with ethyl acetate for 3 times. The combined organic layers were washed with brine, dried over anhydrous sodium sulfate, concentrated in vacuo to give a residual, which was eluted with ethyl acetate/hexane from 0 to 10% to afford the title compound (35 mg, 42.0%) as a white solid. MS: 295.2 (M+H⁺)

### Intermediate 5:

### 2-(7-(methoxymethoxy)naphtho[2,1-b]furan-9-yl)-4,4,5,5-tetramethyl-1,3,2-dioxaborolane

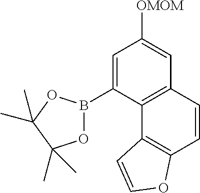

### Step 1: ((3-bromo-2-(furan-3-yl)-5-methoxyphenyl)ethynyl)trimethylsilane

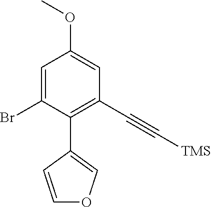 [SMILES: COc1cc(Br)c(-c2ccoc2)c(C#C[Si](C)(C)C)c1]

Under Ar, the mixture of ((3-bromo-2-iodo-5-methoxyphenyl)ethynyl)trimethyl silane (1.5 g, 3.67 mmol), furan-3-ylboronic acid (0.410 g, 3.67 mmol), Na₂CO₃ (0.777 g, 7.33 mmol) and PdCl₂(dppf)-CH₂Cl₂ adduct (0.299 g, 0.367 mmol) in Dioxane (10 mL)/Water (2 mL) was stirred at 90° C. for 2 h. H₂O was added to the reaction mixture, followed by extraction with ethyl acetate three times. The combined organic layers were dried Na₂SO₄, filtered and concentrated in vacuo to give a residual, which was purified by silica gel column and eluted with ethyl acetate/hexane from 0 to 2% to give the title compound (780 mg, 60.9%). MS: 349.2 (M+H⁺).

### Step 2: 3-(2-bromo-6-ethynyl-4-methoxyphenyl)furan

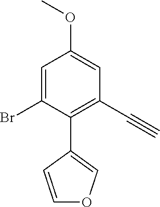 [SMILES: C#Cc1cc(OC)cc(Br)c1-c1ccoc1]

Under Ar, the mixture of ((3-bromo-2-(furan-3-yl)-5-methoxyphenyl)ethynyl) trimethylsilane (780 mg, 2.233 mmol) and K₂CO₃ (617 mg, 4.47 mmol) in MeOH (10 mL) was stirred at RT for 1 h. The filtrate was concentrated in vacuo to give a residual, which was purified by silica gel column and eluted with ethyl acetate/hexane from 0 to 2% to afford the title compound (510 mg, 82%). MS: 277.2 (M+H⁺).

### Step 3: 9-bromo-7-methoxynaphtho[2,1-b]furan

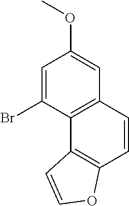 [SMILES: COc1cc(Br)c2c(ccc3occc32)c1]

Under Ar, the mixture of 3-(2-bromo-6-ethynyl-4-methoxyphenyl)furan (510 mg, 1.840 mmol) and platinum(II) chloride (49.0 mg, 0.184 mmol) in Acetone (10 mL) was stirred at 55° C. for 16 hours. After the removal off the volatiles under reduced pressure to afford a residual, which was purified by silica gel column and eluted with ethyl acetate/hexane from 0 to 2% to afford the title compound (195 mg, 38.2%). ¹H NMR (400 MHz, CDCl₃) δ 8.14-8.09 (m, 1H), 7.75 (d, J=2.1 Hz, 1H), 7.69 (d, J=8.9 Hz, 1H), 7.63 (d, J=8.9 Hz, 1H), 7.58 (d, J=2.5 Hz, 1H), 7.28 (d, J=2.4 Hz, 1H), 3.93 (s, 3H). MS: 277.2 (M+H⁺).

### Step 4: 9-bromonaphtho[2,1-b]furan-7-ol

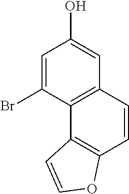 [SMILES: Oc1cc(Br)c2c(ccc3occc32)c1]

Under Ar, to a solution of 9-bromo-7-methoxynaphtho[2,1-b]furan (195 mg, 0.704 mmol) in DCM (5 mL) was added 1M BBr₃ (353 mg, 1.407 mmol) in DCM dropwise at 0° C., and then the mixture was stirred at rt for 1 h. Quenched with water, the resulting mixture was extracted with DCM twice. The combined organic layers were dried Na₂SO₄, filtered and concentrated in vacuo to give a residual, which was purified by silica gel column and eluted with ethyl acetate/hexane from 0 to 20% to give the title compound (128 mg, 69.1%). MS: 263.2 (M+H⁺).

### Step 5:9-bromo-7-(methoxymethoxy)naphtho[2,1-b]furan

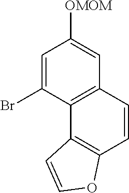

Under Ar, the mixture of 9-bromonaphtho[2,1-b]furan-7-ol (90 mg, 0.342 mmol), DIEA (221 mg, 1.710 mmol) and bromo(methoxy)methane (128 mg, 1.026 mmol) in DCM (5 mL) was stirred at from 0° C. to RT for 1 hour. Water was added, the resulting mixture was extracted with DCM for three times. The combined organic layers were dried over Na₂SO₄, filtered, and concentrated in vacuo to give a residual, which was purified by silica gel column and eluted with ethyl acetate/hexane from 0 to 10% to afford the title compound (83 mg, 79%). MS: 307.4 (M+H⁺).

### Step 6: 2-(7-(methoxymethoxy)naphtho[2,1-b]furan-9-yl)-4,4,5,5-tetramethyl-1,3,2-dioxaborolane

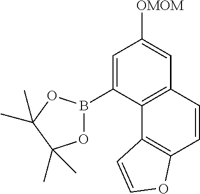

Under Ar, the mixture of 9-bromo-7-(methoxymethoxy)naphtho[2,1-b]furan (83 mg, 0.270 mmol), 4,4,4′,4′,5,5,5′,5′-octamethyl-2,2′-bi(1,3,2-dioxaborolane) (103 mg, 0.405 mmol), potassium acetate (80 mg, 0.811 mmol) and PdCl₂(dppf) (19.77 mg, 0.027 mmol) in Dioxane (5 mL) was stirred at 90° C. for 3 hs. After all the volatiles were removed under reduced pressure to give a residual, which was purified by silica gel column and eluted with ethyl acetate/hexane from 0 to 10% to afford the title compound (65 mg, 67.9%). MS: 355.3 (M+H⁺).

### Intermediate 6:

### 2-(7-(methoxymethoxy)-1,2-dihydronaphtho[2,1-b]furan-9-yl)-4,4,5,5-tetramethyl-1,3,2-dioxaborolane

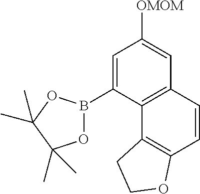

### Step 1: 9-bromo-1,2-dihydronaphtho[2,1-b]furan-7-ol

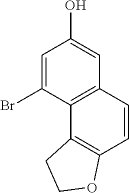 [SMILES: Oc1cc(Br)c2c3c(ccc2c1)OCC3]

Under Ar, to a solution of 9-bromonaphtho[2,1-b]furan-7-ol (128 mg, 0.487 mmol) in TFA (3 mL) was added triethylsilane (283 mg, 2.433 mmol) at ice-water condition, then the mixture was stirred at 50° C. for 20 hours. After the removal off the volatile, saturated NaHCO₃ solution was added. The resulting mixture was extracted with ethyl acetate three times. The combined organic layers were washed with brine, dried over Na₂SO₄, filtered, and concentrated in vacuo to give a residual, which was purified by silica gel column and eluted with ethyl acetate/hexane from 0 to 20% to afford the title compound (120 mg, 93%). ¹H NMR (400 MHz, CDCl₃) δ 7.50 (d, J=8.8 Hz, 1H), 7.39 (d, J=2.4 Hz, 1H), 7.12 (d, J=2.3 Hz, 1H), 7.07 (d, J=8.7 Hz, 1H), 4.65 (t, J=9.1 Hz, 2H), 4.04 (t, J=9.0 Hz, 2H). MS: 265.0 (M+H⁺).

### 2-(7-(methoxymethoxy)-1,2-dihydronaphtho[2,1-b]furan-9-yl)-4,4,5,5-tetramethyl-1,3,2-dioxaborolane

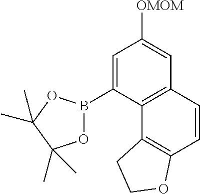

The title intermediate was prepared essentially the same protocol described in intermediate 5.

### Intermediate 7: 2-(7-(methoxymethoxy)-1-methyl-2,3-dihydro-1H-cyclopenta[a]naphthalen-9-yl)-4,4,5,5-tetramethyl-1,3,2-dioxaborolane

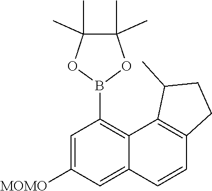

### Step 1: ((3-bromo-2-(hex-1-yn-1-yl)-5-methoxyphenyl)ethynyl)trimethylsilane

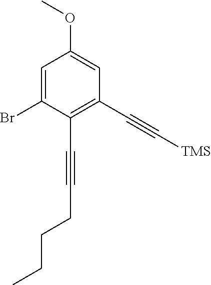 [SMILES: CCCCC#Cc1c(Br)cc(OC)cc1C#C[Si](C)(C)C]

Under Ar, the mixture of ((3-bromo-2-iodo-5-methoxyphenyl)ethynyl)trimethylsilane (4 g, 9.78 mmol), hex-1-yne (1.205 g, 14.67 mmol), Pd(PPh₃)₂Cl₂ (0.343 g, 0.489 mmol), copper(I) iodide (0.186 g, 0.978 mmol) in TEA (10 mL) was stirred at 80° C. for 3 hs. After cooling down to RT, the volatiles was removed under reduced pressure to give a residual, which was purified by silica gel column and elutated with PE to afford the title compound (799 mg, 22.49%) as a brown oil. H NMR (CDCl₃, 400 Hz): 7.09 (d, J=2.4 Hz, 1H), 6.92 (d, J=2.4 Hz, 1H), 3.77 (s, 3H), 2.50 (t, J=7.2 Hz, 2H), 1.66-1.61 (m, 2H), 1.58-1.51 (m, 2H), 0.95 (t, J=7.2 Hz, 3H), 0.26 (s, 9 Hz).

### Step 2: 1-bromo-3-ethynyl-2-(hex-1-yn-1-yl)-5-methoxybenzene

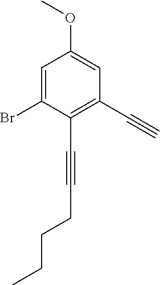 [SMILES: C#Cc1cc(OC)cc(Br)c1C#CCCCC]

Under Ar, the mixture of ((3-bromo-2-(hex-1-yn-1-yl)-5-methoxyphenyl)ethynyl)trimethylsilane (799 mg, 2.199 mmol), K₂CO₃ (912 mg, 6.60 mmol) in MeOH (20 mL) was stirred at RT for 1 h. The volatiles was removed under reduced pressure to give a residual, which was purified by silica gel column and eluted with EA/PE from 0 to 10% to afford the title compound (446 mg, 69.7%) as a brown oil. MS: 293.3 (M+H⁺).

### Step 3: 9-bromo-7-methoxy-1-methyl-2,3-dihydro-1H-cyclopenta[a]naphthalene

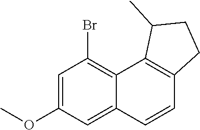 [SMILES: COc1cc(Br)c2c3c(ccc2c1)CCC3C]

Under Ar, the mixture of 1-bromo-3-ethynyl-2-(hex-1-yn-1-yl)-5-methoxybenzene (446 mg, 1.532 mmol), platinum(II) chloride (20.37 mg, 0.077 mmol) in toluene (5 mL) was stirred at 100° C. for 3 days. After the reaction, the volatiles was removed under reduced pressure to afford a residual, which was purified by silica gel column and eluted with PE to afford the title compound (102 mg, 22.87%) as a colorless oil. MS: 293.1 (M+H⁺).

### Step 4: 9-bromo-1-methyl-2,3-dihydro-1H-cyclopenta[a]naphthalen-7-ol

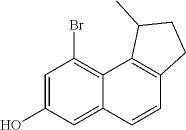 [SMILES: CC1CCc2ccc3cc(O)cc(Br)c3c21]

Under Ar, to a solution of 9-bromo-7-methoxy-1-methyl-2,3-dihydro-1H-cyclopenta[a]naphthalene (106 mg, 0.364 mmol) in dry DCM was added tribromoborane (1893 mg, 7.56 mmol) at 0° C. After stirred at RT for 2 hours, quenched with water and the resulting mixture was extracted with DCM three times. The combined organic layers were washed with brine, dried over anhydrous Na₂SO₄ and concentrated in vacuo to afford a residue, which was purified by silica gel column and eluted with EA/PE from 0 to 25% to afford the title compound (86 mg, 85%) as a colorless oil. MS: 279.2 (M+H⁺).

### Step 5: 9-bromo-7-(methoxymethoxy)-1-methyl-2,3-dihydro-1H-cyclopenta[a]naphthalene

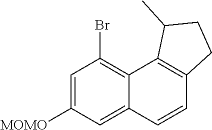

Under Ar, to a solution of 9-bromo-1-methyl-2,3-dihydro-1H-cyclopenta[a]naphthalen-7-ol (86 mg, 0.310 mmol) and DIPEA (201 mg, 1.551 mmol) in dry DCM (5 mL) was added bromo(methoxy)methane at 0° C. The reaction solution was stirred at rt for 30 min. Quenched with water, the resulting mixture was extracted with DCM three times. The combined organic layers were wash with brine, dried over anhydrous Na₂SO₄ and concentrated in vacuo to give a residual, which was purified by silica gel column and eluted with EA/Hex from 0 to 5% to afford the title compound (87 mg, 87%) as a white solid. MS: 321.1 (M+H⁺).

### Step 6: 2-(7-(methoxymethoxy)-1-methyl-2,3-dihydro-1H-cyclopenta[a]naphthalen-9-yl)-4,4,5,5-tetramethyl-1,3,2-dioxaborolane

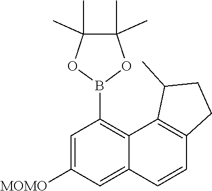

Under Ar, the mixture of 9-bromo-7-(methoxymethoxy)-1-methyl-2,3-dihydro-1H-cyclopenta[a]naphthalene (86 mg, 0.268 mmol), 4,4,4′,4′,5,5,5′,5′-octamethyl-2,2′-bi(1,3,2-dioxaborolane) (102 mg, 0.402 mmol), potassium acetate (79 mg, 0.803 mmol) and PdCl₂(dppf) (19.59 mg, 0.027 mmol) in 1,4-Dioxane (15 mL) was stirred at 100° C. for 2 hours. The volatiles was removed under reduced pressure to afford a residual, which was purified by silica gel column and eluted with EA/Hex from 0 to 5% to afford the title compound (20 mg, 20.28%) as colorless oil. MS: 369.2 (M+H⁺).

### Intermediate 8: 2-(7-(methoxymethoxy)naphtho[1,2-b]thiophen-9-yl)-4,4,5,5-tetramethyl-1,3,2-dioxaborolane

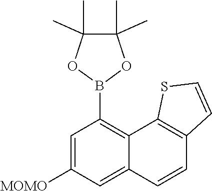

### Step 1: 2-(2-bromo-6-ethynyl-4-methoxyphenyl)thiophene

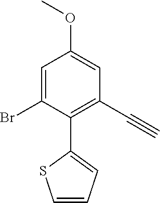 [SMILES: C#Cc1cc(OC)cc(Br)c1-c1cccs1]

Under Ar, the mixture of ((3-bromo-2-iodo-5-methoxyphenyl)ethynyl)trimethylsilane (600 mg, 1.467 mmol), thiophen-2-ylboronic acid (188 mg, 1.467 mmol), sodium carbonate (466 mg, 4.40 mmol) and PdCl₂(dppf) (107 mg, 0.147 mmol) in 1,4-Dioxane (20 mL)/Water (5 mL) was stirred at 100° C. for 3 hours. After cooling down to RT, the mixture was partioned between EA/Water, the separated organic layer was washed with brine, dried over anhydrous Na₂SO₄ and concentrated in vacuo to give a residual, which was purified by silica gel column and eluted with EA/Hex from 0 to 10% to afford the desired compound (130 mg, 30.2%) as brown solid. H NMR (CDCl₃, 400 MHz): 7.43-7.41 (dd, J=4.8 Hz, 0.8 Hz, 1H), 7.23 (d, J=2.8 Hz, 1H), 7.12-7.09 (m, 1H), 7.07 (d, J=2.8 Hz, 1H), 7.03-7.02 (dd, J=4.8 Hz, 0.8 Hz, 1H), 3.83 (s, 3H), 3.02 (s, 1H).

### Step 2: 9-bromo-7-methoxynaphtho[1,2-b]thiophene

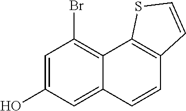 [SMILES: Oc1cc(Br)c2c(ccc3ccsc32)c1]

Under Ar, the mixture of 2-(2-bromo-6-ethynyl-4-methoxyphenyl)thiophene (130 mg, 0.443 mmol), platinum(II) chloride (50 mg, 0.188 mmol) in Acetone (30 mL) was stirred at 60° C. overnight. The volatiles was removed under reduced pressure to give a residual, which was purified by silica gel column and eluted with Hex afford the title compound (72 mg, 55.4%) as a yellow solid. MS: 295.2 (M+H⁺).

### Step 3: 9-bromonaphtho[1,2-b]thiophen-7-ol

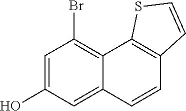 [SMILES: Oc1cc(Br)c2c(ccc3ccsc32)c1]

Under Ar, to a solution of 9-bromo-7-methoxynaphtho[1,2-b]thiophene (72 mg, 0.246 mmol) in dry DCM was added tribromoborane (1893 mg, 7.56 mmol) at 0° C. After stirred at RT for 2 hours, quenched with water, the resulting mixture was extracted with DCM three times. The combined organic layers were washed with brine, dried over anhydrous Na₂SO₄ and concentrated in vacuo to give a residual, which was purified by silica gel column and eluted with EA/Hex from 0 to 25% to afford the desired compound (54 mg, 79%) as a yellow solid. MS: 279.3 (M+H⁺).

### Step 4: 9-bromo-7-(methoxymethoxy)naphtho[1,2-b]thiophene

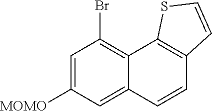

Under Ar, to a solution of 9-bromonaphtho[1,2-b]thiophen-7-ol (54 mg, 0.193 mmol) and DIPEA (75 mg, 0.580 mmol) in dry DCM (2 mL) was added bromo(methoxy)methane at ice-water condition. After stirred at RT for 30 mins. Water was added and the resulting mixture was extracted with DCM for three times. The combined organic layers were washed with brine, dried over anhydrous Na₂SO₄ and concentrated in vacuo to give a residue, which was purified by silica gel column and eluted with EA/Hex from 0 to 10% to afford the desired compound (61 mg, 98%) as a colorless oil. MS: 323.3 (M+H⁺).

### Step 5: 2-(7-(methoxymethoxy)naphtho[1,2-b]thiophen-9-yl)-4,4,5,5-tetramethyl-1,3,2-dioxaborolane

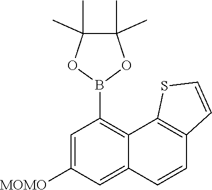

Under Ar, the mixture of 9-bromo-7-(methoxymethoxy)naphtho[1,2-b]thiophene (61 mg, 0.189 mmol), 4,4,4′,4′,5,5,5′,5′-octamethyl-2,2′-bi(1,3,2-dioxaborolane) (71.9 mg, 0.283 mmol), potassium acetate (55.6 mg, 0.566 mmol) and PdCl₂(dppf) (13.81 mg, 0.019 mmol) in 1,4-Dioxane (15 mL) was stirred at 100° C. for 2 h. The volatiles was removed in vacuo to give a residual, which was purified by silica gel column and eluted with EA/Hex from 0 to 5% to afford the title compound (30 mg, 42.9%) as colorless oil. MS: 371.3 (M+H⁺).

### Intermediate 9: 2-(2-(methoxymethoxy)-5,6,7,8-tetrahydrophenanthren-4-yl)-4,4,5,5-tetramethyl-1,3,2-dioxaborolane

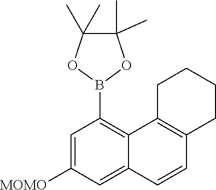

### Step 1: ((6-bromo-4-methoxy-2′,3′,4′,5′-tetrahydro-[1,1′-biphenyl]-2-yl)ethynyl)trimethylsilane

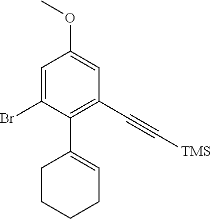 [SMILES: COc1cc(Br)c(C2=CCCCC2)c(C#C[Si](C)(C)C)c1]

Under Ar, the mixture of ((3-bromo-2-iodo-5-methoxyphenyl)ethynyl)trimethylsilane (4 g, 9.78 mmol, 1.000), 2-(cyclohex-1-en-1-yl)-4,4,5,5-tetramethyl-1,3,2-dioxaborolane (2.035 g, 9.78 mmol), Na₂CO₃ (3.11 g, 29.3 mmol) and Pd(dppf)Cl₂ (0.705 g, 0.978 mmol) in 1,4-Dioxane (40 mL) and Water (10 mL) was stirred at 100° C. for 3 hours. After cooling down to RT, the mixture was partioned between EA/Water, the separated organic layer was washed with brine, dried over anhydrous Na₂SO₄ and concentrated in vacuo to give a residue, which was purified by silica gel column and eluted with PE to afford the desired compound (442 mg, 12.44%) as a yellow oil. MS: 363.2 (M+H⁺).

### Step 2: 2′-bromo-6′-ethynyl-4′-methoxy-2,3,4,5-tetrahydro-1,1′-biphenyl

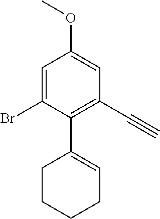 [SMILES: C#Cc1cc(OC)cc(Br)c1C1=CCCCC1]

Under Ar, the mixture of ((6-bromo-4-methoxy-2′,3′,4′,5′-tetrahydro-[1,1′-biphenyl]-2-yl)ethynyl)trimethylsilane (442 mg, 1.216 mmol) and K₂CO₃ (504 mg, 3.65 mmol) in MeOH (20 mL) was stirred at RT for 1 hour. The volatiles was removed under reduced pressure to give a residual, which was purified by silica gel column and eluted with EA/Hex from 0 to 10% to afford the title compound (350 mg, 99%) as a brown oil. MS: 291.2 (M+H⁺).

### Step 3: 5-bromo-7-methoxy-1,2,3,4-tetrahydrophenanthrene

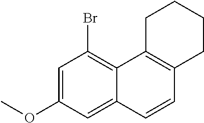 [SMILES: COc1cc(Br)c2c3c(ccc2c1)CCCC3]

Under Ar, the mixture of 2′-bromo-6′-ethynyl-4′-methoxy-2,3,4,5-tetrahydro-1,1′-biphenyl (357 mg, 1.226 mmol), platinum(II) chloride (30 mg, 0.113 mmol) in Acetone (5 mL) was stirred at 60° C. over night. The volatiles was removed under reduced pressure to give a residual, which was purified by silica gel column and eluted with Hex to afford the desired compound (237 mg, 66.4%) as a brown oil. MS: 313.4 (M+Na⁺).

### Step 4: 4-bromo-5,6,7,8-tetrahydrophenanthren-2-ol

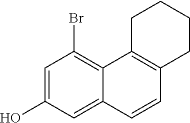 [SMILES: Oc1cc(Br)c2c3c(ccc2c1)CCCC3]

Under Ar, to a solution of 5-bromo-7-methoxy-1,2,3,4-tetrahydrophenanthrene (237 mg, 0.814 mmol) in dry DCM was added tribromoborane (1.02 g, 4.07 mmol) at ice-water condition and then, the mixture was stirred at RT for 2 hours. Quenched with water and the resulting mixture was extracted with DCM three times. The combined organic layers were washed with brine, dried over anhydrous Na₂SO₄ and concentrated in vacuo to give a residue, which was purified by silica gel column and eluted with EA/Hex from 0 to 25% to afford the desired compound (169 mg, 74.9%) as a colorless oil. MS: 277.3 (M+H⁺).

### Step 5: 5-bromo-7-(methoxymethoxy)-1,2,3,4-tetrahydrophenanthrene

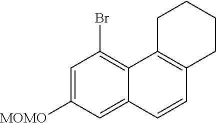

Under Ar, to a solution of 4-bromo-5,6,7,8-tetrahydrophenanthren-2-ol (169 mg, 0.610 mmol) and DIPEA (394 mg, 3.05 mmol) in dry DCM (10 mL) was added bromo(methoxy)methane at ice-water condition. After stirred at RT for 30 min, quenched with water, and the resulting mixture was extracted with DCM for three times, the combined organic layers was washed with brine, dried over anhydrous Na₂SO₄ and concentrated in vacuo to give a residual, which was purified by silica gel column and eluted with EA/Hex from 0 to 5% to afford the desired compound (155 mg, 79%) as a colorless oil. MS: 321.2 (M+H⁺).

### Step 6: 2-(2-(methoxymethoxy)-5,6,7,8-tetrahydrophenanthren-4-yl)-4,4,5,5-tetramethyl-1,3,2-dioxaborolane

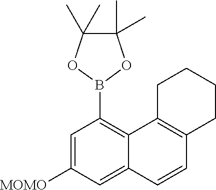

Under Ar, the mixture of 5-bromo-7-(methoxymethoxy)-1,2,3,4-tetrahydrophenanthrene (135 mg, 0.420 mmol) in dry THF (15 mL) was added BuLi (40.4 mg, 0.630 mmol) at −70° C., after the mixture was stirred at −70° C. for 10 mins, 2-isopropoxy-4,4,5,5-tetramethyl-1,3,2-dioxaborolane (1564 mg, 8.41 mmol) was added. At last, the mixture stirred at RT for 30 mins. Quenched with water, the resulting mixture was extracted with EA for three times. The combined organic layers were washed with brine, dried over Na₂SO₄, filtered, concentrated to give a residual, which was purified by silica gel column and eluted with EA/PE from 0 to 5% to afford the desired compound (60 mg, 38.8%) as a colorless oil. MS: 369.3 (M+H⁺).

### Intermediate 10: 7-(methoxymethoxy)-9-(4,4,5,5-tetramethyl-1,3,2-dioxaborolan-2-yl)-2,3-dihydro-1H-cyclopenta[a]naphthalen-1-one

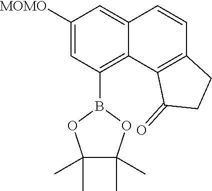

### Step 1: tert-butyl (7-hydroxynaphthalen-1-yl)carbamate

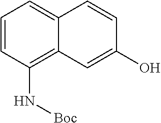 [SMILES: CC(C)(C)OC(=O)Nc1cccc2ccc(O)cc12]

Under Ar, the mixture of 8-aminonaphthalen-2-ol (15 g, 94 mmol) di-tert-butyl dicarbonate (21.59 g, 99 mmol) in DCM (150 mL) and THF (120 mL) was heated to reflux for 36 hours. The volatiles was removed under reduced pressure to afford a residual, which was purified by silica gel column and eluted with EA/DCM from 0 to 10% to afford the desired compound (24 g, 98%) as a brown solid. MS: 282.3 (M+Na⁺).

### Step 2: 8-((tert-butoxycarbonyl)amino)naphthalen-2-yl trifluoromethanesulfonate

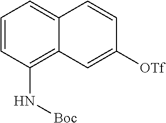 [SMILES: CC(C)(C)OC(=O)Nc1cccc2ccc(OS(=O)(=O)C(F)(F)F)cc12]

Under Ar, to a solution of tert-butyl (7-hydroxynaphthalen-1-yl)carbamate (24 g, 93 mmol) and TEA (30.9 g, 305 mmol) in dichloromethane (20 mL) was added Tf2O (27.7 g, 98 mmol, 1.06) was slowly at −20° C. After the addition, the mixture was stirred at RT for 5 hours. Quenched with ice-cold water and the resulting mixture was extracted with ethyl acetate fro three times. The combined organic layers were washed successively with water, an aqueous solution of sodium hydroxide (1 M) and water, and then dried over MgSO₄ and concentrated in vacuo to give a residual, which was purified by silica gel column and eluted with EA/Hex from 0 to 25% to afford the desired compound (34 g, 94%) as a brown solid. MS: 292.2 (M+H⁺-Boc).

### Step 3: methyl (E)-3-(8-((tert-butoxycarbonyl)amino)naphthalen-2-yl)acrylate

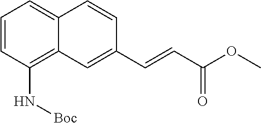 [SMILES: COC(=O)/C=C/c1ccc2cccc(NC(=O)OC(C)(C)C)c2c1]

Under Ar, the mixture of 8-((tert-butoxycarbonyl)amino)naphthalen-2-yl trifluoromethanesulfonate (34 g, 87 mmol), methyl acrylate (22.44 g, 261 mmol), bis(triphenylphosphine)palladium(II) chloride (3.05 g, 4.34 mmol), and TEA (17.58 g, 174 mmol) in DMF (30 mL) was stirred at 90° C. overnight. After cooling to room temperature, water was added and the resulting mixture was extracted with diethyl ether for three times. The combined organic layers were washed with water and brine, dried over magnesium sulphate and concentrated in vacuo to give a, which was purified by silica gel column and eluted with EA/Hex from 0 to 20% to afford the desired compound (31 g, 87%) as a brown solid. MS: 272.2.

### Step 4: methyl (E)-3-(8-aminonaphthalen-2-yl)acrylate

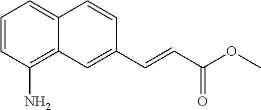 [SMILES: COC(=O)/C=C/c1ccc2cccc(N)c2c1]

Under Ar, the mixture of methyl (E)-3-(8-((tert-butoxycarbonyl)amino)naphthalen-2-yl)acrylate (28 g, 86 mmol) in DCM (100 mL)/TFA (100 mL) was stirred at 20° C. for 4 hours. The volatiles was removed under reduced pressure to give a residual, which was dissolved in EA, and the resulting organic mixture was washed with saturated NaHCO₃, brine, dried over Na₂SO₄, concentrated in vacuo to give a residual, which was purified by silica gel column and eluted with DCM to afford the desired compound (13.7 g, 70.5%) as a brown solid. H NMR (DMSO, 400 MHz): 8.41 (s, 1H), 7.78-7.71 (m, 3H), 7.25 (t, J=8.0 Hz, 1H), 7.06 (d, J=8.0 Hz, 1H), 6.75 (d, J=16 Hz, 1H), 6.68 (d, J=8.0 Hz, 1H), 5.97 (brs, 2H), 3.75 (s, 3H).

### Step 5: methyl 3-(8-aminonaphthalen-2-yl)propanoate

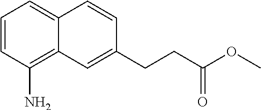 [SMILES: COC(=O)CCc1ccc2cccc(N)c2c1]

The mixture of methyl (E)-3-(8-aminonaphthalen-2-yl)acrylate (13.7 g, 60.3 mmol) and Pd/C (2 g, 20%) in MeOH (50 mL)/THF (100 mL) was stirred at 45° C. overnight under H₂ condition. The filtrates was concentrated in vacuo to afford the desired compound (14 g, 91%) as a brown oil. MS: 230.6 (M+H⁺).

### Step 6: methyl 3-(8-bromonaphthalen-2-yl)propanoate

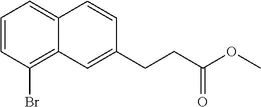 [SMILES: COC(=O)CCc1ccc2cccc(Br)c2c1]

To the mixture of methyl 3-(8-aminonaphthalen-2-yl)propanoate (5 g, 21.81 mmol), copper(I) bromide (9.38 g, 65.4 mmol) and Ts-OH (12.44 g, 65.4 mmol) in ACN (60 mL)/Water (3 mL) was added sodium nitrite (4.51 g, 65.4 mmol) at 0° C. slowly, then the mixture was stirred at RT for 2 hours. The filtrates was concentrated in vacuo to give a residual, which was re-dissolved in EA and the resulting organic mixture was washed with water, saturate NaHCO₃, brine, drine over Na₂SO₄, concentrated in vacuo to give a residual, which was purified by silica gel column and eluted with EA/PE from 0 to 10% to afford the desired compound (3.7 g, 57.9%) as a colorless oil. LCMS: MS: 293.2 (M+H⁺).

### Step 7: methyl 3-(8-bromo-6-(4,4,5,5-tetramethyl-1,3,2-dioxaborolan-2-yl)naphthalen-2-yl)propanoate

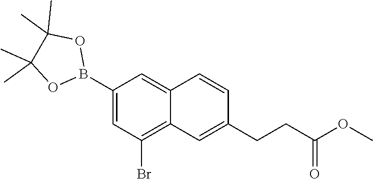

Under Ar, In a round-bottomed flask methyl 3-(8-bromonaphthalen-2-yl)propanoate (1 g, 3.41 mmol), 4,4,4′,4′,5,5,5′,5′-octamethyl-2,2′-bi(1,3,2-dioxaborolane) (2.166 g, 8.53 mmol), dtbpy (0.046 g, 0.171 mmol) and [Ir(OMe)(1,5-cod)]2 (0.056 g, 0.085 mmol) were dissolved in THF (100 mL) under nitrogen to give a color solution. The mixture was stirred at 80° C. for 1 hour. The volatiles was removed under reduced pressure to give a residual, which was purified by silica gel column and eluted with PE to afford the desired compound (1.7 g, 95%) as a colorless oil. MS: 419.2 (M+H⁺).

### Step 8: methyl 3-(8-bromo-6-hydroxynaphthalen-2-yl)propanoate

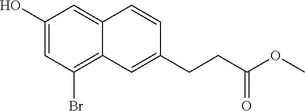 [SMILES: COC(=O)CCc1ccc2cc(O)cc(Br)c2c1]

Under Ar, to the mixture of methyl 3-(8-bromo-6-(4,4,5,5-tetramethyl-1,3,2-dioxaborolan-2-yl)naphthalen-2-yl)propanoate (1.7 g, 3.24 mmol) in acetone (30 mL) was added oxone (1.995 g, 3.24 mmol) in 10 mL of water dropwise. After the addition, the mixture was stirred at RT for 10 mins. Quenched with Na₂SO₃ solution, the volatiles was removed in vacuo to give a residual, which was re-dissolved in EA, and the resulting organic mixture was washed with water, drine over Na₂SO₄, concentrated in vacuo to give a residual, which was purified by silica gel column and eluted with EA/PE from 0 to 30% to afford the desired compound (872 mg, 87%) as a colorless oil. MS: 309.2 (M+H⁺).

### Step 9: 3-(8-bromo-6-hydroxynaphthalen-2-yl)propanoic acid

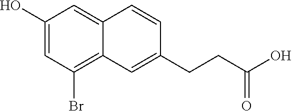 [SMILES: O=C(O)CCc1ccc2cc(O)cc(Br)c2c1]

Under Ar, the mixture of methyl 3-(8-bromo-6-hydroxynaphthalen-2-yl)propanoate (872 mg, 2.82 mmol) and NaOH (338 mg, 8.46 mmol) in THF (5 mL)/Water (5 mL) was stirred at RT for 2 hours. After the removal of the volatiles, the mixture was adjusted to pH=4 with 2N HCl. The precipitates was collected, dried over vacuum to give the desired compound (810 mg, 97%) as a white solid. MS: 295.2 (M+H⁺).

### Step 10: 9-bromo-7-hydroxy-2,3-dihydro-1H-cyclopenta[a]naphthalen-1-one

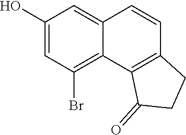 [SMILES: O=C1CCc2ccc3cc(O)cc(Br)c3c21]

Under Ar, the mixture of 3-(8-bromo-6-hydroxynaphthalen-2-yl)propanoic acid (810 mg, 2.74 mmol) in methanesulfonic acid (12 mL) was stirred at 80° C. for 18 hours. Quenched with ice-water, the resulting mixture was extracted with EA for three times. The combined organic layers were washed with brine, dried over Na₂SO₄, and concentrated in vacuo to give a residual, which was purified by silica gel column and eluted with EA/PE from 0 to 50% to afford the desired compound (510 mg, 67.1%) as a gray solid. MS: 277.2 (M+H⁺).

### Step 11: 9-bromo-7-(methoxymethoxy)-2,3-dihydro-1H-cyclopenta[a]naphthalen-1-one

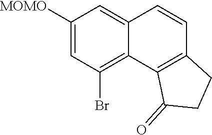

Under Ar, to a solution of 9-bromo-7-hydroxy-2,3-dihydro-1H-cyclopenta[a]naphthalen-1-one (510 mg, 1.840 mmol), TEA (372 mg, 3.68 mmol) in DCM (12 mL) was added bromo(methoxy)methane (345 mg, 2.76 mmol) at 0° C. After stirred at this temperature for 30 mins, water was added. The resulting mixture was extracted with DCM for three times. The combined organic layers were washed with brine, dried over Na₂SO₄, concentrated in vacuo to give a residue, which was purified by silical gel column and eluted with EA/PE from 0 to 30% to afford the desired compound (406 mg, 68.7%) as a white solid. MS: 321.2 (M+H⁺).

### Step 12: 7-(methoxymethoxy)-9-(4,4,5,5-tetramethyl-1,3,2-dioxaborolan-2-yl)-2,3-dihydro-1H-cyclopenta[a]naphthalen-1-one

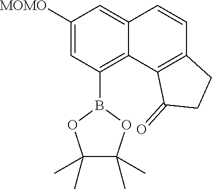

Under Ar, the mixture of 9-bromo-7-(methoxymethoxy)-2,3-dihydro-1H-cyclopenta[a]naphthalen-1-one (406 mg, 1.264 mmol), 4,4,4′,4′,5,5,5′,5′-octamethyl-2,2′-bi(1,3,2-dioxaborolane) (482 mg, 1.896 mmol), potassium acetate (372 mg, 3.79 mmol) and PdCl₂(dppf) (92 mg, 0.126 mmol) in 1,4-Dioxane (3 mL) was stirred at 100° C. under Ar for 10 hours. After the removal off the volatiles, the residue was purified by silica gel column and eluted with EA/Hex from 0 to 30% to afford the desired compound (234 mg, 50.3%) as colorless oil. MS: 369.3 (M+H⁺).

### Intermediate 11: methyl 7-(methoxymethoxy)-9-(4,4,5,5-tetramethyl-1,3,2-dioxaborolan-2-yl)-2,3-dihydro-1H-cyclopenta[a]naphthalene-2-carboxylate

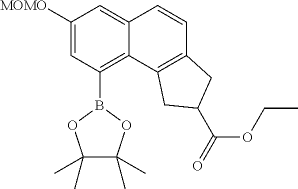

### Step 1: ethyl 9-bromo-7-(methoxymethoxy)-1-oxo-2,3-dihydro-1H-cyclopenta[a]naphthalene-2-carboxylate

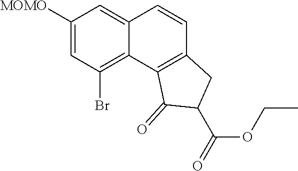

Under Ar, to the mixture of 9-bromo-7-(methoxymethoxy)-2,3-dihydro-1H-cyclopenta[a]naphthalen-1-one (420 mg, 1.308 mmol) in dry THF (10 mL) was added LiHMDS (328 mg, 1.962 mmol) at −78° C., and the mixture was stirred at −78° C. for 1 h, then ethyl carbonocyanidate (143 mg, 1.438 mmol) was added to the above mixture was stirred at −78° C. for another 2 hours. Quenched with water, the resulting mixture was extracted with EA for three times. The combined organic layers were washed with brine, dried over Na₂SO₄, filtered, concentrated in vacuo to give a residual, which was purified by silica gel column and eluted with EA/Hex from 0 to 25% to afford the desired compound (250 mg, 48.6%) as a brown oil. H NMR (DMSO, 400 MHz): 7.92 (d, J=8.4 Hz, 1H), 7.72 (d, J=2.0 Hz, 1H), 7.46 (d, J=8.4 Hz, 1H), 7.44 (d, J=2.0 Hz, 1H), 5.28 (s, 2H), 4.31-4.26 (q, J=7.2 Hz, 2H), 3.91-3.33 (m, 1H), 3.65-3.60 (m, 1H), 3.51 (s, 3H), 3.44-3.38 (m, 1H), 1.33 (t, J=7.2 Hz, 2H).

### Step 2: ethyl 9-bromo-7-hydroxy-2,3-dihydro-1H-cyclopenta[a]naphthalene-2-carboxylate

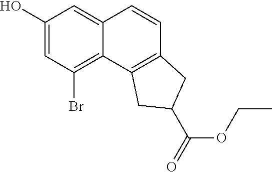 [SMILES: CCOC(=O)C1Cc2ccc3cc(O)cc(Br)c3c2C1]

Under Ar, the mixture of ethyl 9-bromo-7-(methoxymethoxy)-1-oxo-2,3-dihydro-1H-cyclopenta[a]naphthalene-2-carboxylate (250 mg, 0.636 mmol), TES (2 mL), TFA (5 mL) was stirred at room temperature overnight. The volatiles was removed under reduced pressure to give a residual, which was purified by silica gel column and eluted with ethyl acetate/hexane from 0 to 30% to afford the desired compound (103 mg, 48.3%) as a white solid. MS: 335.3 (M+H⁺).

### Step 3: ethyl 9-bromo-7-(methoxymethoxy)-2,3-dihydro-1H-cyclopenta[a]naphthalene-2-carboxylate

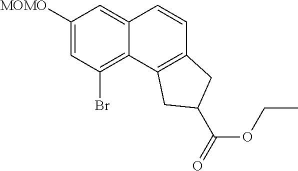

Under Ar, the mixture of ethyl 9-bromo-7-hydroxy-2,3-dihydro-1H-cyclopenta[a]naphthalene-2-carboxylate (103 mg, 0.307 mmol), TEA (62.2 mg, 0.615 mmol) and bromo(methoxy)methane (57.6 mg, 0.461 mmol) in DCM (12 mL) was stirred at ice-water condition for 40 mins. Quenched with water and the resulting mixture was extracted with DCM for three times. The combined organic layers were washed with brine, dried over Na₂SO₄, concentrated in vacuo to give a residual, which was purified by silica gel column and eluted with EA/PE from 0 to 30% afford the desired compound (115 mg, 99%) as a brown oil. MS: 379.3 (M+H⁺).

### Step 4: ethyl 7-(methoxymethoxy)-9-(4,4,5,5-tetramethyl-1,3,2-dioxaborolan-2-yl)-2,3-dihydro-1H-cyclopenta[a]naphthalene-2-carboxylate

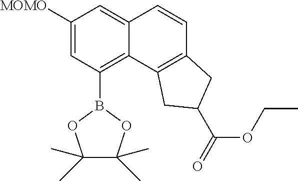

Under Ar, the mixture of ethyl 9-bromo-7-(methoxymethoxy)-2,3-dihydro-1H-cyclopenta[a]naphthalene-2-carboxylate (60 mg, 0.158 mmol), 4,4,4′,4′,5,5,5′,5′-octamethyl-2,2′-bi(1,3,2-dioxaborolane) (60.3 mg), potassium acetate (46.6 mg, 0.475 mmol) and PdCl₂(dppf) (11.58 mg, 0.016 mmol) in 1,4-Dioxane (15 mL) was stirred at 110° C. under for 8 hour. The volatiles was removed under reduced pressure to give a residue, which was purified by silica gel column and eluted with EA/Hex from 0 to 10% to afford the desired compound (32 mg, 47.4%) as colorless oil. MS: 427.5 (M+H⁺).

### Intermediate 12: 7-((tert-butyldimethylsilyl)oxy)-5-(4,4,5,5-tetramethyl-1,3,2-dioxaborolan-2-yl)-2,3-dihydro-1H-pyrrolo[1,2-a]indole

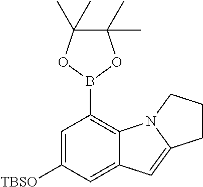 [SMILES: CC1(C)OB(c2cc(O[Si](C)(C)C(C)(C)C)cc3cc4n(c23)CCC4)OC1(C)C]

### Step 1: N-(2,6-dibromo-4-methoxyphenyl)-2,2,2-trifluoroacetamide

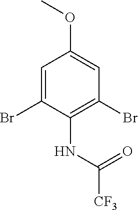 [SMILES: COc1cc(Br)c(NC(=O)C(F)(F)F)c(Br)c1]

Under Ar, to a solution of 2,6-dibromo-4-methoxyaniline (5.0 g, 17.80 mmol) and TEA (3.6 g, 35.6 mmol) in DCM (20 mL) was added 2,6-dibromo-4-methoxyaniline (5.6 g, 26.7 mmol) at 0° C., and then, the mixture was stirred at ice-water condition for 2 h. Diluted with DCM, and the resulting mixture was washed with water, brine, dried over anhydrous Na₂SO₄ and concentrated in vacuo to give a residue, which was purified by silica gel column and eluted with EA/Hex from 0 to 25% to afford the desired compound (4.8 g, 73%) as a yellow solid.

### Step 2: 5-bromo-7-methoxy-2,3-dihydro-1H-pyrrolo[1,2-a]indole

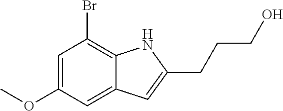 [SMILES: COc1cc(Br)c2[nH]c(CCCO)cc2c1]

Under Ar, the mixture of N-(2,6-dibromo-4-methoxyphenyl)-2,2,2-trifluoroacetamide (4.8 g, 12.73 mmol), pent-4-yn-1-ol (1.07 g, 12.73 mmol), TEA (3.87 g, 38.2 mmol), CuI (0.243 g, 1.27 mmol) and Pd(PPh₃)₂Cl₂ (0.894 g, 1.27 mmol) in dry DMF (20 mL) was stirred at 120° C. for 6 h. Diluted with EA, and the resulting mixture was washed with water, brine, dried over anhydrous Na₂SO₄ and concentrated in vacuo to give a residue, which was purified by silica gel column and eluted with EA/Hex from 0 to 50% to afford the desired compound (1.17 g, 32%) as a white solid. MS: 284.4/286.3 (M+H⁺).

### Step 3: 5-bromo-7-methoxy-2,3-dihydro-1H-pyrrolo[1,2-a]indole

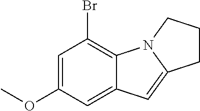

Under Ar, to a solution of 3-(7-bromo-5-methoxy-1H-indol-2-yl)propan-1-ol (1170 mg, 4.12 mmol) and PPh₃ (1620 mg, 6.18 mmol) in dry THF (40 mL) were added DIAD (1250 mg, 6.18 mmol) at ice-water condition. After stirred at 25° C. for 16 h. Diluted with EA, and the resulting mixture was washed with water, brine, dried over anhydrous Na₂SO₄ and concentrated in vacuo to give a residue, which was purified by silica gel column and eluted with EA/Hex from 0 to 10% to afford the desired compound (930 mg, 85%) as a yellow solid. MS: 266.4/268.3 (M+H⁺)

### Step 4: 5-bromo-2,3-dihydro-1H-pyrrolo[1,2-a]indol-7-ol

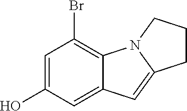 [SMILES: Oc1cc(Br)c2c(c1)cc1n2CCC1]

Under Ar, the mixture of 5-bromo-7-methoxy-2,3-dihydro-1H-pyrrolo[1,2-a]indole (210 mg, 0.789 mmol) and tribromo borane (990 mg, 3.95 mmol) in dry DCM (15 mL) was stirred at 0° C. for 1 h. Quenched with NH₄Cl aq. And the resulting mixture was extracted with EA for 3 times. The combined organic layers were washed with brine, dried over anhydrous Na₂SO₄ and concentrated in vacuo to give a residue, which was purified by silica gel column and eluted with EA/Hex from 0 to 20% to afford the desired compound (196 mg, 99%) as yellow oil. MS: 252.4/254.2 (M+H⁺)

### Step 5: 5-bromo-7-((tert-butyldimethylsilyl)oxy)-2,3-dihydro-1H-pyrrolo[1,2-a]indole

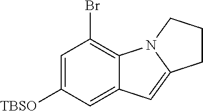 [SMILES: CC(C)(C)[Si](C)(C)Oc1cc(Br)c2c(c1)cc1n2CCC1]

Under Ar, to a mixture of 5-bromo-2,3-dihydro-1H-pyrrolo[1,2-a]indol-7-ol (196 mg, 0.78 mmol) and TEA (157 mg, 1.55 mmol) in DCM (10 mL) was added TBSCl (140 mg, 0.93 mmol) at 0° C. After stirred at RT for one hour, water was added and the resulting mixture was extracted with DCM for 3 times. The combined organic layers were washed with brine, dried over anhydrous Na₂SO₄ and concentrated in vacuo to give a residue, which was purified by silical gel column and eluted with EA/Hex from 0 to 5% to afford the desired compound (276 mg, 97%) as a colorless oil. MS: 366.1/368.1 (M+H⁺)

### Step 6: 7-((tert-butyldimethylsilyl)oxy)-5-(4,4,5,5-tetramethyl-1,3,2-dioxaborolan-2-yl)-2,3-dihydro-1H-pyrrolo[1,2-a]indole

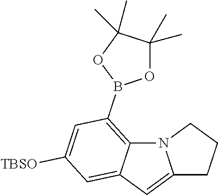 [SMILES: CC1(C)OB(c2cc(O[Si](C)(C)C(C)(C)C)cc3cc4n(c23)CCC4)OC1(C)C]

Under Ar, the mixture of 5-bromo-7-((tert-butyldimethylsilyl)oxy)-2,3-dihydro-1H-pyrrolo[1,2-a]indole (276 mg, 0.75 mmol), 4,4,4′,4′,5,5,5′,5′-octamethyl-2,2′-bi(1,3,2-dioxaborolane) (383 mg, 1.51 mmol), KOAc (222 mg, 2.26 mmol) and Pd(dppf)Cl₂ (55 mg, 0.07 mmol) in dioxane (4 mL) was stirred at 90° C. for 4 hours. The volatiles was removed under reduced pressure to give a residual, which was purified by silica gel column and eluted with ethyl acetate/hexane from 0 to 30% to afford the desired compound (230 mg, 74%) as colorless oil. MS: 414.3 (M+H⁺).

### Intermediate 13: 2-(7-(benzyloxy)-1,3-dihydronaphtho[1,2-c]furan-9-yl)-4,4,5,5-tetramethyl-1,3,2-dioxaborolane

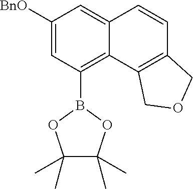 [SMILES: CC1(C)OB(c2cc(OCc3ccccc3)cc3ccc4c(c23)COC4)OC1(C)C]

### Step 1: ((5-(benzyloxy)-3-bromo-2-(2,5-dihydrofuran-3-yl)phenyl)ethynyl)trimethylsilane

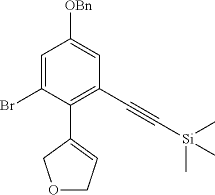 [SMILES: C[Si](C)(C)C#Cc1cc(OCc2ccccc2)cc(Br)c1C1=CCOC1]

Under Ar, the mixture of ((5-(benzyloxy)-3-bromo-2-iodophenyl)ethynyl)trimethylsilane (2.48 g, 5.10 mmol), PdCl₂(dppf) (373 mg, 0.51 mmol), Na₂CO₃ (1.62 g, 15.3 mmol) and 2-(2,5-dihydrofuran-3-yl)-4,4,5,5-tetramethyl-1,3,2-dioxaborolane (1000 mg, 5.10 mmol) in Dioxane (24 mL) and water (6 mL) was stirred at 100° C. for 3 h. After cooling down to RT, diluted with water, and the resulting mixture was extracted with EA for 3 times. The combined organic layers were washed with brine, dried over anhydrous Na₂SO₄ and concentrated in vacuo to give a residue, which was purified by silica gel column and eluted with MTBE/PE from 0 to 30% to afford the title compound (1.05 g, 48.2%) as yellow oil. MS: 427.2 (M+H⁺).

### Step 2: 3-(4-(benzyloxy)-2-bromo-6-ethynylphenyl)-2,5-dihydrofuran

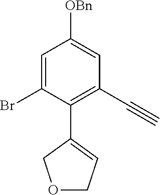 [SMILES: C#Cc1cc(OCc2ccccc2)cc(Br)c1C1=CCOC1]

Under Ar, the mixture of ((5-(benzyloxy)-3-bromo-2-(2,5-dihydrofuran-3-yl)phenyl)ethynyl)trimethylsilane (1.05 g, 2.46 mmol) and K₂CO₃ (1.70 g, 12.28 mmol) in MeOH (20 mL) was stirred at RT for 1 h. The filtrates was concentrated in vacuo to give a residue, which was purified by silica gel column and eluted with MTBE/Hex from 0 to 2% to afford the title compound (720 mg, 83%) as yellow oil.

MS:355.4 (M+H⁺).

### Step 3: 7-(benzyloxy)-9-bromo-1,3-dihydronaphtho[1,2-c]furan

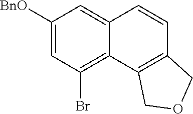 [SMILES: Brc1cc(OCc2ccccc2)cc2ccc3c(c12)COC3]

Under Ar, the mixture of 3-(4-(benzyloxy)-2-bromo-6-ethynylphenyl)-2,5-dihydrofuran (720 mg, 2.03 mmol) and PtCl₂ (162 mg, 0.61 mmol) in Toluene (20 mL) was stirred at refluxing for 8 h. The volatiles was removed under reduced pressure to give a residue, which was purified by silica gel column and eluted with MTBE/Hex from 0 to 3% to afford the title compound (150 mg, 20.8%) as a yellow solid. MS: 355.4 (M+H⁺).

### Step 4: 2-(7-(benzyloxy)-1,3-dihydronaphtho[1,2-c]furan-9-yl)-4,4,5,5-tetramethyl-1,3,2-dioxaborolane

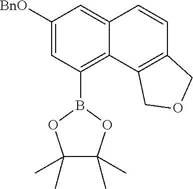 [SMILES: CC1(C)OB(c2cc(OCc3ccccc3)cc3ccc4c(c23)COC4)OC1(C)C]

Under Ar, the mixture of 7-(benzyloxy)-9-bromo-1,3-dihydronaphtho[1,2-c]furan (60 mg, 0.169 mmol), 4,4,4′,4′,5,5,5′,5′-octamethyl-2,2′-bi(1,3,2-dioxaborolane) (64 mg, 0.253 mmol), potassium acetate (50 mg, 0.508 mmol) and PdCl₂(dppf) (12.4 mg, 0.018 mmol) in Dioxane (5 mL) was stirred at 100° C. for 2 h. The volatiles was removed under reduced pressure to give a residue, which was purified by silica gel column and eluted with EA/Hex from 0 to 5% to afford the desired compound (50 mg, 73.6%) as colorless oil. MS: 403.3 (M+H⁺).

### Intermediate 14: 8-(methoxymethoxy)-10-(4,4,5,5-tetramethyl-1,3,2-dioxaborolan-2-yl)pyrrolo[2,1-a]isoquinoline

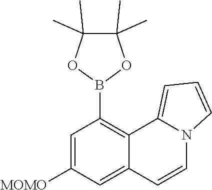

### Step 1: tert-butyl 2-(2-bromo-4-methoxyphenyl)-1H-pyrrole-1-carboxylate

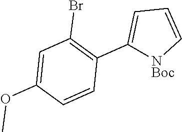 [SMILES: COc1ccc(-c2cccn2C(=O)OC(C)(C)C)c(Br)c1]

Under Ar, the mixture of 2-bromo-1-iodo-4-methoxybenzene (500 mg, 1.598 mmol), (1-(tert-butoxycarbonyl)-1H-pyrrol-2-yl)boronic acid (371 mg, 1.758 mmol), K₂CO₃ (662 mg, 4.79 mmol) and PdCl₂(PPh₃)₂ (112 mg, 0.12 mmol) in dioxane (10 mL) and water (2.5 mL) was stirred at 100° C. for 3 h. After cooling down to RT, Water was added, and the resulting mixture was extracted with EA for 3 times. The combined organic layers were washed with brine, dried over anhydrous Na₂SO₄ and concentrated in vacuo to give a residue, which was purified by silica gel column and eluted with MTBE/Hex from 0 to 5% to afford the desired compound (470 mg, 84%) as a colorless oil. MS: 352.3 (M+H⁺).

### Step 2: 2-(2-bromo-4-methoxyphenyl)-1H-pyrrole

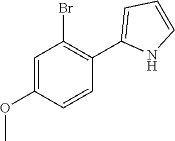

Under Ar, the mixture of tert-butyl 2-(2-bromo-4-methoxyphenyl)-1H-pyrrole-1-carboxylate (470 mg, 1.334 mmol) and MeONa (360 mg, 6.67 mmol) in MeOH (5 mL) and THF (5 mL) was stirred at RT for 3 hour. Water was added and the resulting mixture was extracted with EA for 3 times. The combined EA layers were washed with brine, dried over anhydrous Na₂SO₄ and concentrated in vacuo to give a residue, which was purified by silica gel column and eluted with MTBE/Hex from 0 to 5% to afford the desired compound (270 mg, 80%) as colorless oil. MS: 252.3 (M+H⁺).

### Step 3: 2-(2-bromo-4-methoxyphenyl)-1-(2,2-dimethoxyethyl)-1H-pyrrole

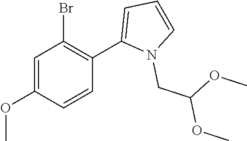 [SMILES: COc1ccc(-c2cccn2CC(OC)OC)c(Br)c1]

Under Ar, the mixture of 2-bromo-1,1-dimethoxyethane (905 mg, 5.35 mmol), 2-(2-bromo-4-methoxyphenyl)-1H-pyrrole (270 mg, 1.071 mmol) and Cs₂CO₃ (1745 mg, 5.35 mmol) in DMF (5 mL) was stirred at 110° C. for 30 h. After cooling down to RT, water was added and the resulting mixture was extracted with EA for 3 times. The combined EA layers were washed with brine, dried over anhydrous Na₂SO₄ and concentrated in vacuo to give a residual, which was purified by silica gel column and eluted with MTBE/Hex from 0 to 5% to afford the desired compound (160 mg, 80%) as colorless oil. MS: 340.3 (M+H⁺).

### Step 4: 10-bromo-8-methoxypyrrolo[2,1-a]isoquinoline

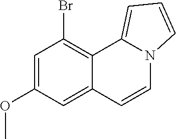 [SMILES: COc1cc(Br)c2c(ccn3cccc23)c1]

Under Ar, the mixture of 2-(2-bromo-4-methoxyphenyl)-1-(2,2-dimethoxyethyl)-1H-pyrrole (160 mg, 0.47 mmol) and TfOH (706 mg, 4.7 mmol) in DCM (5 mL) was stirred at RT for 16 h. Water was added and the resulting mixture was extracted with EA for 3 times. The combined EA layers were washed with brine, dried over anhydrous Na₂SO₄ and concentrated in vacuo to give a residual, which was purified by silica gel column and eluted with MTBE/Hex from 0 to 5% to afford the desired compound (160 mg, 54%) as a white solid. MS: 276.3 (M+H⁺).

### Step 5: 10-bromopyrrolo[2,1-a]isoquinolin-8-ol

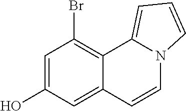 [SMILES: Oc1cc(Br)c2c(ccn3cccc23)c1]

Under Ar, to a solution of 10-bromo-8-methoxypyrrolo[2,1-a]isoquinoline (70 mg, 0.254 mmol) in 1,2-Dichloroethane (5 mL) was added BBr₃ (635 mg, 2.54 mmol) at RT, and then, the mixture was stirred at 70° C. for 16 h. After cooling down to RT, the mixture was added to NaHCO₃ aq at ice-water condition. The resulting mixture was extracted with DCM for 3 times. The combined organic layers were washed with brine, dried over anhydrous Na₂SO₄ and concentrated in vacuo to afford the desired compound (60 mg, crude) as brown oil. MS: 262.3 (M+H⁺).

### Step 6: 10-bromo-8-(methoxymethoxy)pyrrolo[2,1-a]isoquinoline

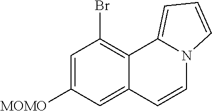

Under Ar, to a solution of 10-bromopyrrolo[2,1-a]isoquinolin-8-ol (60 mg, crude) and DIEA (29 mg, 0.458 mmol) in DCM (5 mL) was added MOMBr (43 mg, 0.343 mmol) at 0° C. Then, the reaction mixture was stirred at 0° C. for 1 h. quenched with NaHCO₃ aq, and the resulting mixture was extracted with DCM for 3 times. The combined organic layers were washed with brined, dried over anhydrous Na₂SO₄ and concentrated in vacuo to give a residue, which was purified by silica gel column and eluted with MTBE/Hex from 0 to 5% to afford the desired compound (50 mg) as a white solid. MS: 306.3 (M+H⁺).

### Step 7: 8-(methoxymethoxy)-10-(4,4,5,5-tetramethyl-1,3,2-dioxaborolan-2-yl)pyrrolo[2,1-a]isoquinoline

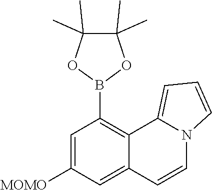

Under Ar, the mixture of 9-bromo-7-(methoxymethoxy)-1,3-dihydronaphtho[1,2-c]furan (50 mg, 0.163 mmol), 4,4,4′,4′,5,5,5′,5′-octamethyl-2,2′-bi(1,3,2-dioxaborolane) (62 mg, 0.245 mmol), potassium acetate (48 mg, 0.49 mmol) and PdCl₂(dppf) (12 mg, 0.016 mmol) in Dioxane (5 mL) was stirred at 100° C. for 2 h. The volatiles was removed under reduced pressure to give a residual, which was purified by silica gel column and eluted with MTBE/Hex from 0 to 5% to afford the desired compound (50 mg, 87%) as a white solid. MS: 354.4 (M+H⁺).

### Intermediate 15: tert-butyl (1R,5S)-3-(2,8-difluoro-7-(7-(methoxymethoxy)-2,3-dihydro-1H-cyclopenta[a]naphthalen-9-yl)quinazolin-4-yl)-3,8-diazabicyclo[3.2.1]octane-8-carboxylate

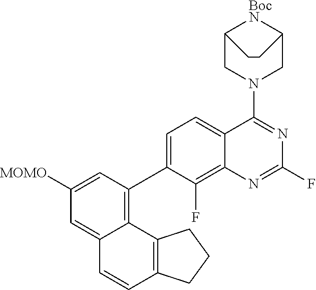

### Step 1: 7-bromo-8-fluoroquinazoline-2,4(1H,3H)-dione

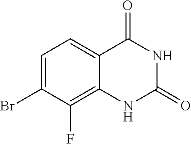 [SMILES: O=c1[nH]c(=O)c2ccc(Br)c(F)c2[nH]1]

Under Ar, 2-amino-4-bromo-3-fluorobenzoic acid (3 g, 12.8 mol) and urea (46 g, 768 mol) were added to a 250 mL round bottom flask. The reaction mixture was stirred at 200° C. for 3 h. After the reaction completed, water was added to the reaction mixture and stirred the reaction mixture at 100° C. for 1 h. Then allowed the reaction mixture to reach room temperature, the solid was filtered and dried to afford the title compound (3.5 g) without any further purification. MS: 259.1 (M+H⁺).

### Step 2: 7-bromo-2,4-dichloro-8-fluoroquinazoline

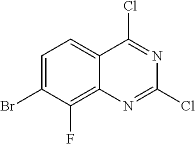 [SMILES: Fc1c(Br)ccc2c(Cl)nc(Cl)nc12]

Under Ar, 7-bromo-8-fluoroquinazoline-2,4(1H,3H)-dione (3.5 g, 13.6 mmol) was suspended in 72 mL of phosphoryl chloride at room temperature, then 24 mL of DIPEA (135 mmol) was added. Subsequently, the reaction mixture was heated at 110° C. for 10 h. After the reaction completed, excessive phosphoryl chloride was removed by vacuum pump and ice water were added to the residue. The resulting mixture was extracted with EA for 3 times, and the combined organic layers were washed with brine, dried over anhydrous Na₂SO₄ and concentrated in vacuo to give a residual, which was purified by silica gel column and eluted with PE/EA (5:1) to afford the title compound (1.2 g, 30%) as yellow solid. MS: 295.1 (M+H⁺).

### Step 3: tert-butyl (1R,5S)-3-(7-bromo-2-chloro-8-fluoroquinazolin-4-yl)-3,8-diazabicyclo[3.2.1]octane-8-carboxylate

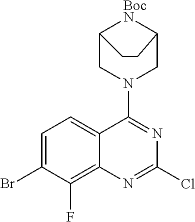 [SMILES: CC(C)(C)OC(=O)N1C2CCC1CN(c1nc(Cl)nc3c(F)c(Br)ccc13)C2]

Under Ar, 7-bromo-2,4-dichloro-8-fluoroquinazoline (1.2 g, 4.0 mmol) and tert-butyl 3,8-diazabicyclo[3.2.1]octane-8-carboxylate (850 mg, 4.0 mmol) were dissolved in 10 mL DCM followed with addition of 2.1 mL of DIPEA (12 mmol). The reaction mixture was stirred at room temperature for 3 h. After the reaction completed, saturated ammonium chloride solution was added and the resulting mixture was extracted with DCM for 3 times. The combined organic layers were washed with brine, dried over anhydrous Na₂SO₄ and concentrated in vacuo to give a residual, which was purified by silica gel column and eluted with PE/EA (5:1) to afford the title compound (1.4 g, 73%) as yellow solid. MS: 471.3 (M+H⁺). ¹H NMR (400 MHz, CDCl₃) δ 7.51-7.45 (m, 2H), 4.36 (brs, 4H), 3.63 (brs, 2H), 1.99-1.89 (m, 2H), 1.73 (m, 2H), 1.51 (s, 9H).

### Step 4: tert-butyl (1R,5S)-3-(7-bromo-2,8-difluoroquinazolin-4-yl)-3,8-diazabicyclo[3.2.1]octane-8-carboxylate

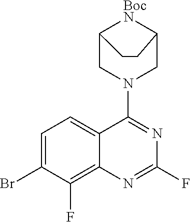 [SMILES: CC(C)(C)OC(=O)N1C2CCC1CN(c1nc(F)nc3c(F)c(Br)ccc13)C2]

Under Ar, tert-butyl (1R,5S)-3-(7-bromo-2-chloro-8-fluoroquinazolin-4-yl)-3,8-diazabicyclo[3.2.1]octane-8-carboxylate (1.2 g, 2.55 mmol), KF (3.0 g, 51 mmol) and 10 mL DMSO were added to a 100 mL sealed tube. The reaction mixture was stirred at 120° C. for 4 h. After the reaction completed, water was added to the mixture. The resulting mixture was extracted with EA for 3 times, and the combined organic layers were washed with brine, dried over anhydrous Na₂SO₄ and concentrated in vacuo to give a residual, which was purified by silica gel column and eluted with PE/EA (5:1) to afford the title compound (686 mg, 60%) as yellow solid. LC-MS (m/z): MS: 455.3 (M+H⁺). ¹H NMR (400 MHz, CDCl₃) δ 7.69-7.38 (m, 2H), 4.39 (m, 4H), 3.64 (brs, 2H), 1.95 (m, 2H), 1.72 (m, 2H), 1.51 (s, 9H).

### Step 5: tert-butyl (1R,5S)-3-(2,8-difluoro-7-(7-(methoxymethoxy)-2,3-dihydro-1H-cyclopenta[a]naphthalen-9-yl)quinazolin-4-yl)-3,8-diazabicyclo[3.2.1]octane-8-carboxylate

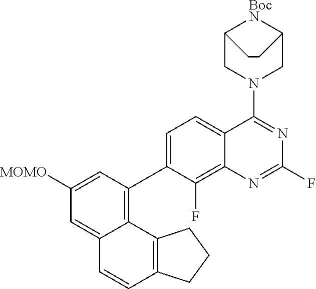

Under Ar, the mixture of tert-butyl (1R,5S)-3-(7-bromo-2,8-difluoroquinazolin-4-yl)-3,8-diazabicyclo[3.2.1]octane-8-carboxylate (686 mg, 1.51 mmol), 2-(7-(methoxymethoxy)-2,3-dihydro-1H-cyclopenta[a]naphthalen-9-yl)-4,4,5,5-tetramethyl-1,3,2-dioxaborolane (640 mg, 1.81 mmol), K₃PO₄ (960 mg, 4.53 mmol) and Pd(dppf)Cl₂·CH₂Cl₂ (122 mg, 0.151 mmol) in THF (50 mL) and Water (10 mL) was stirred at 65° C. for 4 hours. After cooling down to room temperature, water was added, the resulting mixture was extracted with EA for 3 times. The combined organic layers were washed with brine, dried over anhydrous Na₂SO₄ and concentrated in vacuo to give a residual, which was purified by silica gel column and eluted with PE/EA (5:1) to afford the title compound (537 mg, 58%) as yellow solid. MS: 603.5 (M+H⁺).

### Intermediate 16: tert-butyl (1R,5S)-3-(2,6,8-trifluoro-7-(7-(methoxymethoxy)-2,3-dihydro-1H-cyclopenta[a]naphthalen-9-yl)quinazolin-4-yl)-3,8-diazabicyclo[3.2.1]octane-8-carboxylate

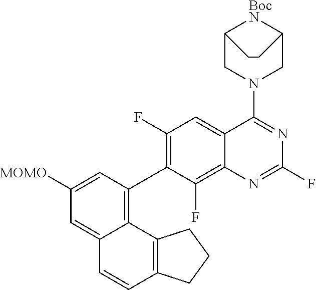

### Step 1: 3-bromo-2,4-difluoro-6-iodoaniline

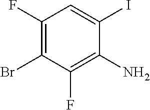 [SMILES: Nc1c(I)cc(F)c(Br)c1F]

3-bromo-2,4-difluoroaniline (756 mg, 3.6 mmol), iodine (1 g, 4 mmol) and silver sulfate (1.12 g, 3.6 mmol) were added to ethanol (10 mL) and the reaction mixture was stirred at room temperature for 6 hours. After the reaction completed, filtered the reaction mixture and the filtrate concentrated in vacuo to give a residual, which was purified by silica gel column and eluted with PE to afford the title compound (1.1 g, 90% yield) as a brown solid.

### Step 2: methyl 2-amino-4-bromo-3,5-difluorobenzoate

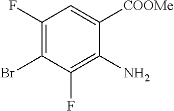 [SMILES: COC(=O)c1cc(F)c(Br)c(F)c1N]

Under carbon monoxide, a solution of 3-bromo-2,4-difluoro-6-iodoaniline (1 g, 3 mmol) and Pd(dppf)Cl₂·CH₂Cl₂ (121 mg, 0.15 mmol) in methanol (40 mL) was stirred at room temperature for 5 minutes. Then triethylamine (21 mmol) was added and stirred at 40° C. overnight. After the reaction completed, the solvent was concentrated and the residue was purified by flash chromatography on silica gel eluting with PE/EA (20/1) to afford the title compound (558 mg, 70% yield) as yellow solid. ¹H NMR (400 MHz, CDCl₃) δ 7.45 (dd, J=9.2, 2.2 Hz, 1H), 5.72 (brs, 2H), 3.89 (s, 3H).

### Step 3: 7-bromo-6,8-difluoroquinazoline-2,4(1H,3H)-dione

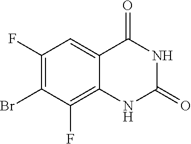 [SMILES: O=c1[nH]c(=O)c2cc(F)c(Br)c(F)c2[nH]1]

Methyl 2-amino-4-bromo-3,5-difluorobenzoate (220 mg, 0.8 mmol) was dissolved in DCM (5 mL), then chlorosulfonyl isocyanate (129 mg, 0.9 mmol) was added at 0° C. Subsequently, the reaction mixture was stirred at room temperature for 1 h until the starting material disappeared. The reaction mixture was concentrated in vacuo, and 20 mL water was added to the mixture which was stirred at 80° C. for 1 h. Then reaction mixture was recovered to room temperature and 1 mL 10M NaOH was added to the mixture and stirred at 80° C. for 1 h. Then the reaction mixture was recovered to room temperature and adjusted to PH=1 with concentrated hydrochloric acid. The solids were collected after filtration and washed with water to afford the title compound (91 mg, 41% yield) as yellow solid.

### Step 4: tert-butyl (1R,5S)-3-(2,6,8-trifluoro-7-(7-(methoxymethoxy)-2,3-dihydro-1H-cyclopenta[a]naphthalen-9-yl)quinazolin-4-yl)-3,8-diazabicyclo[3.2.1]octane-8-carboxylate

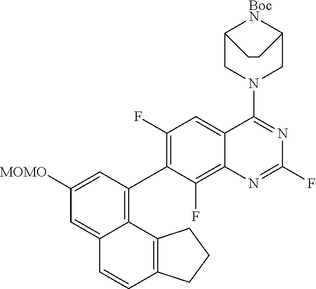

The subsequently procedure for preparing the title compound was the same as Intermediate 15 to give tert-butyl (1R,5S)-3-(2,6,8-trifluoro-7-(7-(methoxymethoxy)-2,3-dihydro-1H-cyclopenta[a]naphthalen-9-yl)quinazolin-4-yl)-3,8-diazabicyclo[3.2.1]octane-8-carboxylate as yellow solid. MS: 621.4 (M+H⁺).

### Example 1

### 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)naphtho[1,2-b]furan-7-ol

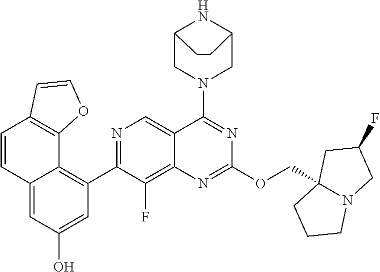 [SMILES: Oc1cc(-c2ncc3c(N4CC5CCC(C4)N5)nc(OC[C@@]45CCCN4C[C@H](F)C5)nc3c2F)c2c(ccc3ccoc32)c1]

### Step 1: tert-butyl (1R,5S)-3-(2,7-dichloro-8-fluoropyrido[4,3-d]pyrimidin-4-yl)-3,8-diazabicyclo[3.2.1]octane-8-carboxylate

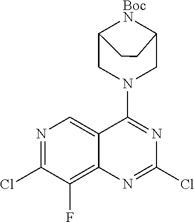 [SMILES: CC(C)(C)OC(=O)N1C2CCC1CN(c1nc(Cl)nc3c(F)c(Cl)ncc13)C2]

Under Ar, to a solution of 2,4,7-trichloro-8-fluoropyrido[4,3-d]pyrimidine (505 mg, 2.000 mmol) and tert-butyl (1R,5S)-3,8-diazabicyclo[3.2.1]octane-8-carboxylate (425 mg, 2.000 mmol) in THF (15 mL) was added DIPEA (646 mg, 5.00 mmol) at −20° C., then the reaction was stirred at room temperature for 0.5 hour. Water was added, the resulting mixture was extracted with ethyl acetate three times. The combined organic layers were rinsed with brine, dried over sodium sulfate, filtered, and concentrated in vacuo to give a residue, which was purified by silica gel column and eluted with ethyl acetate/hexane from 0 to 30% to afford the title compound (810 mg, 95%) as an oil. MS:428.3 (M+H⁺).

### Step 2: tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate

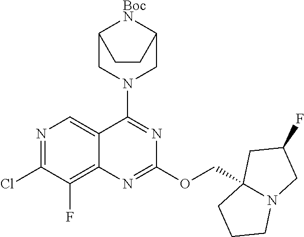 [SMILES: CC(C)(C)OC(=O)N1C2CCC1CN(c1nc(OC[C@@]34CCCN3C[C@H](F)C4)nc3c(F)c(Cl)ncc13)C2]

Under Ar, to the solution of ((2R,7aS)-2-fluorotetrahydro-1H-pyrrolizin-7a(5H)-yl)methanol (362 mg, 2.27 mmol) in tetrahydrofuran (10.0 mL) was added sodium hydride (756 mg, 18.9 mmol) at rt, then the mixture was stirred at 0.5 h at this temperature. A solution of tert-butyl (1R,5S)-3-(2,7-dichloro-8-fluoropyrido[4,3-d]pyrimidin-4-yl)-3,8-diazabicyclo[3.2.1]octane-8-carboxylate (810 mg, 1.89 mmol) in tetrahydrofuran (10.0 mL) was added to the above mixture at ice-water condition. After stirred at rt for 2 hours, aqueous NH₄Cl solution was added. The resulting mixture was extracted EA for 3 times, the combined organic layers were washed with brine, dried over sodium sulfate, filtered, and concentrated in vacuo to give a residual, which was purified by silica gel column and eluted with methanol/dichloromethane from 0 to 10% to afford the title compound (812 mg, 78%) as light yellow solid. MS: 551.3 (M+H⁺).

### Step 3: tert-butyl (1R,5S)-3-(8-fluoro-2-(((2R,7aS)-2-fluorotetrahydro-1H-pyrrolizin-7a(5H)-yl)methoxy)-7-(7-(methoxymethoxy)naphtho[1,2-b]furan-9-yl)pyrido[4,3-d]pyrimidin-4-yl)-3,8-diazabicyclo[3.2.1]octane-8-carboxylate

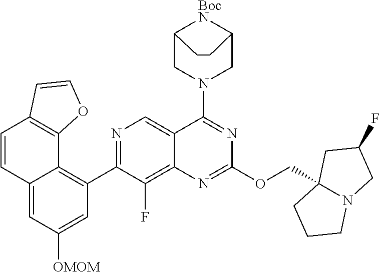

Under Ar, the mixture of tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (54 mg, 0.098 mmol), 2-(7-(methoxymethoxy) naphtho[1,2-b]furan-9-yl)-4,4,5,5-tetramethyl-1,3,2-dioxa borolane (30 mg, 0.085 mmol), Ruphos-Pd-G3 (16 mg, 0.02 mmol) and K₃PO₄ (62 mg, 0.293 mmol) in THF/water(4 mL/2 mL) was stirred at 60° C. under for 2 hours. After cooling down to rt, water was added, the resulting mixture was extracted with EA for 3 times. The combined EA layers were washed with brine, dried over anhydrous Na₂SO₄ and concentrated in vacuo to give a residual, which was purified by silica gel column and eluted with DCM/MeOH=20:1 to afford the title compound (30 mg, 41.3%) as yellow oil. MS: 743.6 (M+H⁺)

### Step 4: 9-(4-((1R,5S)-3,8-diazabicyclo[3.2.1]octan-3-yl)-8-fluoro-2-(((2R, 7aS)-2-fluorotetrahydro-1H-pyrrolizin-7a(5H)-yl)methoxy)pyrido[4,3-d]pyrimidin-7-yl)naphtho[1,2-b]furan-7-ol

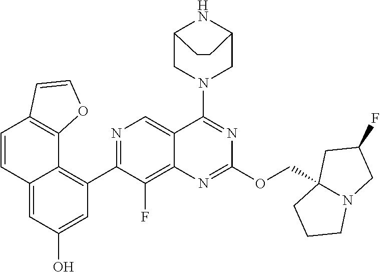 [SMILES: Oc1cc(-c2ncc3c(N4CC5CCC(C4)N5)nc(OC[C@@]45CCCN4C[C@H](F)C5)nc3c2F)c2c(ccc3ccoc32)c1]

Under Ar, to a solution of tert-butyl (1R,5S)-3-(8-fluoro-2-(((2R,7aS)-2-fluoro tetrahydro-1H-pyrrolizin-7a(5H)-yl)methoxy)-7-(7-(methoxymethoxy)naphtho[1,2-b]furan-9-yl)pyrido[4,3-d]pyrimidin-4-yl)-3,8-diazabicyclo[3.2.1]octane-8-carboxylate (30 mg, 0.04 mmol) in MeCN (2 mL) was added 4M HCl/dioxane (1 mL) at ice-water condition. After stirred at rt for 1 hour, quenched with NaHCO₃ aq, extracted with DCM for 3 times. The combined DCM layers were dried over anhydrous Na₂SO₄ and concentrated in vacuo to give a residual, which was purified by pre-HPLC to the title compound (5 mg, 20.7%) as a white solid.

¹H NMR (400 MHz, DMSO) δ 9.97 (s, 1H), 9.15 (s, 1H), 7.69 (m, 2H), 7.63 (d, J=2.0 Hz, 1H), 7.42 (d, J=2.0 Hz, 1H), 7.17 (d, J=2.0 Hz, 1H), 6.93 (d, J=2.0 Hz, 1H), 5.40-5.20 (m, 1H), 4.60-4.45 (m, 2H), 4.20-4.06 (m, 2H), 3.70-2.80 (m, 8H), 2.05-1.67 (m, 10H). MS: 599.6 (M+H⁺).

### Example 2

### 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-2,3-dihydro-1H-cyclopenta[a]naphthalen-7-ol

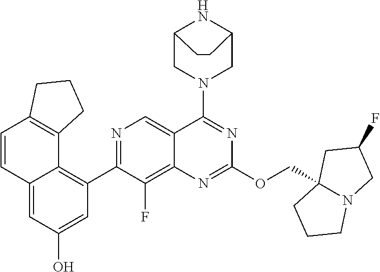 [SMILES: Oc1cc(-c2ncc3c(N4CC5CCC(C4)N5)nc(OC[C@@]45CCCN4C[C@H](F)C5)nc3c2F)c2c3c(ccc2c1)CCC3]

### Step 1: tert-butyl (1R,5S)-3-(8-fluoro-2-(((2R,7aS)-2-fluorotetrahydro-1H-pyrrolizin-7a(5H)-yl)methoxy)-7-(7-(methoxymethoxy)-2,3-dihydro-1H-cyclopenta[a]naphthalen-9-yl)pyrido[4,3-d]pyrimidin-4-yl)-3,8-diazabicyclo[3.2.1]octane-8-carboxylate

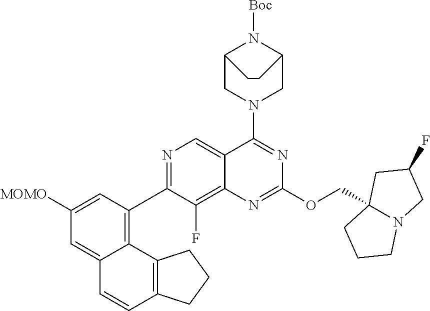

Under Ar, the mixture of tert-butyl (1R,5S)-3-(7-chloro-8-fluoro-2-(((2R, 7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (62.2 mg, 0.113 mmol), 2-(7-(methoxymethoxy)-2,3-dihydro-1H-cyclopenta[a]naphthalen-9-yl)-4,4,5,5-tetramethyl-1,3,2-dioxaborolane (40 mg, 0.113 mmol), Ruphos-Pd-G3 (14.18 mg, 0.017 mmol) and K₃PO₄ (120 mg, 0.565 mmol) in THF (5 mL)/Water (3 mL) was stirred at 60° C. for 2 hours. After cooling down to rt, the mixture was extracted with EA for 3 times. The combined organic layers were washed with brine, concentrated in vacuo to give a residual, which was purified by silica gel column and eluted with DCM/MeOH=19:1 to afford the title compound (71 mg, 85%) as a colorless oil. MS: 743.9 (M+H⁺).

### Step 2: 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-2,3-dihydro-1H-cyclopenta[a]naphthalen-7-ol

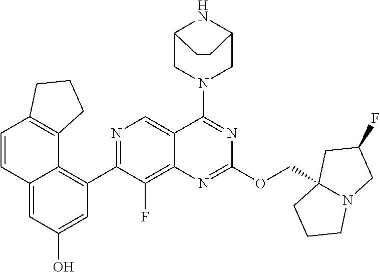 [SMILES: Oc1cc(-c2ncc3c(N4CC5CCC(C4)N5)nc(OC[C@@]45CCCN4C[C@H](F)C5)nc3c2F)c2c3c(ccc2c1)CCC3]

Under Ar, to a solution of tert-butyl (1R,5S)-3-(8-fluoro-2-(((2R,7aS)-2-fluoro tetrahydro-1H-pyrrolizin-7a(5H)-yl)methoxy)-7-(7-(methoxymethoxy)-2,3-dihydro-1H-cyclopenta[a]naphthalen-9-yl)pyrido[4,3-d]pyrimidin-4-yl)-3,8-diazabicyclo[3.2.1]octane-8-carboxylate (71 mg, 0.096 mmol) in Acetonitrile (10 mL) was added HCl/dioxane(10 mL) at ice-water condition. After the mixture was stirred at rt for 2 hours, quenched with NaHCO₃ aq, extracted with DCM for 3 times. The combined DCM layers were dried over anhydrous Na₂SO₄ and concentrated in vacuo to give a residual, which was purified by pre-HPLC to the title compound (21 mg, 36.7%) as a white solid. ¹H NMR (400 MHz, DMSO-d₆) δ 9.09 (s, 1H), 7.62 (d, J=8.0 Hz, 1H), 7.36 (d, J=8.0 Hz, 1H), 7.28 (d, J=1.6 Hz, 1H), 6.99 (d, J=1.6 Hz, 1H), 5.34-5.21 (m, 1H), 4.52-4.36 (m, 2H), 4.14-4.01 (m, 2H), 3.68-3.01 (m, 8H), 2.88-2.82 (m, 3H), 2.20-2.00 (m, 5H), 1.85-1.65 (m, 9H). MS: 599.8 (M+H⁺).

### Example 3

### 4-((1R,5S)-3,8-diazabicyclo[3.2.1]octan-3-yl)-7-(1,2-dihydronaphtho[2,1-b]furan-9-yl)-8-fluoro-2-(((2R,7aS)-2-fluorotetrahydro-1H-pyrrolizin-7a(5H)-yl)methoxy)pyrido[4,3-d]pyrimidine

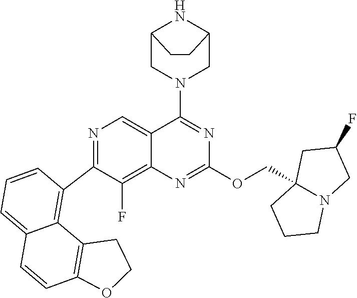 [SMILES: Fc1c(-c2cccc3ccc4c(c23)CCO4)ncc2c(N3CC4CCC(C3)N4)nc(OC[C@@]34CCCN3C[C@H](F)C4)nc12]

Example 3 was prepared essentially the same protocol described in EXAMPLE 1

With 2-(1,2-dihydronaphtho[2,1-b]furan-9-yl)-4,4,5,5-tetramethyl-1,3,2-dioxa borolane (intermediate 3) in place of 2-(7-(methoxymethoxy) naphtho[1,2-b]furan-9-yl)-4,4,5,5-tetramethyl-1,3,2-dioxaborolane (intermediate 1, step 3) to afford the title compound as a white solid. HNMR (400 MHz, DMSO-d₆) δ 9.14 (s, 1H), 8.06-8.04 (m, 1H), 7.94-7.91 (m, 1H), 7.48-7.42 (m, 2H), 7.25-7.23 (m, 1H), 5.37-5.24 (m, 1H), 4.60-4.30 (m, 5H), 4.16-4.14 (m, 1H), 4.07-4.04 (m, 1H), 3.70-2.50 (m, 9H), 2.17-2.02 (m, 4H), 1.88-1.79 (m, 3H), 1.68 (m, 4H). MS: 585.5 (M+H⁺).

### Example 4

### 4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-7-(naphtho[2,1-b]furan-9-yl)pyrido[4,3-d]pyrimidine

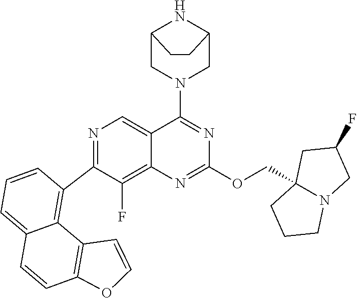 [SMILES: Fc1c(-c2cccc3ccc4occc4c23)ncc2c(N3CC4CCC(C3)N4)nc(OC[C@@]34CCCN3C[C@H](F)C4)nc12]

Example 4 was prepared essentially the same protocol described in EXAMPLE 1

With 4,4,5,5-tetramethyl-2-(naphtho[2,1-b]furan-9-yl)-1,3,2-dioxaborolane (intermediate 4) in place of 2-(7-(methoxymethoxy) naphtho[1,2-b]furan-9-yl)-4,4,5,5-tetramethyl-1,3,2-dioxaborolane (intermediate 1, step 3) to afford the title compound as a white solid. HNMR (400 MHz, DMSO-d₆) δ 9.23 (s, 1H), 8.30-8.20 (m, 1H), 8.03-8.00 (m, 1H), 7.93-7.90 (m, 1H), 7.87-7.86 (m, 1H), 7.70-7.63 (m, 2H), 5.73-5.73 (m, 1H), 5.40-5.23 (m, 1H), 4.71-4.45 (m, 2H), 4.17-4.15 (m, 1H) 4.07-4.05 (m, 1H), 3.66-3.61 (m, 4H), 3.13-3.00 (m, 3H), 2.88-2.84 (m, 1H), 2.17-2.02 (m, 4H), 1.88-1.79 (m, 3H), 1.68 (m, 4H). MS: 583.3 (M+H⁺).

### Example 5

### 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)naphtho[2,1-b]furan-7-ol

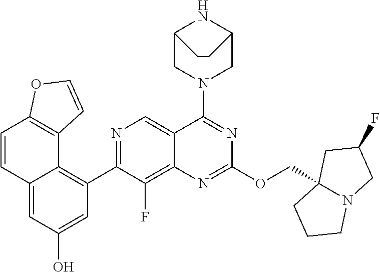 [SMILES: Oc1cc(-c2ncc3c(N4CC5CCC(C4)N5)nc(OC[C@@]45CCCN4C[C@H](F)C5)nc3c2F)c2c(ccc3occc32)c1]

Example 5 was prepared essentially the same protocol described in EXAMPLE 1

With 2-(7-(methoxymethoxy)naphtho[2,1-b]furan-9-yl)-4,4,5,5-tetramethyl-1,3,2-dioxaborolane (intermediate 5) in place of 2-(7-(methoxymethoxy) naphtho[1, 2-b]furan-9-yl)-4,4,5,5-tetramethyl-1,3,2-dioxaborolane (intermediate 1, step 3) to afford the title compound as a white solid.

¹H NMR (400 MHz, DMSO) δ 9.88 (s, 1H), 9.18 (s, 1H), 7.79-7.68 (m, 3H), 7.44 (d, J=2.5 Hz, 1H), 7.15 (d, J=2.5 Hz, 1H), 5.59 (d, J=2.1 Hz, 1H), 5.36-5.16 (m, 1H), 4.61-4.35 (m, 2H), 4.13 (d, J=10.4 Hz, 1H), 4.03 (d, J=10.4 Hz, 1H), 3.74-3.51 (m, 4H), 3.15-2.98 (m, 3H), 2.88-2.78 (m, 1H), 2.19-1.94 (m, 4H), 1.91-1.72 (m, 3H), 1.72-1.55 (m, 4H). MS: 599.6 (M+H⁺).

### Example 6

### 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-1,2-dihydronaphtho[2,1-b]furan-7-ol

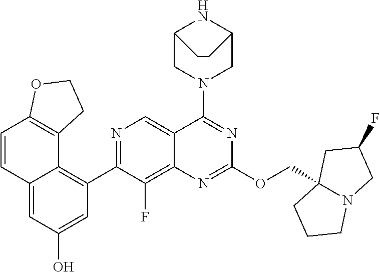 [SMILES: Oc1cc(-c2ncc3c(N4CC5CCC(C4)N5)nc(OC[C@@]45CCCN4C[C@H](F)C5)nc3c2F)c2c3c(ccc2c1)OCC3]

Example 6 was prepared essentially the same protocol described in EXAMPLE 1 With 2-(7-(methoxymethoxy)-1,2-dihydronaphtho[2,1-b]furan-9-yl)-4,4,5,5-tetra methyl-1,3,2-dioxaborolane (intermediate 6) in place of 2-(7-(methoxymethoxy) naphtho[1,2-b]furan-9-yl)-4,4,5,5-tetramethyl-1,3,2-dioxaborolane (intermediate 1, step 3) to afford the title compound as a white solid.

¹H NMR (400 MHz, DMSO) δ 9.64 (s, 1H), 9.09 (s, 1H), 7.65 (d, J=8.9 Hz, 1H), 7.27 (d, J=2.5 Hz, 1H), 7.09 (d, J=8.8 Hz, 1H), 7.02 (d, J=2.5 Hz, 1H), 5.36-5.20 (m, 1H), 4.59-4.24 (m, 4H), 4.12 (d, J=10.4 Hz, 1H), 4.03 (d, J=10.4 Hz, 1H), 3.70-3.49 (m, 4H), 3.13-2.39 (m, 6H), 2.19-1.93 (m, 4H), 1.91-1.70 (m, 3H), 1.70-1.55 (m, 4H). MS: 601.5 (M+H⁺).

### Example 7

### 9-(4-(2,5-diazabicyclo[2.2.2]octan-2-yl)-8-fluoro-2-(((2R,7aS)-2-fluorotetrahydro-1H-pyrrolizin-7a(5H)-yl)methoxy)pyrido[4,3-d]pyrimidin-7-yl)-2,3-dihydro-1H-cyclopenta[a]naphthalen-7-ol

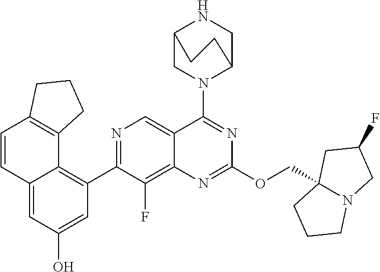 [SMILES: Oc1cc(-c2ncc3c(N4CC5CCC4CN5)nc(OC[C@@]45CCCN4C[C@H](F)C5)nc3c2F)c2c3c(ccc2c1)CCC3]

Example 7 was prepared essentially the same protocol described in EXAMPLE 2 to afford the title compound as a white solid. MS: 599.5 (M+H⁺).

### Example 8

### 9-(4-((1R,5S)-3,8-diazabicyclo[3.2.1]octan-3-yl)-2-((1-((dimethylamino)methyl)cyclopropyl)methoxy)-8-fluoropyrido[4,3-d]pyrimidin-7-yl)-2,3-dihydro-1H-cyclopenta[a]naphthalen-7-ol

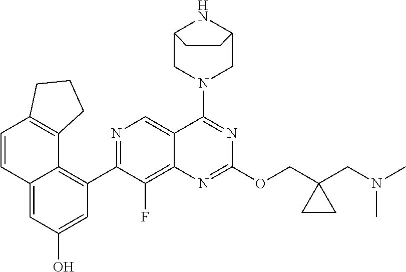 [SMILES: CN(C)CC1(COc2nc(N3CC4CCC(C3)N4)c3cnc(-c4cc(O)cc5ccc6c(c45)CCC6)c(F)c3n2)CC1]

### Step 1: (1-(((tert-butyldimethylsilyl)oxy)methyl)cyclopropyl)methanol

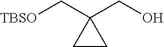 [SMILES: CC(C)(C)[Si](C)(C)OCC1(CO)CC1]

To a solution of cyclopropane-1,1-diyldimethanol (11.2 g, 110 mmol) in DMF (100 ml) were added imidazole (8.96 g, 132 mmol) and TBS-Cl (17.35 g, 115 mmol) at ice-water condition, and then the mixture was stirred at RT for 2 hs. The TLC showed the reaction was complete. Saturated NaCl was added, the following mixture was extracted with ethyl acetate three times. The combined organic layers were dried over Na₂SO₄, filtered and concentrated in vacuo to give a residual, which was purified by silica gel column and eluted with ethyl acetate/hexane from 0 to 20% to afford the title compound (14.1 g, 59.4%) as a colorless oil.

### Step 2: 1-(((tert-butyldimethylsilyl)oxy)methyl)cyclopropane-1-carbaldehyde

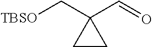 [SMILES: CC(C)(C)[Si](C)(C)OCC1(C=O)CC1]

The mixture of (1-(((tert-butyldimethylsilyl)oxy)methyl)cyclopropyl)methanol (5.5 g, 25.4 mmol), silica gel(10 g) and PCC (10.96 g, 50.8 mmol) in DCM (100 mL) was stirred at RT for 4 hours. The filtrate was concentrated in vacuo to give a residual, which was purified by silica gel column and eluted with ethyl acetate/hexane from 0 to 15% to afford the title compound (3.6 g, 66.1%) as a colorless oil.

### Step 3: 1-(1-(((tert-butyldimethylsilyl)oxy)methyl)cyclopropyl)-N,N-dimethyl methanamine

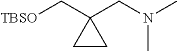 [SMILES: CN(C)CC1(CO[Si](C)(C)C(C)(C)C)CC1]

Under Ar, to a solution of 1-(((tert-butyldimethylsilyl)oxy)methyl)cyclopropane-1-carbaldehyde (500 mg, 2.332 mmol) in DCE (10 mL), 2M dimethylamine (210 mg, 4.66 mmol) in THF was added, followed by dropping catalytic amount of AcOH. Sodium triacetoxyborohydride (989 mg, 4.66 mmol) was added to the above mixture at RT. After 3 hours, saturated NaHCO₃ was added, and the resulting mixture was extracted with ethyl acetate three times. The combined organic layers were dried over Na₂SO₄, filtered and concentrated in vacuo to afford the title compound (510 mg, 90%). MS: 244.4 (M+H⁺). ¹H NMR (400 MHz, CDCl₃) δ 3.57 (s, 2H), 2.28-2.15 (m, 8H), 0.88 (s, 9H), 0.50 (t, J=5.0 Hz, 2H), 0.23 (t, J=5.0 Hz, 2H), 0.03 (s, 6H).

### Step 4: (1-((dimethylamino)methyl)cyclopropyl)methanol

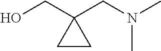

To a solution of 1-(1-(((tert-butyldimethylsilyl)oxy)methyl)cyclopropyl)-N,N-dimethylmethanamine (510 mg, 2.095 mmol) in THF (5 ml), 1M TBAF (548 mg, 2.095 mmol) in THF was added and stirred for 16 h at RT. Quenched with water, the resulting mixture was extracted with ethyl acetate three times. The combined organic layers were dried over Na₂SO₄, filtered, and concentrated in vacuo to give a residual, which was purified by silica gel column and eluted with methanol/dichloromethane from 0 to 8% to afford the title compound (160 mg, 59.1%).

¹H NMR (400 MHz, CDCl₃) δ 3.53 (s, 2H), 2.43 (s, 2H), 2.33 (s, 6H), 0.52-0.47 (m, 2H), 0.38-0.33 (m, 2H). MS: 130.1 (M+H⁺).

### Step 5: 9-(4-((1R,5S)-3,8-diazabicyclo[3.2.1]octan-3-yl)-2-((1-((dimethylamino) methyl)cyclopropyl)methoxy)-8-fluoropyrido[4,3-d]pyrimidin-7-yl)-2,3-dihydro-1H-cyclopenta[a]naphthalen-7-ol

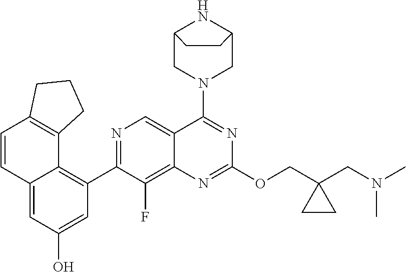 [SMILES: CN(C)CC1(COc2nc(N3CC4CCC(C3)N4)c3cnc(-c4cc(O)cc5ccc6c(c45)CCC6)c(F)c3n2)CC1]

Example 8 was prepared essentially the same protocol described in EXAMPLE 2 with (1-((dimethylamino)methyl)cyclopropyl)methanol in place of ((2R,7aS)-2-fluorotetrahydro-1H-pyrrolizin-7a(5H)-yl)methanol to afford the title compound as a white solid.

¹H NMR (400 MHz, DMSO) δ 9.80 (s, 1H), 9.08 (s, 1H), 7.62 (d, J=8.3 Hz, 1H), 7.36 (d, J=8.3 Hz, 1H), 7.27 (d, J=2.5 Hz, 1H), 6.99 (d, J=2.5 Hz, 1H), 4.48 (d, J=11.0 Hz, 1H), 4.36 (d, J=11.5 Hz, 1H), 4.24 (s, 2H), 3.64 (d, J=11.8 Hz, 1H), 3.58-3.49 (m, 3H), 2.86 (t, J=7.4 Hz, 2H), 2.25-2.14 (m, 10H), 2.06-1.91 (m, 1H), 1.84-1.72 (m, 2H), 1.69-1.59 (m, 4H), 0.63 (t, J=5.0 Hz, 2H), 0.40 (t, J=5.1 Hz, 2H). MS: 569.4 (M+H⁺).

### Example 9

### 9-(4-((1R,5S)-3,8-diazabicyclo[3.2.1]octan-3-yl)-8-fluoro-2-((1-(morpholinomethyl)cyclopropyl)methoxy)pyrido[4,3-d]pyrimidin-7-yl)-2,3-dihydro-1H-cyclopenta[a]naphthalen-7-ol

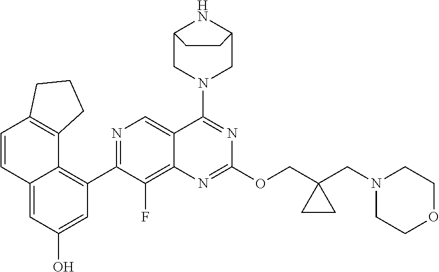 [SMILES: Oc1cc(-c2ncc3c(N4CC5CCC(C4)N5)nc(OCC4(CN5CCOCC5)CC4)nc3c2F)c2c3c(ccc2c1)CCC3]

### Step 1: 4-((1-(((tert-butyldimethylsilyl)oxy)methyl)cyclopropyl)methyl) morpholine

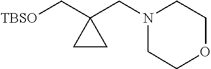 [SMILES: CC(C)(C)[Si](C)(C)OCC1(CN2CCOCC2)CC1]

To a solution of 1-(((tert-butyldimethylsilyl)oxy)methyl)cyclopropane-1-carbaldehyde (1.5 g, 7.00 mmol) in DCE (10 ml), morpholine(0.792 g, 9.10 mmol), followed by dropping catalytic amount of AcOH. Sodium triacetoxyborohydride (2.97 g, 13.99 mmol) was added to the mixture at RT. After stirred at RT for 3 hours. saturated NaHCO₃ was added, and the resulting mixture was extracted with ethyl acetate three times. The combined organic layers were dried over Na₂SO₄, filtered and concentrated in vacuo to afford the title compound (2 g, 100%). MS: 286.4 (M+H⁺).

### Step 2: (1-(morpholinomethyl)cyclopropyl)methanol

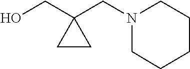 [SMILES: OCC1(CN2CCCCC2)CC1]

To a solution of 4-((1-(((tert-butyldimethylsilyl)oxy)methyl)cyclopropyl)methyl) morpholine (2 g, 7.01 mmol) in THF (20 mL), 1M TBAF (1.832 g, 7.01 mmol) in THF was added. After stirred at rt for 16 hours. and the mixture was stirred for 16 h. Quenched with water, the resulting mixture was extracted with ethyl acetate three times. The combined organic layers were dried over Na₂SO₄, filtered, and concentrated in vacuo to give a residual, which was purified by silica gel column and eluted with methanol/dichloromethane from 0 to 8% to afford the title compound (690 mg, 57.5%). ¹H NMR (400 MHz, CDCl₃) δ 3.71 (t, J=4.6 Hz, 4H), 3.53 (s, 2H), 2.58 (brs, 4H), 2.47 (s, 2H), 0.50 (t, J=5.2 Hz, 2H), 0.35 (t, J=5.3 Hz, 2H). MS: 172.2 (M+H⁺)

### Step 3: 9-(4-((1R,5S)-3,8-diazabicyclo[3.2.1]octan-3-yl)-8-fluoro-2-((1-(morpholinomethyl)cyclopropyl)methoxy)pyrido[4,3-d]pyrimidin-7-yl)-2,3-dihydro-1H-cyclopenta[a]naphthalen-7-ol

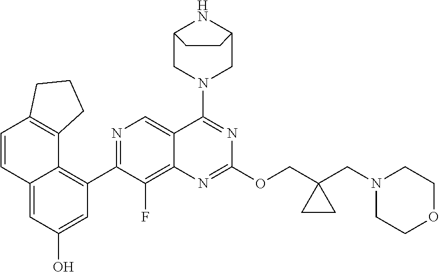 [SMILES: Oc1cc(-c2ncc3c(N4CC5CCC(C4)N5)nc(OCC4(CN5CCOCC5)CC4)nc3c2F)c2c3c(ccc2c1)CCC3]

Example 9 was prepared essentially the same protocol described in EXAMPLE 2 with (1-(morpholinomethyl)cyclopropyl)methanol in place of ((2R,7aS)-2-fluoro tetrahydro-1H-pyrrolizin-7a(5H)-yl)methanol to afford the title compound as a white solid.

¹H NMR (400 MHz, DMSO) δ 9.79 (s, 1H), 9.09 (s, 1H), 7.63 (d, J=8.3 Hz, 1H), 7.36 (d, J=8.3 Hz, 1H), 7.28 (d, J=2.5 Hz, 1H), 6.99 (d, J=2.5 Hz, 1H), 4.56-4.19 (m, 4H), 3.69-3.46 (m, 8H), 2.86 (t, J=7.4 Hz, 2H), 2.43-2.32 (m, 4H), 2.32-2.11 (m, 4H), 2.03-1.94 (m, 1H), 1.85-1.72 (m, 2H), 1.69-1.56 (m, 4H), 0.64 (t, J=4.9 Hz, 2H), 0.41 (t, J=5.1 Hz, 2H). MS. 611.6 (M+H⁺).

### Example 10

### 9-(2-(3-(3-oxa-8-azabicyclo[3.2.1]octan-8-yl)propoxy)-4-((1R,5S)-3,8-diazabicyclo[3.2.1]octan-3-yl)-8-fluoropyrido[4,3-d]pyrimidin-7-yl)-2,3-dihydro-1H-cyclopenta[a]naphthalen-7-ol

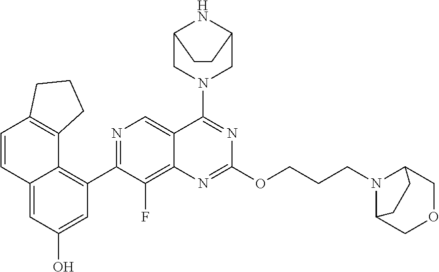 [SMILES: Oc1cc(-c2ncc3c(N4CC5CCC(C4)N5)nc(OCCCN4C5CCC4COC5)nc3c2F)c2c3c(ccc2c1)CCC3]

Example 10 was prepared essentially the same protocol described in EXAMPLE 2 with 3-(3-oxa-8-azabicyclo[3.2.1]octan-8-yl)propan-1-ol in place of ((2R,7aS)-2-fluoro tetrahydro-1H-pyrrolizin-7a(5H)-yl)methanol to afford the title compound as a white solid. HNMR: (CDCl₃, 400 MHz): 9.00 (s, 1H), 7.71 (d, J=8.4 Hz, 1H), 7.32 (d, J=8.4 Hz, 1H), 7.21-7.06 (m, 2H), 4.58-4.47 (m, 4H), 3.74-3.48 (m, 8H), 3.09-2.89 (m, 4H), 2.53-2.49 (m, 2H), 2.42-2.37 (m, 1H), 2.28-2.22 (m, 1H), 2.03-1.81 (m, 13H). MS: 611.6 (M+H⁺).

### Example 11: 9-(4-((1R,5S)-3,8-diazabicyclo[3.2.1]octan-3-yl)-8-fluoro-2-((1-(((R)-3-methylmorpholino)methyl)cyclopropyl)methoxy)pyrido[4,3-d]pyrimidin-7-yl)-2,3-dihydro-1H-cyclopenta[a]naphthalen-7-ol

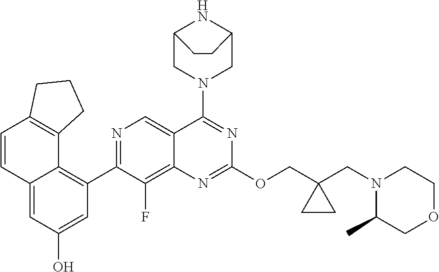 [SMILES: C[C@@H]1COCCN1CC1(COc2nc(N3CC4CCC(C3)N4)c3cnc(-c4cc(O)cc5ccc6c(c45)CCC6)c(F)c3n2)CC1]

Example 11 was prepared essentially the same protocol described in EXAMPLE 2 with (R)-(1-((3-methylmorpholino)methyl)cyclopropyl)methanol in place of ((2R,7aS)-2-fluoro tetrahydro-1H-pyrrolizin-7a(5H)-yl)methanol to afford the title compound as a white solid. HNMR(DMSO, 400 MHz): 9.78 (s, 1H), 9.08 (s, 1H), 9.63 (d, J=8.4 Hz, 1H), 7.36 (d, J=8.4 Hz, 1H), 7.27 (d, J=2.0 Hz, 1H), 6.99 (d, J=2.0 Hz, 1H), 4.66-4.32 (m, 3H), 4.05-3.96 (m, 1H), 3.68-3.42 (m, 7H), 3.29-3.19 (m, 2H), 2.97-2.84 (m, 4H), 2.33-2.01 (m, 4H), 1.79-1.54 (m, 7H), 0.810-0.78 (m, 3H), 0.71-0.65 (m, 1H), 0.58-0.46 (m, 2H), 0.35-0.31 (m, 1H). MS: 625.6 (M+H⁺).

### Example 12

### 9-(2-((2R)-3-(3-oxa-8-azabicyclo[3.2.1]octan-8-yl)-2-methylpropoxy)-4-((1R,5S)-3,8-diazabicyclo[3.2.1]octan-3-yl)-8-fluoropyrido[4,3-d]pyrimidin-7-yl)-2,3-dihydro-1H-cyclopenta[a]naphthalen-7-ol

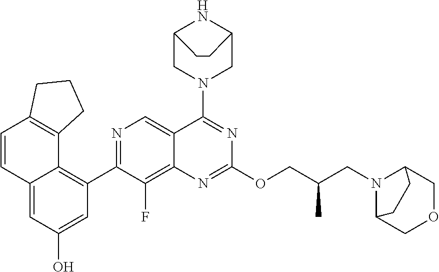 [SMILES: C[C@@H](COc1nc(N2CC3CCC(C2)N3)c2cnc(-c3cc(O)cc4ccc5c(c34)CCC5)c(F)c2n1)CN1C2CCC1COC2]

Example 12 was prepared essentially the same protocol described in EXAMPLE 2 with (2R)-3-(3-oxa-8-azabicyclo[3.2.1]octan-8-yl)-2-methylpropan-1-ol in place of ((2R,7aS)-2-fluoro tetrahydro-1H-pyrrolizin-7a(5H)-yl)methanol to afford the title compound as a white solid. HNMR(DMSO, 400 MHz): 9.78 (s, 1H), 9.11 (s, 1H), 7.63 (d, J=8.0 Hz, 1H), 7.36 (d, J=8.4 Hz, 1H), 7.28 (d, J=2.4 Hz, 1H), 6.98 (d, J=2.4 Hz, 1H), 4.59-4.42 (m, 3H), 4.26-4.19 (m, 1H), 3.78-3.36 (m, 8H), 3.04-2.84 (m, 5H), 2.68-2.66 (m, 1H), 2.33-2.18 (m, 4H), 1.82-1.67 (m, 10H), 1.01 (d, J=6.8 Hz, 3H). MS: 625.5 (M+H⁺).

### Example 13

### 1-(8-fluoro-2-(((2R)-2-fluorotetrahydro-1H-pyrrolizin-7a(5H)-yl)methoxy)-7-(7-hydroxy-2,3-dihydro-1H-cyclopenta[a]naphthalen-9-yl)pyrido[4,3-d]pyrimidin-4-yl)-3-methylpiperidin-3-ol

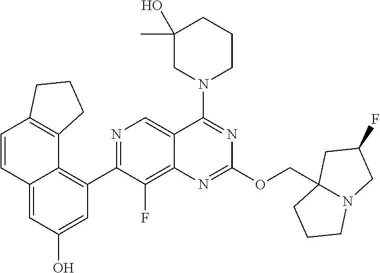 [SMILES: CC1(O)CCCN(c2nc(OCC34CCCN3C[C@H](F)C4)nc3c(F)c(-c4cc(O)cc5ccc6c(c45)CCC6)ncc23)C1]

Example 13 was prepared essentially the same protocol described in EXAMPLE 1 (step 1) with 3-methylpiperidin-3-ol in place of tert-butyl (1R,5S)-3,8-diazabicyclo[3.2.1]octane-8-carboxylate to afford the title compound as a white solid. HNMR(DMSO, 400 MHz): 9.77 (s, 1H), 9.23 (s, 1H), 7.63 (d, J=8.4 Hz, 1H), 7.36 (d, J=8.4 Hz, 1H), 7.28 (d, J=2.0 Hz, 1H), 7.00 (d, J=2.0 Hz, 1H), 5.34-5.21 (m, 1H), 4.79-4.70 (m, 1H), 4.34-4.31 (m, 1H), 4.16-4.03 (m, 3H), 3.65-3.51 (m, 1H), 3.41-3.33 (m, 1H), 3.11-3.02 (m, 3H), 2.88-2.80 (m, 3H), 2.22-2.00 (m, 6H), 1.82-1.65 (m, 8H), 1.18 (s, 3H). MS: 602.5 (M+H⁺).

### Example 14

### 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-1-methyl-2,3-dihydro-1H-cyclopenta[a]naphthalen-7-ol

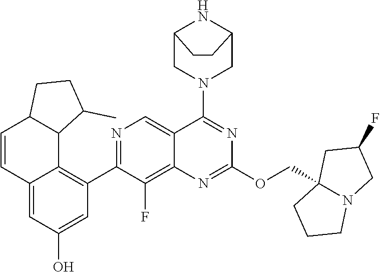 [SMILES: CC1CCC2C=Cc3cc(O)cc(-c4ncc5c(N6CC7CCC(C6)N7)nc(OC[C@@]67CCCN6C[C@H](F)C7)nc5c4F)c3C12]

Example 14 was prepared essentially the same protocol described in EXAMPLE 2 (step 1) with 2-(7-(methoxymethoxy)-1-methyl-2,3-dihydro-1H cyclopenta[a]naphthalen-9-yl)-4,4,5,5-tetramethyl-1,3,2-dioxaborolane (Intermediate 7) in place of 2-(7-(methoxymethoxy)-2,3-dihydro-1H-cyclopenta[a]naphthalen-9-yl)-4,4,5,5-tetramethyl-1,3,2-dioxaborolane to afford the title compound as a white solid. MS: 613.6 (M+H⁺).

### Example 15

### 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)naphtho[1,2-b]thiophen-7-ol

Molecular Weight: 614.72

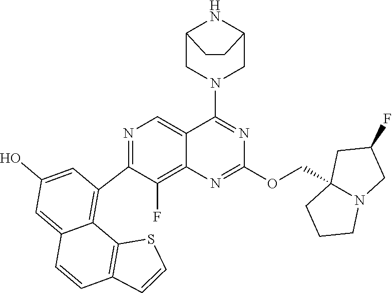 [SMILES: Oc1cc(-c2ncc3c(N4CC5CCC(C4)N5)nc(OC[C@@]45CCCN4C[C@H](F)C5)nc3c2F)c2c(ccc3ccsc32)c1]

Example 15 was prepared essentially the same protocol described in EXAMPLE 2 (step 1) with 2-(7-(methoxymethoxy)naphtho[1,2-b]thiophen-9-yl)-4,4,5,5-tetramethyl-1,3,2-dioxaborolane (Intermediate 8) in place of 2-(7-(methoxymethoxy)-2,3-dihydro-1H-cyclopenta[a]naphthalen-9-yl)-4,4,5,5-tetramethyl-1,3,2-dioxaborolane to afford the title compound as a white solid.

HNMR(DMSO, 400 MHz): 10.04 (s, 1H), 9.21 (s, 1H), 7.88 (d, J=8.8 Hz, 1H), 7.76 (d, J=8.8 Hz, 1H), 7.45-7.42 (m, 3H), 7.19-6.65 (m, 2H), 5.33-5.21 (m, 2H), 4.54-4.43 (m, 2H), 4.14-4.03 (m, 2H), 3.09-2.80 (m, 4H), 2.18-1.97 (m, 6H), 1.85-1.70 (m, 6H), 1.49-1.42 (m, 1H). MS: 615.5 (M+H⁺).

### Example 16

### 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5,6,7,8-tetrahydrophenanthren-2-ol

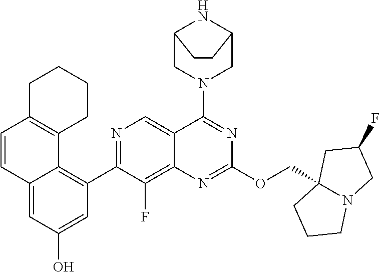 [SMILES: Oc1cc(-c2ncc3c(N4CC5CCC(C4)N5)nc(OC[C@@]45CCCN4C[C@H](F)C5)nc3c2F)c2c3c(ccc2c1)CCCC3]

Example 16 was prepared essentially the same protocol described in EXAMPLE 2 (step 1) with 2-(2-(methoxymethoxy)-5,6,7,8-tetrahydrophenanthren-4-yl)-4,4,5,5-tetramethyl-1,3,2-dioxaborolane (Intermediate 9) in place of 2-(7-(methoxymethoxy)-2,3-dihydro-1H-cyclopenta[a]naphthalen-9-yl)-4,4,5,5-tetramethyl-1,3,2-dioxaborolane to afford the title compound as a white solid. MS: 613.7 (M+H⁺).

### Example 17

### 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-7-hydroxy-2,3-dihydro-1H-cyclopenta[a]naphthalen-1-one

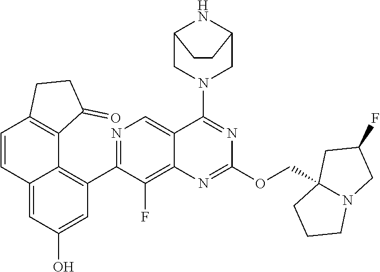 [SMILES: O=C1CCc2ccc3cc(O)cc(-c4ncc5c(N6CC7CCC(C6)N7)nc(OC[C@@]67CCCN6C[C@H](F)C7)nc5c4F)c3c21]

Example 17 was prepared essentially the same protocol described in EXAMPLE 2 (step 1) with 7-(methoxymethoxy)-9-(4,4,5,5-tetramethyl-1,3,2-dioxaborolan-2-yl)-2,3-dihydro-1H-cyclopenta[a]naphthalen-1-one (Intermediate 10) in place of 2-(7-(methoxymethoxy)-2,3-dihydro-1H-cyclopenta[a]naphthalen-9-yl)-4,4,5,5-tetramethyl-1,3,2-dioxaborolane to afford the title compound as a white solid.

HNMR(DMSO, 400 MHz): 10.14 (s, 1H), 8.95 (s, 1H), 8.12 (d, J=8.4 Hz, 1H), 7.59 (d, J=8.4 Hz, 1H), 7.42 (d, J=2.0 Hz, 1H), 7.34 (d, J=2.0 Hz, 1H), 5.34-5.21 (m, 1H), 4.46 (d, J=11.2 Hz, 1H), 4.30 (d, J=2.0 Hz, 1H), 4.12 (d, J=10.4 Hz, 1H), 4.02 (d, J=10.4 Hz, 1H), 3.64-3.51 (m, 4H), 3.13-3.01 (m, 5H), 2.85-2.80 (m, 1H), 2.53-2.40 (m, 3H), 2.15-1.97 (m, 3H), 1.85-1.60 (m, 7H). MS: 613.7 (M+H⁺).

### Example 18

### 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-2-(hydroxymethyl)-2,3-dihydro-1H-cyclopenta[a]naphthalen-7-ol

Molecular Weight: 628.72

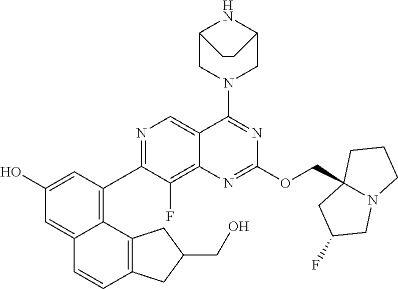 [SMILES: OCC1Cc2ccc3cc(O)cc(-c4ncc5c(N6CC7CCC(C6)N7)nc(OC[C@@]67CCCN6C[C@H](F)C7)nc5c4F)c3c2C1]

### Step 1: tert-butyl (1R,5S)-3-(7-(2-(ethoxycarbonyl)-7-(methoxymethoxy)-2,3-dihydro-1H-cyclopenta[a]naphthalen-9-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate

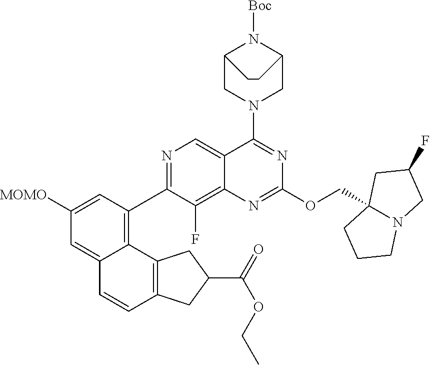

Under Ar, the mixture of tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (41.4 mg, 0.075 mmol), ethyl 7-(methoxymethoxy)-9-(4,4,5,5-tetramethyl-1,3,2-dioxaborolan-2-yl)-2,3-dihydro-1H-cyclopenta[a]naphthalene-2-carboxylate (32 mg, 0.075 mmol), Ruphos-Pd-G3 (9.43 mg, 0.011 mmol) and K₃PO₄ (80 mg, 0.375 mmol) in THF (5 mL)/Water (3 mL) was stirred at 60° C. for 2 h. After cooling down to RT, the mixture was partioned between EA/Water, the separated organic layer was concentrated in vacuo to give a residual, which was purified by silica gel column and eluted with MeOH/DCM=19:1 to afford the desired compound (53 mg, 87%) as a colorless oil. MS: 815.8 (M+H⁺).

### Step 2: tert-butyl (1R,5S)-3-(8-fluoro-2-(((2R,7aS)-2-fluorotetrahydro-1H-pyrrolizin-7a(5H)-yl)methoxy)-7-(2-(hydroxymethyl)-7-(methoxymethoxy)-2,3-dihydro-1H-cyclopenta[a]naphthalen-9-yl)pyrido[4,3-d]pyrimidin-4-yl)-3,8-diazabicyclo[3.2.1]octane-8-carboxylate

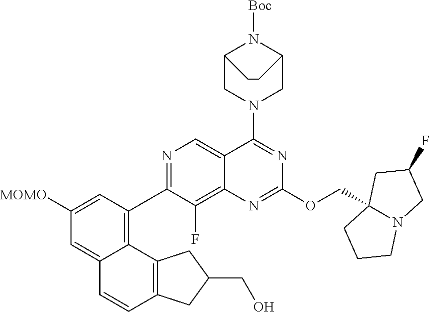

Under Ar, to a solution of tert-butyl (1R,5S)-3-(7-(2-(ethoxycarbonyl)-7-(methoxymethoxy)-2,3-dihydro-1H-cyclopenta[a]naphthalen-9-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (50 mg, 0.061 mmol) in dry DCM (5 mL) was added DIBAL-H (17.45 mg, 0.123 mmol) at −78° C., then the mixture was stirred at −78° C. for 1 h. Quenched with saturated NH₄Cl solution, and the resulting mixture was extracted with DCM for three times, the combined organic layers were washed with brine, dried over Na₂SO₄, concentrated in vacuo to give a residual, which was purified by silica gel column and eluted with MeOH/DCM=19:1 to afford the desired compound (23 mg, 48.5%) as a colorless oil. MS: 773.7 (M+H⁺).

### Step 3: 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-2-(hydroxymethyl)-2,3-dihydro-1H-cyclopenta[a]naphthalen-7-ol

Molecular Weight: 628.72

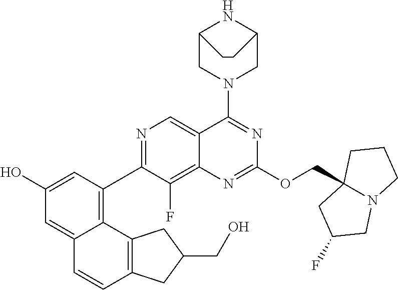 [SMILES: OCC1Cc2ccc3cc(O)cc(-c4ncc5c(N6CC7CCC(C6)N7)nc(OC[C@@]67CCCN6C[C@H](F)C7)nc5c4F)c3c2C1]

Under Ar, the mixture of tert-butyl (1R,5S)-3-(8-fluoro-2-(((2R,7aS)-2-fluorotetrahydro-1H-pyrrolizin-7a(5H)-yl)methoxy)-7-(2-(hydroxymethyl)-7-(methoxymethoxy)-2,3-dihydro-1H-cyclopenta[a]naphthalen-9-yl)pyrido[4,3-d]pyrimidin-4-yl)-3,8-diazabicyclo[3.2.1]octane-8-carboxylate (23 mg, 0.030 mmol) and HCl/dioxane(1N, 5 mL) was stirred at rt for 2 h. The volatiles was removed under reduced pressure to give a residue, which was partioned in DCM and saturated NaHCO₃ aq, the separated organic layer was washed with brine. dried over Na₂SO₄, filtered, the filtrate was concentrated in vacuo to give a residue, which was purified by Pre-HPLC to afford the desired compound (3.1 mg, 16.57%) as a white solid. HNMR(DMSO, 400 MHz): 9.77 (s, 1H), 9.10 (d, J=4.8 Hz, 1H), 7.62 (d, J=8.4 Hz, 1H), 7.32 (d, J=8.4 Hz, 1H), 7.27 (d, J=2.0 Hz, 1H), 6.98 (dd, J=10.8 Hz, 2.0 Hz, 1H), 5.35-5.31 (m, 1H), 4.53-4.38 (m, 2H), 4.15-4.04 (m, 2H), 3.80-3.67 (m, 3H), 3.23-2.83 (m, 9H), 2.67-2.63 (m, 1H), 2.33-1.97 (m, 7H), 1.91-1.69 (m, 7H). MS: 629.6 (M+H⁺).

### Example 19

### 5-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-2,3-dihydro-1H-pyrrolo[1,2-a]indol-7-ol

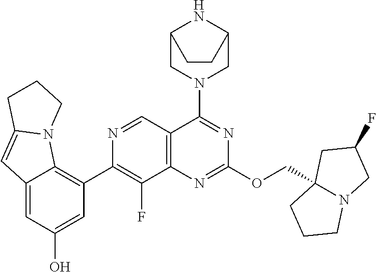 [SMILES: Oc1cc(-c2ncc3c(N4CC5CCC(C4)N5)nc(OC[C@@]45CCCN4C[C@H](F)C5)nc3c2F)c2c(c1)cc1n2CCC1]

### Step 1: tert-butyl (1R,5S)-3-(7-(7-((tert-butyldimethylsilyl)oxy)-2,3-dihydro-1H-pyrrolo[1,2-a]indol-5-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate

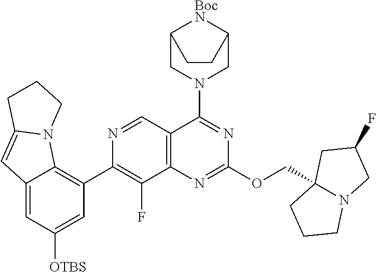 [SMILES: CC(C)(C)OC(=O)N1C2CCC1CN(c1nc(OC[C@@]34CCCN3C[C@H](F)C4)nc3c(F)c(-c4cc(O[Si](C)(C)C(C)(C)C)cc5cc6n(c45)CCC6)ncc13)C2]

Under Ar, the mixture of tert-butyl (1R,5S)-3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (50 mg, 0.09 mmol), 7-((tert-butyldimethylsilyl)oxy)-5-(4,4,5,5-tetramethyl-1,3,2-dioxaborolan-2-yl)-2,3-dihydro-1H-pyrrolo[1,2-a]indole (56 mg, 0.14 mmol), K₃PO₄ (58 mg, 0.27 mmol) and Ruphos Pd G3 (7.6 mg, 0.01 mmol) in THF (5 mL)/Water (1 mL) was stirred at 60° C. for 1 h. After cooling down to RT, water was added, the resulting mixture was extracted with ethyl acetate three times. The combined organic layers were washed with brine, dried by anhydrous sodium sulfate, concentrated in vacuo to give a residual, which was purified by silica gel column and eluted with Methanol/DCM from 0 to 10% to afford the desired compound (70.0 mg, 96%) as a yellow solid. MS: 802.6 (M+H⁺).

### Step 2: 5-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-2,3-dihydro-1H-pyrrolo[1,2-a]indol-7-ol

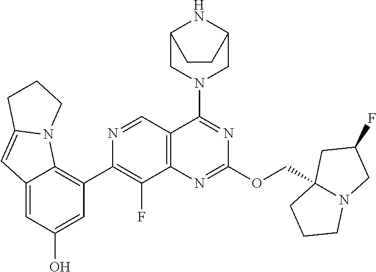 [SMILES: Oc1cc(-c2ncc3c(N4CC5CCC(C4)N5)nc(OC[C@@]45CCCN4C[C@H](F)C5)nc3c2F)c2c(c1)cc1n2CCC1]

Under Ar, the mixture of tert-butyl (1R,5S)-3-(7-(7-((tert-butyldimethylsilyl)oxy)-2,3-dihydro-1H-pyrrolo[1,2-a]indol-5-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (70 mg, 0.87 mmol) in ACN (2 mL) and 4N HCl in dioxane (0.5 ml) was stirred at RT for 0.5 h. The volatiles was removed in vacuo to give a residual, which was purified by pre-HPLC to afford the desired compound (3.5 mg, 7%) as a white solid. H NMR (400 MHz, DMSO) δ 9.16 (s, 1H), 8.85 (s, 1H), 6.94-6.94 (d, J=2.0 Hz, 1H), 6.67 (s, 1H), 6.09 (s, 1H), 5.45-5.25 (m, 1H), 4.51-4.48 (d, J=10 Hz, 2H), 4.18-4.15 (d, J=10.4 Hz, 1H), 4.08-4.05 (d, J=10.4 Hz, 1H), 3.71-3.67 (m, 4H), 3.57-3.53 (m, 2H), 3.13-3.05 (m, 3H), 2.91-2.87 (m, 3H), 2.37-1.72 (m, 13H). MS: 588.5 (M+H⁺)

### Example 20

### 5-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-9-chloro-2,3-dihydro-1H-pyrrolo[1,2-a]indol-7-ol

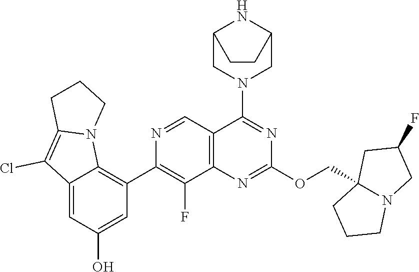 [SMILES: Oc1cc(-c2ncc3c(N4CC5CCC(C4)N5)nc(OC[C@@]45CCCN4C[C@H](F)C5)nc3c2F)c2c(c1)c(Cl)c1n2CCC1]

### Step 1: tert-butyl (1R,5S)-3-(7-(7-((tert-butyldimethylsilyl)oxy)-9-chloro-2,3-dihydro-1H-pyrrolo[1,2-a]indol-5-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate

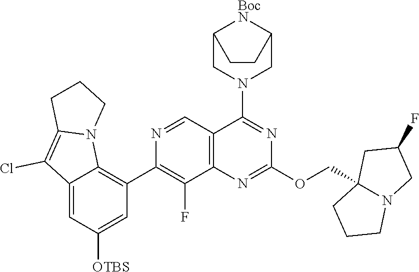 [SMILES: CC(C)(C)OC(=O)N1C2CCC1CN(c1nc(OC[C@@]34CCCN3C[C@H](F)C4)nc3c(F)c(-c4cc(O[Si](C)(C)C(C)(C)C)cc5c(Cl)c6n(c45)CCC6)ncc13)C2]

Under Ar, the mixture of tert-butyl (1R,5S)-3-(7-(7-((tert-butyldimethylsilyl)oxy)-2,3-dihydro-1H-pyrrolo[1,2-a]indol-5-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (90 mg, 0.112 mmol) and NCS (15 mg, 0.112 mmol) in DMF (2 mL) was stirred at room temperature for 1 h. Quenched with water, the resulting mixture was extracted with ethyl acetate three times. The combined organic layers were washed with brine, dried over sodium sulfate, concentrated in vacuo to give a residual, which was purified by silica gel column and eluted with EA/Hex from 0 to 10% to afford the desired compound (50.0 mg, 53%) as a white solid. MS: 836.7 (M+H⁺).

### Step 2: 5-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-9-chloro-2,3-dihydro-1H-pyrrolo[1,2-a]indol-7-ol

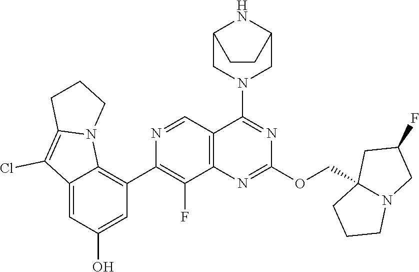 [SMILES: Oc1cc(-c2ncc3c(N4CC5CCC(C4)N5)nc(OC[C@@]45CCCN4C[C@H](F)C5)nc3c2F)c2c(c1)c(Cl)c1n2CCC1]

Under Ar, the mixture of tert-butyl (1R,5S)-3-(7-(7-((tert-butyldimethylsilyl)oxy)-9-chloro-2,3-dihydro-1H-pyrrolo[1,2-a]indol-5-yl)-8-fluoro-2-(((2R,7aS)-2-fluorotetrahydro-1H-pyrrolizin-7a(5H)-yl)methoxy)pyrido[4,3-d]pyrimidin-4-yl)-3,8-diazabicyclo[3.2.1]octane-8-carboxylate (50 mg, 0.06 mmol) in ACN (2 mL) and 4N HCl in dioxane (0.5 ml) was stirred at RT for 0.5 h. The volatiles was removed in vacuo to give a residual, which was purified by pre-HPLC to afford the desired compound (14 mg, 38%) as a white solid. H NMR (400 MHz, DMSO) δ 9.19 (s, 1H), 9.15 (s, 1H), 6.88-6.87 (d, J=2.4 Hz, 1H), 6.78 (s, 1H), 5.37-5.24 (m, 1H), 4.45-4.42 (d, J=10 Hz, 2H), 4.16-4.14 (d, J=10.4 Hz, 1H), 4.06-4.04 (d, J=10.4 Hz, 1H), 3.64-3.56 (m, 6H), 3.13-3.05 (m, 3H), 2.94-2.85 (m, 3H), 2.40-1.62 (m, 13H). MS: 622.5 (M+H⁺).

### Example 21: 9-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-1,3-dihydronaphtho[1,2-c]furan-7-ol

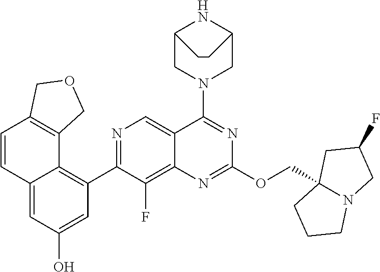 [SMILES: Oc1cc(-c2ncc3c(N4CC5CCC(C4)N5)nc(OC[C@@]45CCCN4C[C@H](F)C5)nc3c2F)c2c3c(ccc2c1)COC3]

Example 21 was prepared essentially the same protocol described in EXAMPLE 2 (step 1) with 2-(7-(benzyloxy)-1,3-dihydronaphtho[1,2-c]furan-9-yl)-4,4,5,5-tetramethyl-1,3,2-dioxaborolane (Intermediate 13) in place of 2-(7-(methoxymethoxy)-2,3-dihydro-1H-cyclopenta[a]naphthalen-9-yl)-4,4,5,5-tetramethyl-1,3,2-dioxaborolane to afford the title compound as a white solid.

¹H NMR (400 MHz, DMSO) δ 9.97 (s, 1H), 9.08 (s, 1H), 7.79 (d, J=8.4 Hz, 1H), 7.39 (m, 2H), 7.08 (d, J=2.4 Hz, 1H), 5.33-5.19 (m, 1H), 5.00 (s, 2H), 4.42 (m, 2H), 4.29 (s, 2H), 4.10 (m, 2H), 3.58 (m, 4H), 3.07 (m, 3H), 2.81 (m, 1H), 2.14-1.90 (m, 4H), 1.84-1.73 (m, 3H), 1.70-1.60 (m, 4H). MS: 601.6 (M+H⁺)

### Example 22

### 10-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)pyrrolo[2,1-a]isoquinolin-8-ol

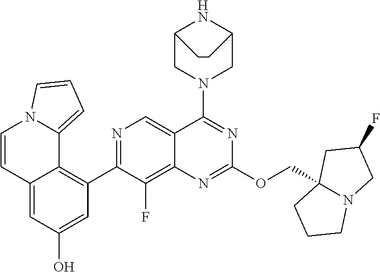 [SMILES: Oc1cc(-c2ncc3c(N4CC5CCC(C4)N5)nc(OC[C@@]45CCCN4C[C@H](F)C5)nc3c2F)c2c(ccn3cccc23)c1]

Example 22 was prepared essentially the same protocol described in EXAMPLE 2 (step 1) with 8-(methoxymethoxy)-10-(4,4,5,5-tetramethyl-1,3,2-dioxaborolan-2-yl)pyrrolo[2,1-a]isoquinoline (Intermediate 14) in place of 2-(7-(methoxymethoxy)-2,3-dihydro-1H-cyclopenta[a]naphthalen-9-yl)-4,4,5,5-tetramethyl-1,3,2-dioxaborolane to afford the title compound as a white solid.

¹H NMR (400 MHz, DMSO) δ 9.87 (s, 1H), 9.20 (s, 1H), 8.10 (d, J=7.2 Hz, 1H), 7.38 (m, 2H), 7.14 (d, J=2.4 Hz, 1H), 6.87 (d, J=2.4 Hz, 1H), 6.84 (d, J=7.2 Hz, 1H), 5.36-5.23 (m, 1H), 5.02-5.01 (m, 1H), 4.54-4.43 (m, 2H), 4.16-4.06 (m, 2H), 3.70-3.60 (m, 4H), 3.12-2.80 (m, 5H), 2.20-1.60 (m, 10H). MS: 598.5 (M+H⁺).

### Example 23

### 9-(2-((1-((3-oxa-8-azabicyclo[3.2.1]octan-8-yl)methyl)cyclopropyl)methoxy)-4-((1R,5S)-3,8-diazabicyclo[3.2.1]octan-3-yl)-8-fluoropyrido[4,3-d]pyrimidin-7-yl)-2,3-dihydro-1H-cyclopenta[a]naphthalen-7-ol

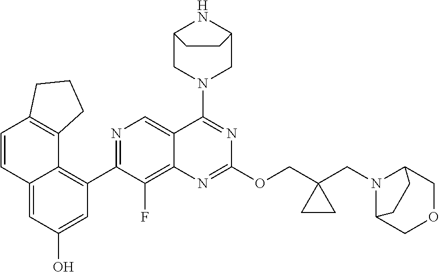 [SMILES: Oc1cc(-c2ncc3c(N4CC5CCC(C4)N5)nc(OCC4(CN5C6CCC5COC6)CC4)nc3c2F)c2c3c(ccc2c1)CCC3]

Example 23 was prepared essentially the same protocol described in EXAMPLE 2 with (1-((3-oxa-8-azabicyclo[3.2.1]octan-8-yl)methyl)cyclopropyl)methanol in place of ((2R,7aS)-2-fluoro tetrahydro-1H-pyrrolizin-7a(5H)-yl)methanol to afford the title compound as a white solid. MS: 637.5 (M+H⁺).

### Example 24

### 9-(4-((1R,5S)-3,8-diazabicyclo[3.2.1]octan-3-yl)-8-fluoro-2-((1-((tetrahydro-1H-furo[3,4-c]pyrrol-5 (3H)-yl)methyl)cyclopropyl)methoxy)quinazolin-7-yl)-2,3-dihydro-1H-cyclopenta[a]naphthalen-7-ol

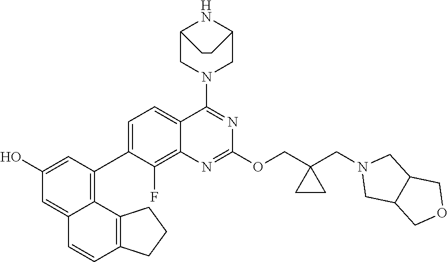 [SMILES: Oc1cc(-c2ccc3c(N4CC5CCC(C4)N5)nc(OCC4(CN5CC6COCC6C5)CC4)nc3c2F)c2c3c(ccc2c1)CCC3]

### Step 1: tert-butyl (1R,5S)-3-(8-fluoro-7-(7-(methoxymethoxy)-2,3-dihydro-1H-cyclopenta[a]naphthalen-9-yl)-2-((1-((tetrahydro-1H-furo[3,4-c]pyrrol-5 (3H)-yl)methyl)cyclopropyl)methoxy)quinazolin-4-yl)-3,8-diazabicyclo[3.2.1]octane-8-carboxylate

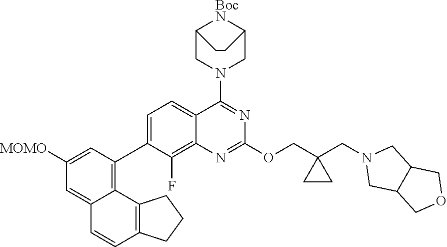

Under Ar, the mixture of (1-((tetrahydro-1H-furo[3,4-c]pyrrol-5 (3H)-yl)methyl)cyclopropyl)methanol (42 mg, 0.2 mmol), NaH (8.8 mg, 2.2 mmol, 60% purity in paraffin) and 5 mL dry THF was stirred at room temperature for 0.5 h. Then tert-butyl (1R,5S)-3-(2,8-difluoro-7-(7-(methoxymethoxy)-2,3-dihydro-1H-cyclopenta[a]naphthalen-9-yl)quinazolin-4-yl)-3,8-diazabicyclo[3.2.1]octane-8-carboxylate (60 mg, 0.1 mmol) was added to the reaction mixture and stirred for another 0.5 h. After the reaction completed, saturated ammonium chloride solution was added to quench the reaction and the resulting mixture was extracted with EA for 3 times. The combined organic layers were washed with brine, dried over anhydrous Na₂SO₄ and concentrated in vacuo to give a residual, which was purified by silica gel column and eluted with DCM/MeOH (20:1) to afford the title compound (16.7 mg, 21% yield). MS: 780.5 (M+H⁺).

### Step 2: 9-(4-((1R,5S)-3,8-diazabicyclo[3.2.1]octan-3-yl)-8-fluoro-2-((1-((tetrahydro-1H-furo[3,4-c]pyrrol-5 (3H)-yl)methyl)cyclopropyl)methoxy)quinazolin-7-yl)-2,3-dihydro-1H-cyclopenta[a]naphthalen-7-ol

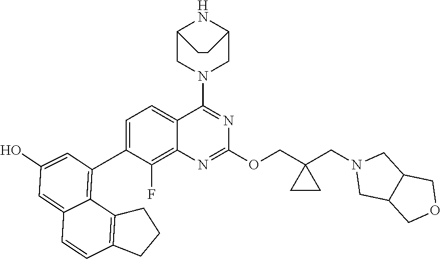 [SMILES: Oc1cc(-c2ccc3c(N4CC5CCC(C4)N5)nc(OCC4(CN5CC6COCC6C5)CC4)nc3c2F)c2c3c(ccc2c1)CCC3]

Under Ar, the mixture of tert-butyl (1R,5S)-3-(8-fluoro-7-(7-(methoxymethoxy)-2,3-dihydro-1H-cyclopenta[a]naphthalen-9-yl)-2-((1-((tetrahydro-1H-furo[3,4-c]pyrrol-5 (3H)-yl)methyl)cyclopropyl)methoxy)quinazolin-4-yl)-3,8-diazabicyclo[3.2.1]octane-8-carboxylate (16 mg) in ACN (2 mL) and 4N HCl in dioxane (0.5 ml) was stirred at RT for 0.5 h. The volatiles was removed in vacuo to give a residual, which was purified by pre-HPLC to afford the desired compound (5 mg, 38%) as a white solid. ¹H NMR (600 MHz, CD₃OD) δ 8.03-7.89 (m, 1H), 7.59 (d, J=8.3 Hz, 1H), 7.48 (m, 1H), 7.35 (d, J=8.3 Hz, 1H), 7.27 (d, J=2.5 Hz, 1H), 6.94 (d, J=2.6 Hz, 1H), 4.61 (d, J=11.6 Hz, 1H), 4.50 (d, J=11.6 Hz, 1H), 4.30 (s, 2H), 4.15 (s, 1H), 4.01 (m, 2H), 3.86 (m, 2H), 3.73-3.35 (m, 4H), 3.23-3.09 (m, 2H), 2.90 (m, 2H), 2.80 (s, 1H), 2.43 (m, 1H), 2.36-2.25 (m, 1H), 2.18 (m, 4H), 2.03 (m, 1H), 1.90-1.75 (m, 2H), 1.36-1.26 (m, 3H), 1.00 (m, 2H), 0.91 (m, 2H). MS: 636.6 (M+H⁺).

### Example 25

### 9-(4-((1R,5S)-3,8-diazabicyclo[3.2.1]octan-3-yl)-8-fluoro-2-((1-(((R)-3-methylmorpholino)methyl)cyclopropyl)methoxy)quinazolin-7-yl)-2,3-dihydro-1H-cyclopenta[a]naphthalen-7-ol

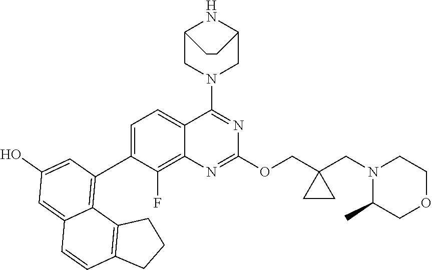 [SMILES: C[C@@H]1COCCN1CC1(COc2nc(N3CC4CCC(C3)N4)c3ccc(-c4cc(O)cc5ccc6c(c45)CCC6)c(F)c3n2)CC1]

Example 25 was synthesized following the procedure of example 24 as light yellow solid. ¹H NMR (400 MHz, MeOD) δ 8.01 (d, J=8.5 Hz, 1H), 7.60 (d, J=8.3 Hz, 1H), 7.50 (t, J=7.6 Hz, 1H), 7.36 (d, J=8.3 Hz, 1H), 7.27 (d, J=2.6 Hz, 1H), 6.95 (d, J=2.5 Hz, 1H), 4.81-4.23 (m, 5H), 4.12-3.75 (m, 6H), 3.64 (m, 1H), 3.36 (m, 1H), 3.21 (m, 1H), 2.91 (t, J=7.5 Hz, 2H), 2.64 (m, 1H), 2.44 (m, 1H), 2.33-2.24 (m, 1H), 2.16 (m, 5H), 2.03 (m, 1H), 1.84 (m, 2H), 1.36 (m, 3H), 1.13 (m, 2H), 0.99-0.87 (m, 2H). MS: 624.5 (M+H⁺).

### Example 26

### 9-(4-((1R,5S)-3,8-diazabicyclo[3.2.1]octan-3-yl)-8-fluoro-2-((1-(morpholinomethyl)cyclopropyl)methoxy)quinazolin-7-yl)-2,3-dihydro-1H-cyclopenta[a]naphthalen-7-ol

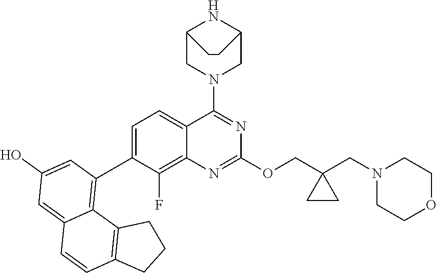 [SMILES: Oc1cc(-c2ccc3c(N4CC5CCC(C4)N5)nc(OCC4(CN5CCOCC5)CC4)nc3c2F)c2c3c(ccc2c1)CCC3]

Example 26 was synthesized following the procedure of example 24 as light yellow solid. ¹H NMR (400 MHz, MeOD) δ 8.01 (d, J=8.6 Hz, 1H), 7.59 (d, J=8.3 Hz, 1H), 7.50 (t, J=7.2 Hz, 1H), 7.35 (d, J=8.3 Hz, 1H), 7.28 (d, J=2.5 Hz, 1H), 6.96 (d, J=2.5 Hz, 1H), 4.68 (d, J=11.6 Hz, 1H), 4.50 (d, J=11.6 Hz, 1H), 4.32 (s, 2H), 4.12-4.01 (m, 4H), 3.89 (m, 2H), 3.79-3.67 (m, 2H), 3.41 (m, 1H), 3.33 (m, 1H), 3.15 (m, 2H), 2.90 (m, 2H), 2.44 (m, 1H), 2.28 (m, 1H), 2.20-2.11 (m, 4H), 1.90-1.79 (m, 2H), 1.30 (m, 1H), 1.24 (t, J=7.1 Hz, 1H), 1.04 (m, 2H), 0.95 (m, 2H). MS: 610.5 (M+H⁺).

### Example 27

### 9-(2-((1-((3-oxa-8-azabicyclo[3.2.1]octan-8-yl)methyl)cyclopropyl)methoxy)-4-((1R,5S)-3,8-diazabicyclo[3.2.1]octan-3-yl)-8-fluoroquinazolin-7-yl)-2,3-dihydro-1H-cyclopenta[a]naphthalen-7-ol

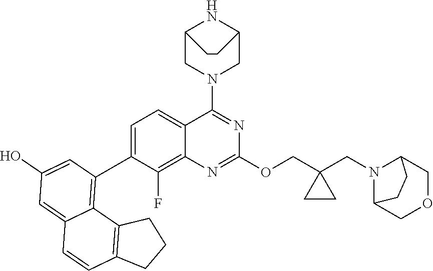 [SMILES: Oc1cc(-c2ccc3c(N4CC5CCC(C4)N5)nc(OCC4(CN5C6CCC5COC6)CC4)nc3c2F)c2c3c(ccc2c1)CCC3]

Example 27 was synthesized following the procedure of example 24 as light yellow solid. ¹H NMR (600 MHz, CD₃OD) δ 7.93 (d, J=8.6 Hz, 1H), 7.61 (d, J=8.3 Hz, 1H), 7.41 (dd, J=8.5, 6.6 Hz, 1H), 7.37 (d, J=8.3 Hz, 1H), 7.27 (d, J=2.6 Hz, 1H), 6.95 (d, J=2.6 Hz, 1H), 5.36 (m, 1H), 4.76-4.63 (m, 2H), 4.59-4.50 (m, 2H), 4.30 (m, 2H), 4.29-4.17 (m, 3H), 3.88 (m, 4H), 3.30-3.22 (m, 2H), 2.92 (m, 2H), 2.45 (m, 1H), 2.28 (m, 2H), 2.25-2.18 (m, 4H), 2.05 (m, 2H), 1.89-1.79 (m, 2H), 1.65-1.58 (m, 1H), 1.05-0.97 (m, 2H), 0.95-0.89 (m, 2H). MS: 636.5 (M+H⁺).

### Example 28

### 9-(2-((1-((3-oxa-8-azabicyclo[3.2.1]octan-8-yl)methyl)cyclopropyl)methoxy)-4-((1R,5S)-3,8-diazabicyclo[3.2.1]octan-3-yl)-6,8-difluoroquinazolin-7-yl)-2,3-dihydro-1H-cyclopenta[a]naphthalen-7-ol

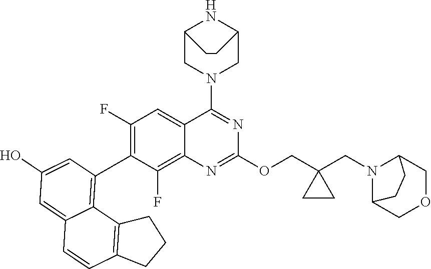 [SMILES: Oc1cc(-c2c(F)cc3c(N4CC5CCC(C4)N5)nc(OCC4(CN5C6CCC5COC6)CC4)nc3c2F)c2c3c(ccc2c1)CCC3]

Example 28 was synthesized following the procedure of example 24 as light yellow solid. ¹H NMR (400 MHz, MeOD) δ 7.66 (d, J=9.6 Hz, 1H), 7.61 (d, J=8.3 Hz, 1H), 7.36 (d, J=8.3 Hz, 1H), 7.29 (d, J=2.6 Hz, 1H), 6.96 (d, J=2.6 Hz, 1H), 5.34 (td, J=4.6, 2.3 Hz, 1H), 4.62 (d, J=14.0 Hz, 1H), 4.53-4.49 (m, 2H), 4.24 (m, 5H), 3.87 (m, 3H), 3.76 (d, J=14.1 Hz, 1H), 3.25 (d, J=7.4 Hz, 1H), 2.92 (t, J=7.5 Hz, 2H), 2.40 (t, J=7.3 Hz, 2H), 2.26 (m, 3H), 2.19 (m, 4H), 2.03 (m, 2H), 1.89-1.83 (m, 2H), 1.59 (m, 1H), 0.93-0.86 (m, 4H). MS: 654.5 (M+H⁺).

### Example 29

### 9-(4-((1R,5S)-3,8-diazabicyclo[3.2.1]octan-3-yl)-6,8-difluoro-2-((1-(morpholinomethyl)cyclopropyl)methoxy)quinazolin-7-yl)-2,3-dihydro-1H-cyclopenta[a]naphthalen-7-ol

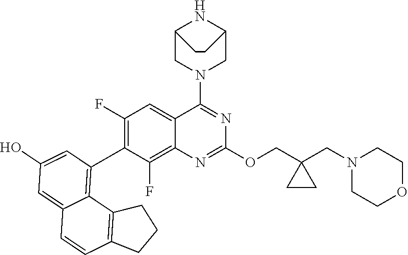 [SMILES: Oc1cc(-c2c(F)cc3c(N4CC5CCC(C4)N5)nc(OCC4(CN5CCOCC5)CC4)nc3c2F)c2c3c(ccc2c1)CCC3]

Example 29 was synthesized following the procedure of example 24 as light yellow solid. ¹H NMR (400 MHz, MeOD) δ 7.67 (d, J=9.6 Hz, 1H), 7.61 (d, J=8.3 Hz, 1H), 7.36 (d, J=8.3 Hz, 1H), 7.29 (d, J=2.6 Hz, 1H), 6.96 (d, J=2.6 Hz, 1H), 5.34 (td, J=4.6, 2.3 Hz, 1H), 4.68 (d, J=14.0 Hz, 1H), 4.60-4.55 (m, 1H), 4.47 (m, 2H), 4.27 (m, 3H), 4.06 (m, 2H), 3.89 (m, 2H), 3.80 (m, 2H), 3.65 (t, J=6.4 Hz, 1H), 3.36 (t, J=12 Hz, 2H), 3.18 (m, 2H), 2.92 (m, 3H), 2.40 (m, 2H), 2.19 (m, 3H), 1.91 (m, 3H), 1.60 (m, 1H), 0.99 (m, 2H), 0.90 (m, 2H). MS: 628.6 (M+H⁺).

### Example 30

### 9-(4-((1R,5S)-3,8-diazabicyclo[3.2.1]octan-3-yl)-2-(((2R,7aS)-2-fluorotetrahydro-1H-pyrrolizin-7a(5H)-yl)methoxy)pyrido[4,3-d]pyrimidin-7-yl)-2,3-dihydro-1H-cyclopenta[a]naphthalen-7-ol

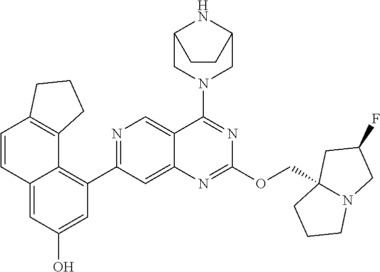 [SMILES: Oc1cc(-c2cc3nc(OC[C@@]45CCCN4C[C@H](F)C5)nc(N4CC5CCC(C4)N5)c3cn2)c2c3c(ccc2c1)CCC3]

Example 30 was synthesized following the procedure of example 2 as white solid. ¹H NMR (400 MHz, DMSO) δ 9.75 (s, 1H), 9.24 (s, 1H), 7.63-7.61 (d, J=8.0 Hz, 1H), 7.38-7.36 (m, 2H), 7.25-7.24 (d, J=2.4 Hz, 1H), 6.97-6.97 (d, J=2.4 Hz, 1H), 5.34-5.21 (m, 1H), 4.52-4.36 (m, 2H), 4.14-4.01 (m, 2H), 3.68-3.01 (m, 8H), 2.88-2.82 (m, 3H), 2.20-2.00 (m, 5H), 1.85-1.65 (m, 9H).

MS: 581.4 (M+H⁺).

### Example 31

### 9-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-2,3-dihydro-1H-cyclopenta[a]naphthalen-7-ol

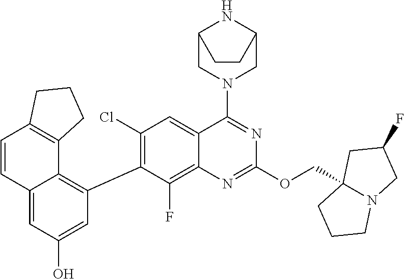 [SMILES: Oc1cc(-c2c(Cl)cc3c(N4CC5CCC(C4)N5)nc(OC[C@@]45CCCN4C[C@H](F)C5)nc3c2F)c2c3c(ccc2c1)CCC3]

Example 31 was synthesized following the procedure of example 2 as white solid.

MS: 632.4 (M+H⁺).

### Example 32: Cellular Proliferation Assay in GP2d Cells

The inhibitory effect of test compounds on cellular proliferation was determined using CellTiterGlo® luminescent cell viability assay (Promega) following manufacturer's instruction.

GP2d cells (ECACC #95090715) were cultured in DMEM medium supplemented with 10% fetal bovine serum, 10 U/ml penicillin and 10 ug/ml streptomycin. Cells were plated in 96-well plates at a density of 2000 cells/well and allowed attach for 120-24 hours. Then the cells were treated with various concentrations of test compounds for 72 hours. CellTiter-Glo® Reagent was added to wells and incubated for 10 minutes at room temperature. The plates were read on GloMax® 96 Microplate Luminometer or compatible microplate readers. The percentage of cellular viability was calculated as:

$$Cellular viability = ( mean RLU sample - mean RLU blank ) / ( RLU cell control - RLU blank ) \times 100. {IC}_{50} value was calculated using GraphPad Prism ( San Diego , CA ) . The assays were carried out in triplicates .$$

TABLE 1

| Example. No. | GP2d IC₅₀ (μM) |
|---|---|
| 1 | 0.680 |
| 2 | 0.130 |
| 3 | 8.8 |
| 4 | 4.75 |
| 5 | 2.86 |
| 6 | 9.29 |
| 7 | 0.92 |
| 8 | 0.284 |
| 9 | 0.175 |
| 10 | 0.261 |
| 11 | 0.169 |
| 12 | 0.0484 |
| 13 | 0.010 |
| 14 | 0.429 |
| 15 | 0.541 |
| 16 | 0.849 |
| 17 | 16 |
| 18 | 17 |
| 19 | 3.06 |
| 20 | 2.41 |
| 21 | 0.263 |
| 22 | 3.34 |
| 23 | 0.908 |
| 24 | 0.120 |
| 25 | 0.303 |
| 26 | 0.195 |
| 27 | 0.370 |
| 28 | 0.253 |
| 29 | 0.273 |
| 30 | 0.409 |
| 31 | 0.737 |

### Example 33: Detection of Phosphorylation of ERK1/2 in AGS Cells

The inhibitory effect of test compounds on phosphorylation of ERK1/2 were determined using the AlphaLISA® SureFire® Ultra™ p-ERK 1/2 (Thr202/Tyr204) assay following the manufacturer's instructions (ALSU-PERK-A500, PerkinElmer Inc.).

AGS cells (ATCC CRL-1739) with KRAS-G12D mutation were cultured in DMEM medium supplemented with 10% fetal bovine serum, 10 U/ml penicillin and 10 μg/ml streptomycin. Cells were plated in 96-well plates at a density of 4000 cells/well. After 16-18 hours incubation, various concentrations of compounds were added to cells with a final concentration of 0.2% DMSO. After 3 hours, the medium was removed, and the cells were lysed in 50 uL lysis buffer. 10 uL of lysate was transferred to a half area 96-well plate, 5 ul of acceptor mix was added to wells and incubated at room temperature for 1 hour, then 5 ul of donor mix was added to wells. After 1 hour incubation at room temperature, the plate was read on an Alpha Technology-compatible plate reader, using standard AlphaLISA settings. The assays were carried out in duplicates.

TABLE 2

| Example. No. | IC₅₀ (μM) |
|---|---|
| 1 | 0.408 |
| 2 | 0.078 |

### Example 34: p-ERK Assay

The expression of p-ERK was detected in this assay. At first, the AGS cells (40000 cells/well) were seeded in 96-well plate culture overnight and treated with KRAS-G12D compounds inhibitors at concentrations of 1, 0.33, 0.11, 0.03, 0.01, 0.004, 0.001, 0.0004, 0.0001 μM for 3 h at 37° C. Then, add an equal volume (100 μL) of 8% paraformaldehyde solution to fix and crosslink the cells to the microplate for 15 minutes. Add 100 μL of 1× permeabilization solution to wells for 30 min and add 100 μL of 1× blocking solution for 2 h. Wash with PBS 3 times after each reaction. At last, Incubation with primary antibody (Anti-mouse β-Actin and Anti-rabbit p-ERK) and secondary antibody (Goat anti-Mouse IgG H&L IRDye 800CW and Goat anti-Rabbit IgG H&L IRDye 680RW) for 2 h, As appropriate, image the microplate with an IR scanner or develop the HRP labeled microplate and read it with a spectrophotometer. The fluorescence signal was monitored using a microplate reader (Azure biosystems, Sapphire biomolecular imager) using excitation and emission wavelengths of 680 and 800 nm, respectively. Export data. The inhibitor dose-reponse curves were analyzed using normalized IC50 regression curve fitting with control based normalization.

TABLE 3

| Example. No. | IC₅₀ (μM) |
|---|---|
| 1 | 0.107 |
| 2 | 0.011 |
| 3 | 8.8 |
| 4 | 4.9 |
| 5 | 0.037 |
| 6 | 0.588 |
| 7 | 0.149 |
| 8 | 0.041 |
| 9 | 0.031 |
| 10 | 0.205 |
| 11 | 0.135 |
| 12 | 0.017 |
| 13 | 0.022 |
| 14 | 0.085 |
| 15 | 0.159 |
| 16 | 0.005 |
| 17 | 0.272 |
| 18 | 0.24 |
| 19 | NA |
| 20 | 0.226 |
| 21 | 0.052 |
| 22 | NA |
| 23 | 0.033 |
| 24 | 0.024 |
| 25 | 0.018 |
| 26 | 0.039 |
| 27 | 0.131 |
| 28 | 0.087 |
| 29 | 0.044 |

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof.

All patents, patent applications, and publications cited herein are fully incorporated by reference herein in their entirety.

1.-43. (canceled)

44. A compound of Formula Ia: 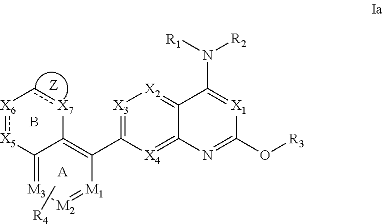
wherein:
X₁, X₂, X₃ and X₄ independently represent C or N, and when X₁, X₂, X₃ or X₄ represents C, it is optionally substituted with C₁-C₄ alkyl, halogen, hydroxyl or CN;
R₁ represents 5-membered or 6-membered heterocyclyl or heteroaryl group containing 1, 2 or 3 N atoms, and R₂ represents H or C₁-C₄ alkyl, wherein R₁ is optionally substituted with one or more of C₁-C₄ alkyl, amino, halogen, or —C₁-C₄ alkyl-CN; or
R₁ and R₂ taken together with the nitrogen atom to which they are attached form a N-containing heterocyclyl, wherein the N-containing heterocyclyl is optionally substituted with one or more of C₁-C₄ alkyl, amino, halogen, hydroxyl or —C₁-C₄ alkyl-CN;
R³ is selected from a group consisting of (C₃-C₆ cycloalkyl)C₁-C₄ alkyl, (heterocyclo)C₁-C₄ alkyl, (aryl)C₁-C₄ alkyl, and (hetereoaryl)C₁-C₄ alkyl, wherein the cycloalkyl, heterocyclo, aryl and hetereoaryl are optionally substituted with one or more of halogen, C₁-C₄ alkyl, —N(C₁-C₄ alkyl)₂, heterocyclyl, C₁-C₄ alkoxy, (hydroxy)C₁-C₄ alkyl, (amino)C₁-C₄ alkyl, —C₁-C₄ alkyl-O—C(═O)—N(C₁-C₄ alkyl)₂, —C₁-C₄ alkyl-NH—C(═O)—N(C₁-C₄ alkyl)₂, —O—C(═O)-heterocyclyl, —C₁-C₄ alkyl-N(C₁-C₄ alkyl)₂, or —C₁-C₄ alkyl-heterocyclyl;
M₁, M₂ and M₃ independently represent C or N, and when M₁, M₂ or M₃ represents C, it is optionally substituted with R₄, wherein R₄ represents hydroxyl, halogen, C₁-C₄ alkyl or amino;
X₅, X₆ and X₇ independently represent C or N;
each [symbol] in ring B independently represents a single or double bond; and
\textcircled{Z} represents a 5-7 membered carbocyclic, 5-7 membered aryl or 5-7-membered heterocyclyl or hetereoaryl fused with ring B having 1-3 heteroatoms selected from the group consisting of N, S and O, wherein the carbocyclic, aryl, heterocyclyl or hetereoaryl is optionally substituted with one or more of halogen, C₁-C₄ alkyl, oxo, (hydroxyl)C₁-C₄ alkyl or hydroxyl;
or a pharmaceutically acceptable salt or solvate thereof.

45. The compound of claim 44, wherein
X₁ represents N, X₂ represents C, X₃ represents N, and X₄ represents C;
X₁ represents N, X₂ represents C, X₃ represents C, and X₄ represents C;
X₁ represents C, X₂ represents C, X₃ represents N, and X₄ represents C; or
X₁ represents N, X₂ represents C, X₃ represents C, and X₄ represents N.

46. The compound of claim 44, wherein M₁, M₂ and M₃ each represent C; and M₂ is optionally substituted with R₄.

47. The compound of claim 44, wherein R₁ is selected from the group consisting of: 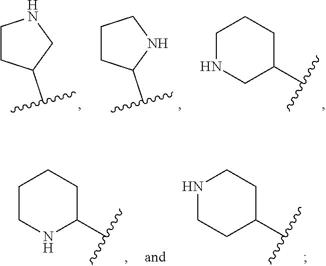 [SMILES: *C1CCNC1 ; *C1CCCN1 ; *C1CCCNC1 ; *C1CCCCN1 ; *C1CCNCC1]
and
wherein R₁ is optionally substituted with one or more of C₁-C₄ alkyl, amino, halogen, or —C₁-C₄ alkyl-CN

48. The compound of claim 47, wherein R₂ is H.

49. The compound of claim 44, wherein R₁ and R₂ taken together with the nitrogen atom to which they are attached form a N-containing heterocyclyl selected from the group consisting of: 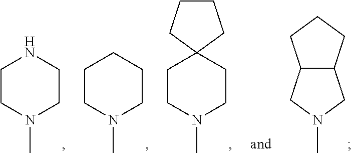
and
wherein the N-containing heterocyclyl is optionally substituted with one or more of C₁-C₄ alkyl, amino, halogen, hydroxyl or —C₁-C₄ alkyl-CN.

50. The compound of claim 44, wherein R₁ and R₂ taken together with the nitrogen atom to which they are attached form a N-containing heterocyclyl selected from the group consisting of: 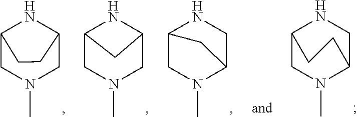
and
wherein the N-containing heterocyclyl is optionally substituted with one or more of C₁-C₄ alkyl, amino, halogen, hydroxyl or —C₁-C₄ alkyl-CN.

51. The compound of claim 44, wherein R₃ is (C₃-C₆ cycloalkyl)C₁-C₄ alkyl.

52. The compound of claim 44, wherein R₃ is (heterocyclo)C₁-C₄ alkyl and the heterocyclo is selected from the group consisting of:

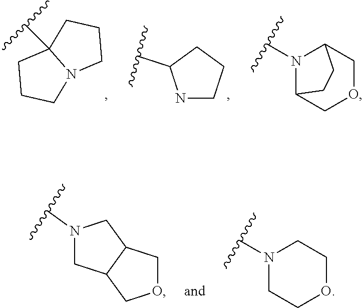

53. The compound of claim 44, wherein R₃ is selected from the group consisting of:

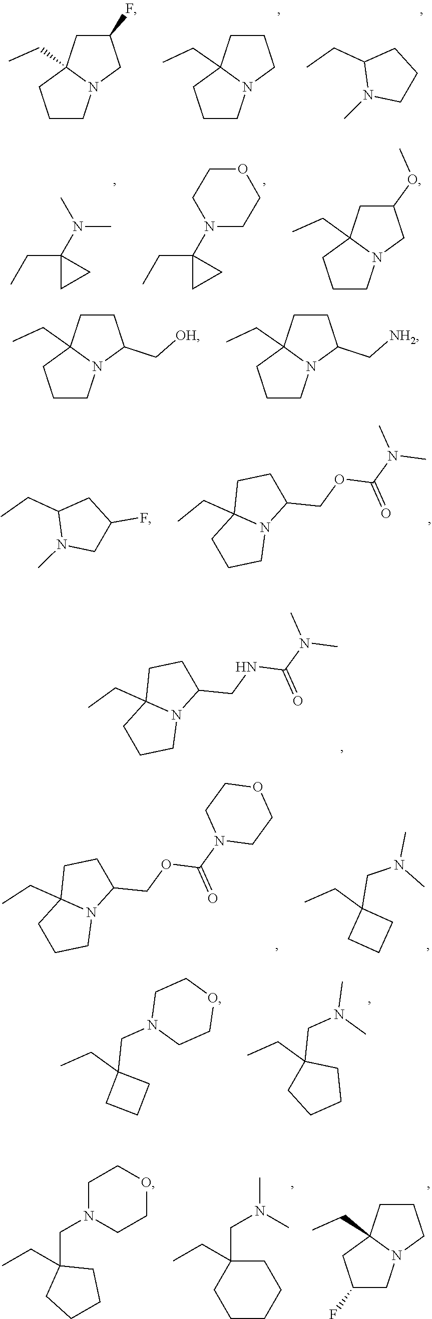

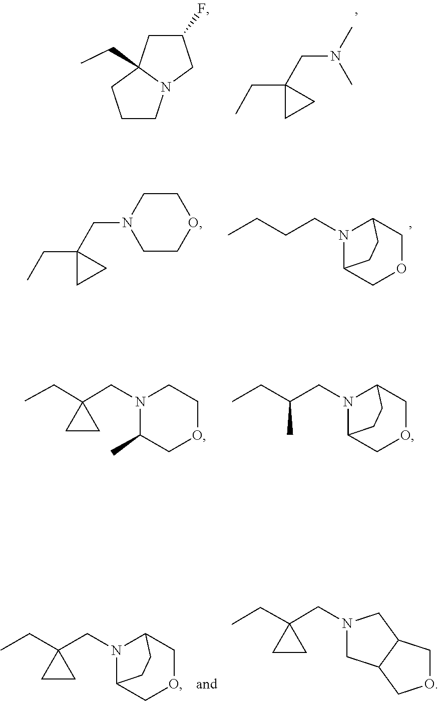

54. The compound of claim 44, wherein R⁴ is selected from the group consisting of: hydroxyl and amino.

55. The compound of claim 44, wherein \textcircled{Z} is optionally substituted with methyl or F.

56. The compound of claim 44, wherein \textcircled{Z} represents a fused 5 membered carbocyclic, 5 membered aryl or 5-membered heterocyclyl or hetereoaryl having 1-3 heteroatoms selected from the group consisting of N, S and O; and
wherein the carbocyclic, aryl, heterocyclyl or hetereoaryl is optionally substituted with one or more of halogen, C₁-C₄ alkyl, or hydroxyl.

57. The compound of claim 44 wherein \textcircled{Z} represents a fused 6 membered carbocyclic, 6 membered aryl or 6-membered heterocyclyl or hetereoaryl having 1-3 heteroatoms selected from the group consisting of N, S and O; and
wherein the carbocyclic, aryl, heterocyclyl or hetereoaryl is optionally substituted with one or more of halogen, C₁-C₄ alkyl, or hydroxyl.

58. The compound of claim 44, wherein each [symbol] in ring B represents a double bond.

59. A pharmaceutical composition comprising the compound claim 44, or a pharmaceutically acceptable salt or solvate thereof, and a pharmaceutically acceptable carrier.

60. A compound selected from the group consisting of: 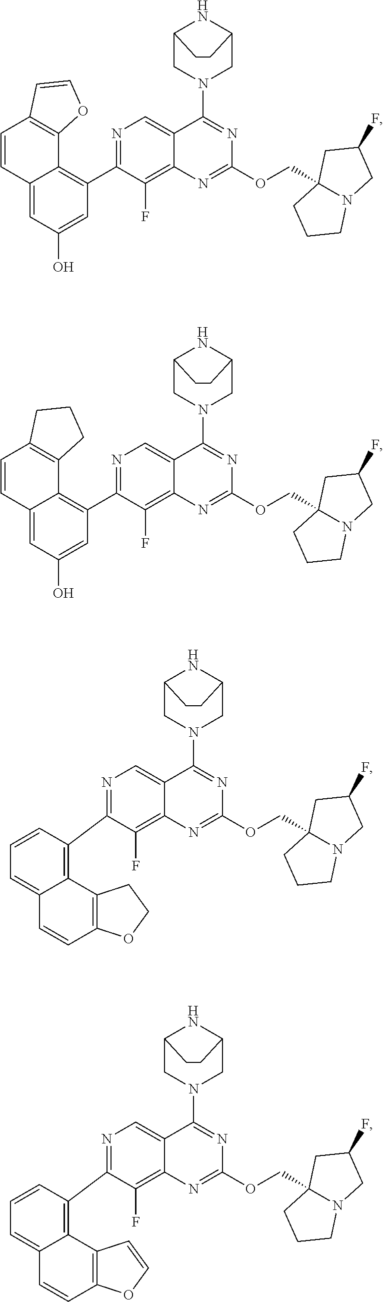 [SMILES: Oc1cc(-c2ncc3c(N4CC5CCC(C4)N5)nc(OC[C@@]45CCCN4C[C@H](F)C5)nc3c2F)c2c(ccc3ccoc32)c1 ; Oc1cc(-c2ncc3c(N4CC5CCC(C4)N5)nc(OC[C@@]45CCCN4C[C@H](F)C5)nc3c2F)c2c3c(ccc2c1)CCC3 ; Fc1c(-c2cccc3ccc4c(c23)CCO4)ncc2c(N3CC4CCC(C3)N4)nc(OC[C@@]34CCCN3C[C@H](F)C4)nc12 ; Fc1c(-c2cccc3ccc4occc4c23)ncc2c(N3CC4CCC(C3)N4)nc(OC[C@@]34CCCN3C[C@H](F)C4)nc12] 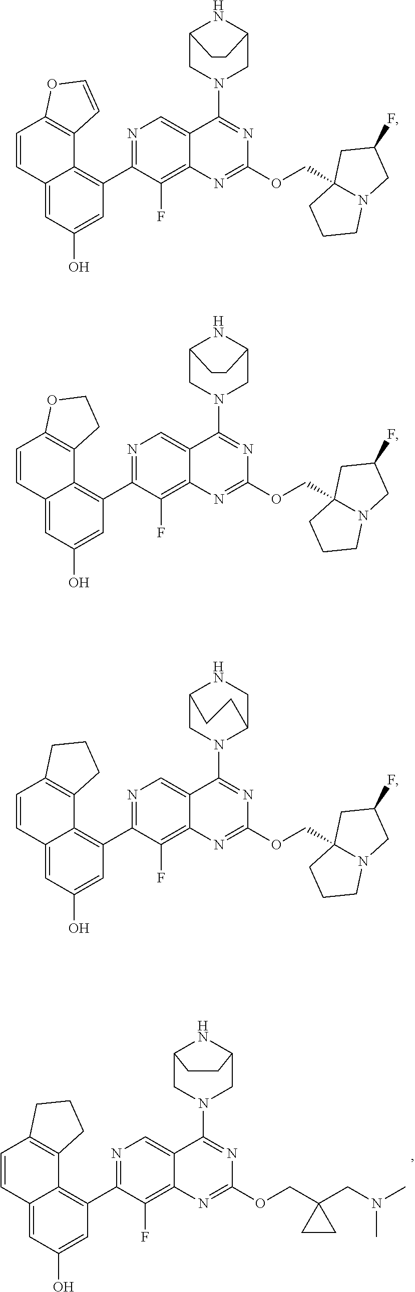 [SMILES: Oc1cc(-c2ncc3c(N4CC5CCC(C4)N5)nc(OC[C@@]45CCCN4C[C@H](F)C5)nc3c2F)c2c(ccc3occc32)c1 ; Oc1cc(-c2ncc3c(N4CC5CCC(C4)N5)nc(OC[C@@]45CCCN4C[C@H](F)C5)nc3c2F)c2c3c(ccc2c1)OCC3 ; Oc1cc(-c2ncc3c(N4CC5CCC4CN5)nc(OC[C@@]45CCCN4C[C@H](F)C5)nc3c2F)c2c3c(ccc2c1)CCC3 ; CN(C)CC1(COc2nc(N3CC4CCC(C3)N4)c3cnc(-c4cc(O)cc5ccc6c(c45)CCC6)c(F)c3n2)CC1] 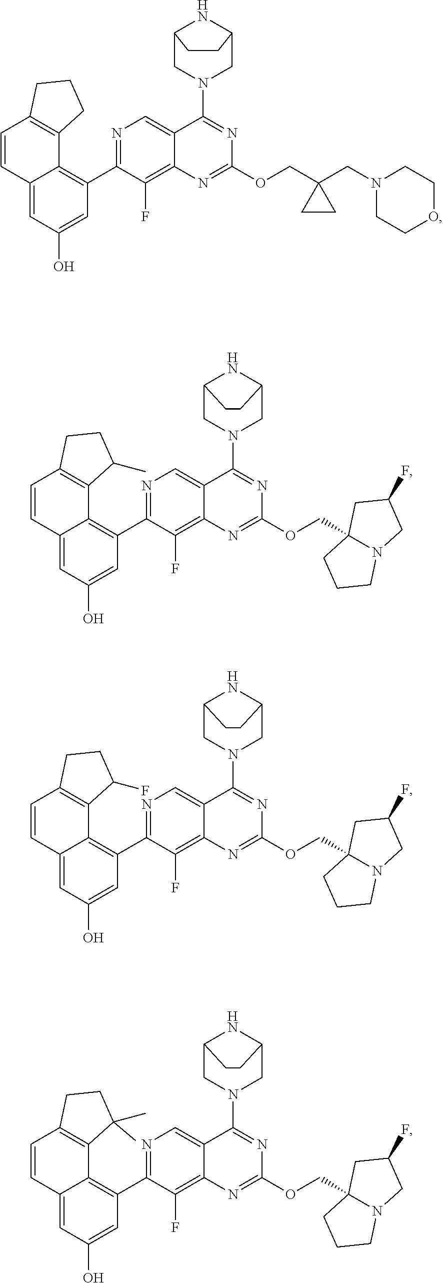 [SMILES: Oc1cc(-c2ncc3c(N4CC5CCC(C4)N5)nc(OCC4(CN5CCOCC5)CC4)nc3c2F)c2c3c(ccc2c1)CCC3 ; CC1CCc2ccc3cc(O)cc(-c4ncc5c(N6CC7CCC(C6)N7)nc(OC[C@@]67CCCN6C[C@H](F)C7)nc5c4F)c3c21 ; Oc1cc(-c2ncc3c(N4CC5CCC(C4)N5)nc(OC[C@@]45CCCN4C[C@H](F)C5)nc3c2F)c2c3c(ccc2c1)CCC3F ; CC1(C)CCc2ccc3cc(O)cc(-c4ncc5c(N6CC7CCC(C6)N7)nc(OC[C@@]67CCCN6C[C@H](F)C7)nc5c4F)c3c21] 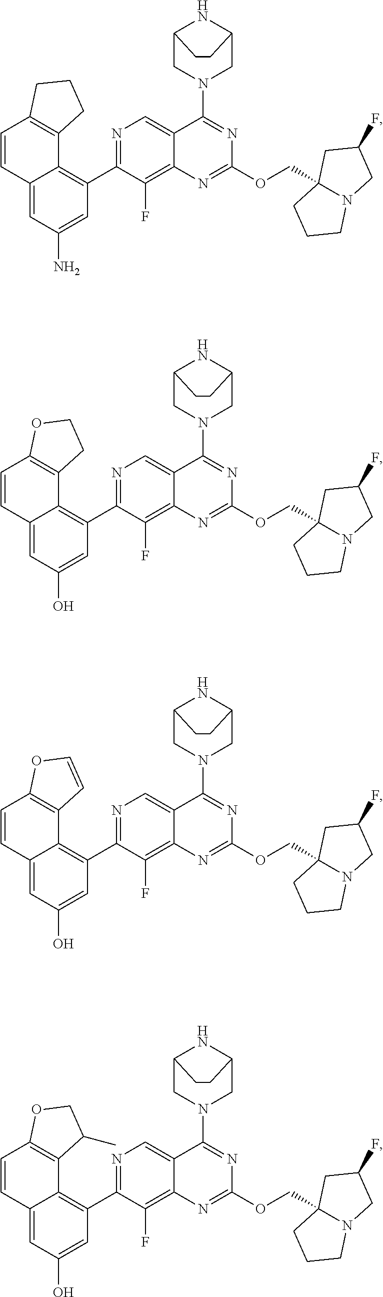 [SMILES: Nc1cc(-c2ncc3c(N4CC5CCC(C4)N5)nc(OC[C@@]45CCCN4C[C@H](F)C5)nc3c2F)c2c3c(ccc2c1)CCC3 ; Oc1cc(-c2ncc3c(N4CC5CCC(C4)N5)nc(OC[C@@]45CCCN4C[C@H](F)C5)nc3c2F)c2c3c(ccc2c1)OCC3 ; Oc1cc(-c2ncc3c(N4CC5CCC(C4)N5)nc(OC[C@@]45CCCN4C[C@H](F)C5)nc3c2F)c2c(ccc3occc32)c1 ; CC1COc2ccc3cc(O)cc(-c4ncc5c(N6CC7CCC(C6)N7)nc(OC[C@@]67CCCN6C[C@H](F)C7)nc5c4F)c3c21] 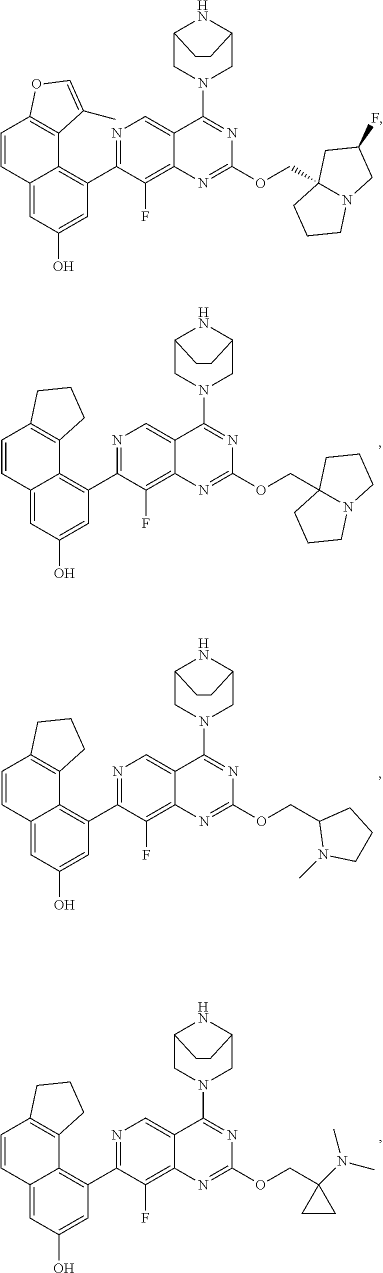 [SMILES: Cc1coc2ccc3cc(O)cc(-c4ncc5c(N6CC7CCC(C6)N7)nc(OC[C@@]67CCCN6C[C@H](F)C7)nc5c4F)c3c12 ; Oc1cc(-c2ncc3c(N4CC5CCC(C4)N5)nc(OCC45CCCN4CCC5)nc3c2F)c2c3c(ccc2c1)CCC3 ; CN1CCCC1COc1nc(N2CC3CCC(C2)N3)c2cnc(-c3cc(O)cc4ccc5c(c34)CCC5)c(F)c2n1 ; CN(C)C1(COc2nc(N3CC4CCC(C3)N4)c3cnc(-c4cc(O)cc5ccc6c(c45)CCC6)c(F)c3n2)CC1] 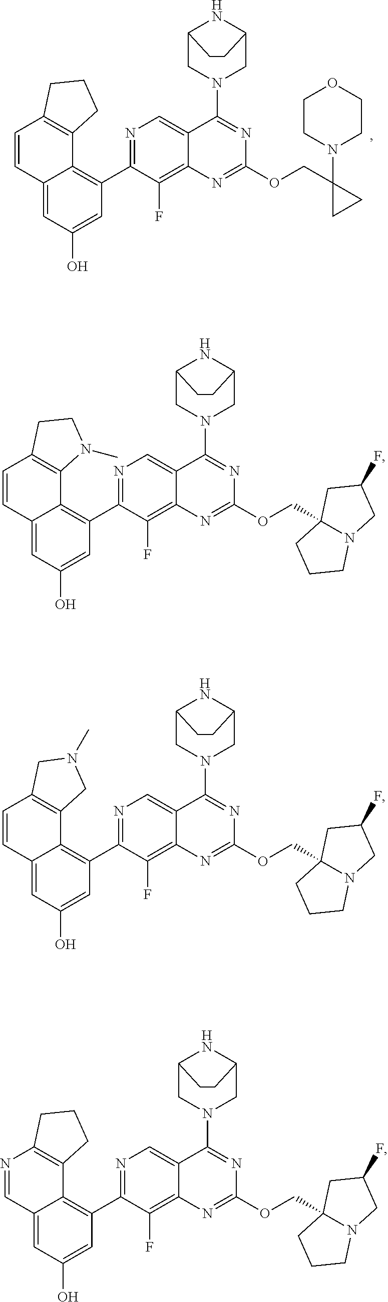 [SMILES: Oc1cc(-c2ncc3c(N4CC5CCC(C4)N5)nc(OCC4(N5CCOCC5)CC4)nc3c2F)c2c3c(ccc2c1)CCC3 ; CN1CCc2ccc3cc(O)cc(-c4ncc5c(N6CC7CCC(C6)N7)nc(OC[C@@]67CCCN6C[C@H](F)C7)nc5c4F)c3c21 ; CN1Cc2ccc3cc(O)cc(-c4ncc5c(N6CC7CCC(C6)N7)nc(OC[C@@]67CCCN6C[C@H](F)C7)nc5c4F)c3c2C1 ; Oc1cc(-c2ncc3c(N4CC5CCC(C4)N5)nc(OC[C@@]45CCCN4C[C@H](F)C5)nc3c2F)c2c3c(ncc2c1)CCC3] 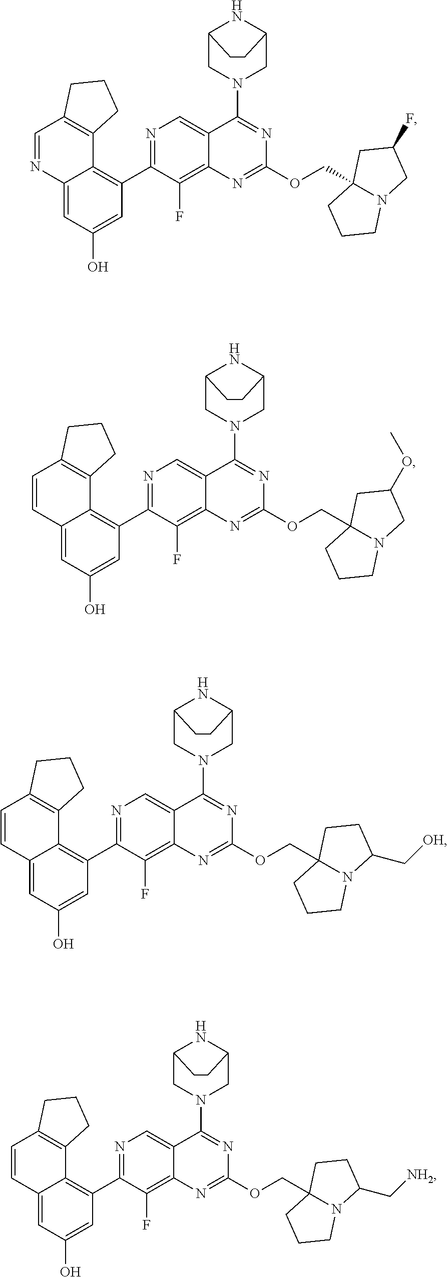 [SMILES: Oc1cc(-c2ncc3c(N4CC5CCC(C4)N5)nc(OC[C@@]45CCCN4C[C@H](F)C5)nc3c2F)c2c3c(cnc2c1)CCC3 ; COC1CN2CCCC2(COc2nc(N3CC4CCC(C3)N4)c3cnc(-c4cc(O)cc5ccc6c(c45)CCC6)c(F)c3n2)C1 ; OCC1CCC2(COc3nc(N4CC5CCC(C4)N5)c4cnc(-c5cc(O)cc6ccc7c(c56)CCC7)c(F)c4n3)CCCN12 ; NCC1CCC2(COc3nc(N4CC5CCC(C4)N5)c4cnc(-c5cc(O)cc6ccc7c(c56)CCC7)c(F)c4n3)CCCN12] 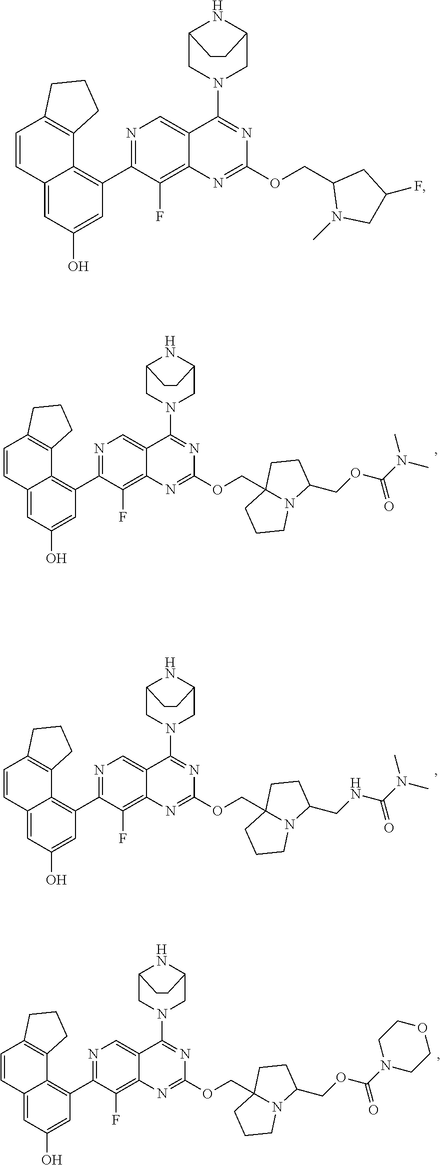 [SMILES: CN1CC(F)CC1COc1nc(N2CC3CCC(C2)N3)c2cnc(-c3cc(O)cc4ccc5c(c34)CCC5)c(F)c2n1 ; CN(C)C(=O)OCC1CCC2(COc3nc(N4CC5CCC(C4)N5)c4cnc(-c5cc(O)cc6ccc7c(c56)CCC7)c(F)c4n3)CCCN12 ; CN(C)C(=O)NCC1CCC2(COc3nc(N4CC5CCC(C4)N5)c4cnc(-c5cc(O)cc6ccc7c(c56)CCC7)c(F)c4n3)CCCN12 ; O=C(OCC1CCC2(COc3nc(N4CC5CCC(C4)N5)c4cnc(-c5cc(O)cc6ccc7c(c56)CCC7)c(F)c4n3)CCCN12)N1CCOCC1] 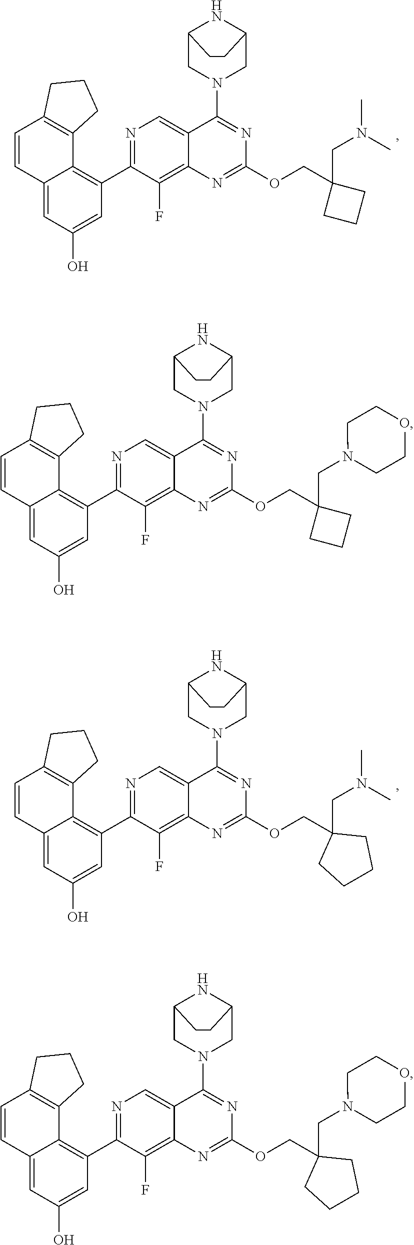 [SMILES: CN(C)CC1(COc2nc(N3CC4CCC(C3)N4)c3cnc(-c4cc(O)cc5ccc6c(c45)CCC6)c(F)c3n2)CCC1 ; Oc1cc(-c2ncc3c(N4CC5CCC(C4)N5)nc(OCC4(CN5CCOCC5)CCC4)nc3c2F)c2c3c(ccc2c1)CCC3 ; CN(C)CC1(COc2nc(N3CC4CCC(C3)N4)c3cnc(-c4cc(O)cc5ccc6c(c45)CCC6)c(F)c3n2)CCCC1 ; Oc1cc(-c2ncc3c(N4CC5CCC(C4)N5)nc(OCC4(CN5CCOCC5)CCCC4)nc3c2F)c2c3c(ccc2c1)CCC3] 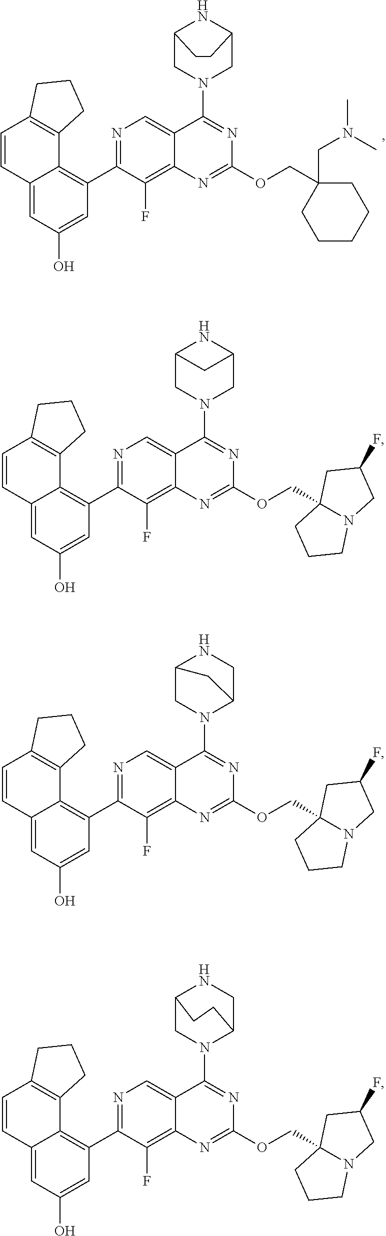 [SMILES: CN(C)CC1(COc2nc(N3CC4CCC(C3)N4)c3cnc(-c4cc(O)cc5ccc6c(c45)CCC6)c(F)c3n2)CCCCC1 ; Oc1cc(-c2ncc3c(N4CC5CC(C4)N5)nc(OC[C@@]45CCCN4C[C@H](F)C5)nc3c2F)c2c3c(ccc2c1)CCC3 ; Oc1cc(-c2ncc3c(N4CC5CC4CN5)nc(OC[C@@]45CCCN4C[C@H](F)C5)nc3c2F)c2c3c(ccc2c1)CCC3 ; Oc1cc(-c2ncc3c(N4CC5CCC4CN5)nc(OC[C@@]45CCCN4C[C@H](F)C5)nc3c2F)c2c3c(ccc2c1)CCC3] 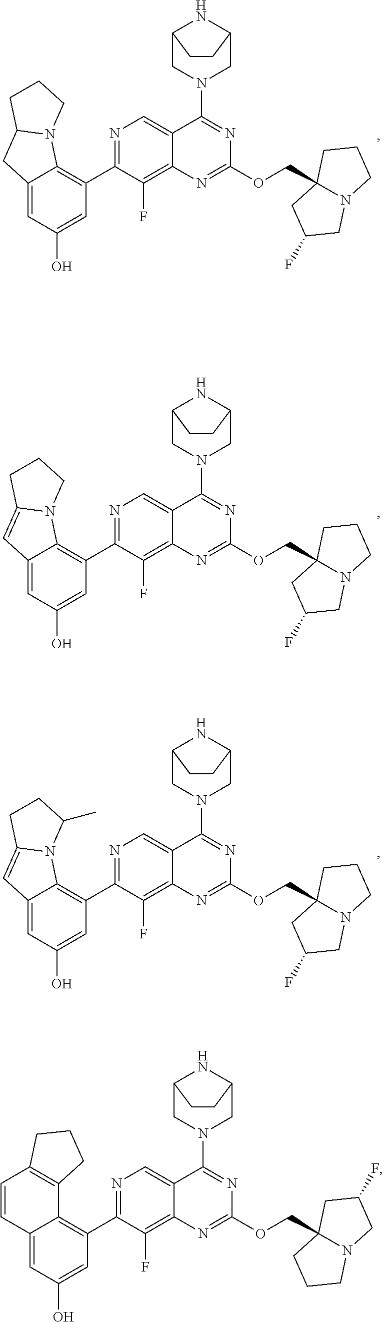 [SMILES: Oc1cc2c(c(-c3ncc4c(N5CC6CCC(C5)N6)nc(OC[C@@]56CCCN5C[C@H](F)C6)nc4c3F)c1)N1CCCC1C2 ; Oc1cc(-c2ncc3c(N4CC5CCC(C4)N5)nc(OC[C@@]45CCCN4C[C@H](F)C5)nc3c2F)c2c(c1)cc1n2CCC1 ; CC1CCc2cc3cc(O)cc(-c4ncc5c(N6CC7CCC(C6)N7)nc(OC[C@@]67CCCN6C[C@H](F)C7)nc5c4F)c3n21 ; Oc1cc(-c2ncc3c(N4CC5CCC(C4)N5)nc(OC[C@]45CCCN4C[C@@H](F)C5)nc3c2F)c2c3c(ccc2c1)CCC3] 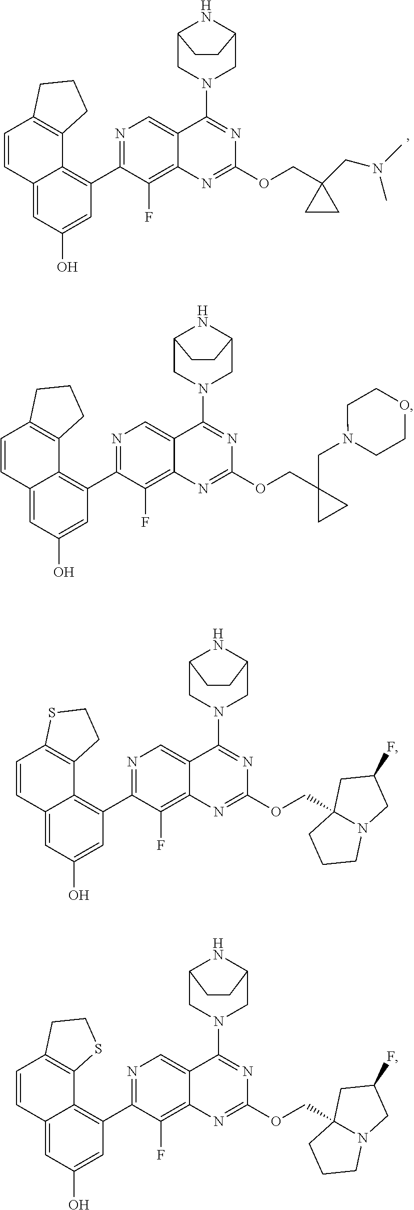 [SMILES: CN(C)CC1(COc2nc(N3CC4CCC(C3)N4)c3cnc(-c4cc(O)cc5ccc6c(c45)CCC6)c(F)c3n2)CC1 ; Oc1cc(-c2ncc3c(N4CC5CCC(C4)N5)nc(OCC4(CN5CCOCC5)CC4)nc3c2F)c2c3c(ccc2c1)CCC3 ; Oc1cc(-c2ncc3c(N4CC5CCC(C4)N5)nc(OC[C@@]45CCCN4C[C@H](F)C5)nc3c2F)c2c3c(ccc2c1)SCC3 ; Oc1cc(-c2ncc3c(N4CC5CCC(C4)N5)nc(OC[C@@]45CCCN4C[C@H](F)C5)nc3c2F)c2c3c(ccc2c1)CCS3] 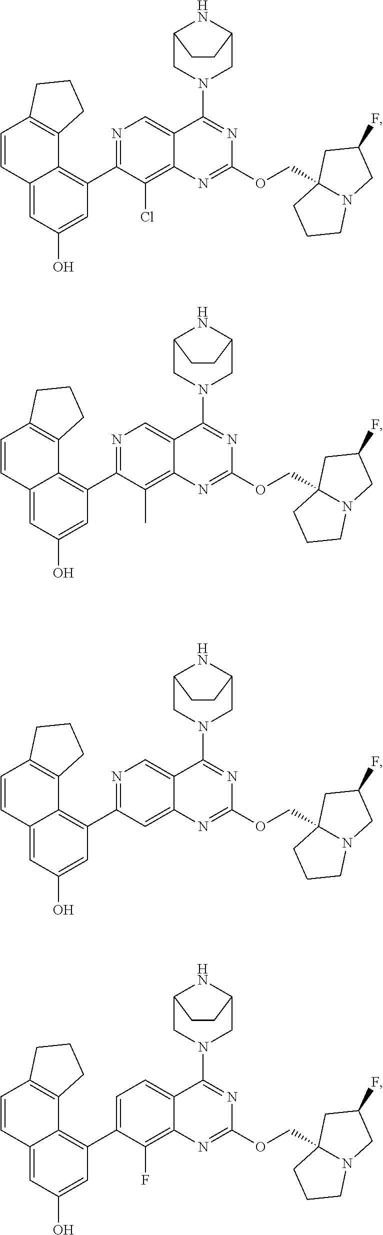 [SMILES: Oc1cc(-c2ncc3c(N4CC5CCC(C4)N5)nc(OC[C@@]45CCCN4C[C@H](F)C5)nc3c2Cl)c2c3c(ccc2c1)CCC3 ; Cc1c(-c2cc(O)cc3ccc4c(c23)CCC4)ncc2c(N3CC4CCC(C3)N4)nc(OC[C@@]34CCCN3C[C@H](F)C4)nc12 ; Oc1cc(-c2cc3nc(OC[C@@]45CCCN4C[C@H](F)C5)nc(N4CC5CCC(C4)N5)c3cn2)c2c3c(ccc2c1)CCC3 ; Oc1cc(-c2ccc3c(N4CC5CCC(C4)N5)nc(OC[C@@]45CCCN4C[C@H](F)C5)nc3c2F)c2c3c(ccc2c1)CCC3] 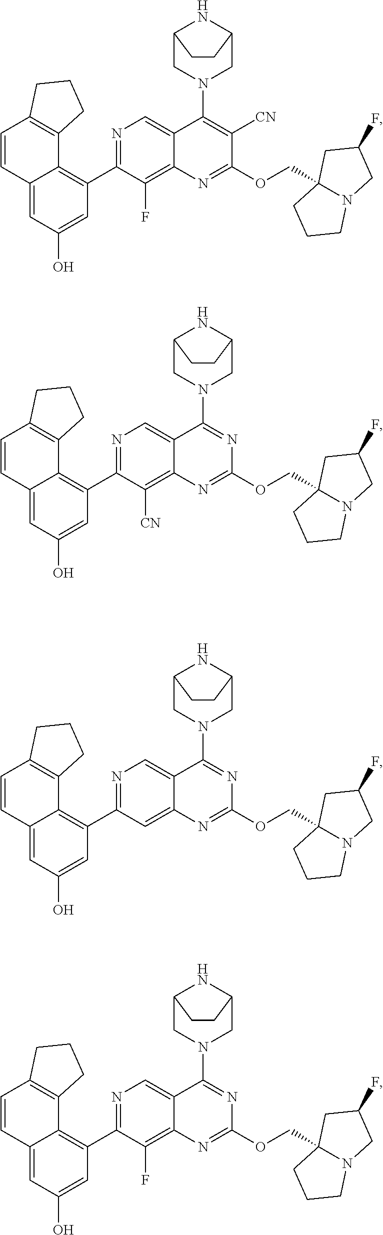 [SMILES: N#Cc1c(OC[C@@]23CCCN2C[C@H](F)C3)nc2c(F)c(-c3cc(O)cc4ccc5c(c34)CCC5)ncc2c1N1CC2CCC(C1)N2 ; N#Cc1c(-c2cc(O)cc3ccc4c(c23)CCC4)ncc2c(N3CC4CCC(C3)N4)nc(OC[C@@]34CCCN3C[C@H](F)C4)nc12 ; Oc1cc(-c2cc3nc(OC[C@@]45CCCN4C[C@H](F)C5)nc(N4CC5CCC(C4)N5)c3cn2)c2c3c(ccc2c1)CCC3 ; Oc1cc(-c2ncc3c(N4CC5CCC(C4)N5)nc(OC[C@@]45CCCN4C[C@H](F)C5)nc3c2F)c2c3c(ccc2c1)CCC3] 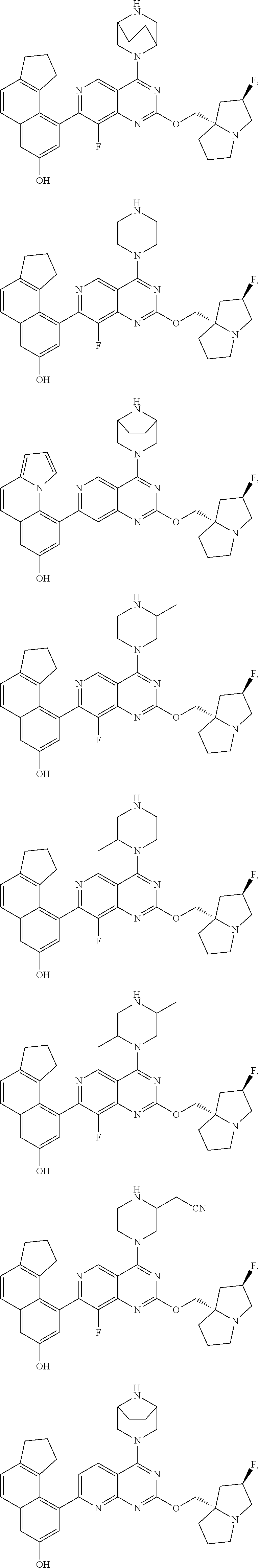 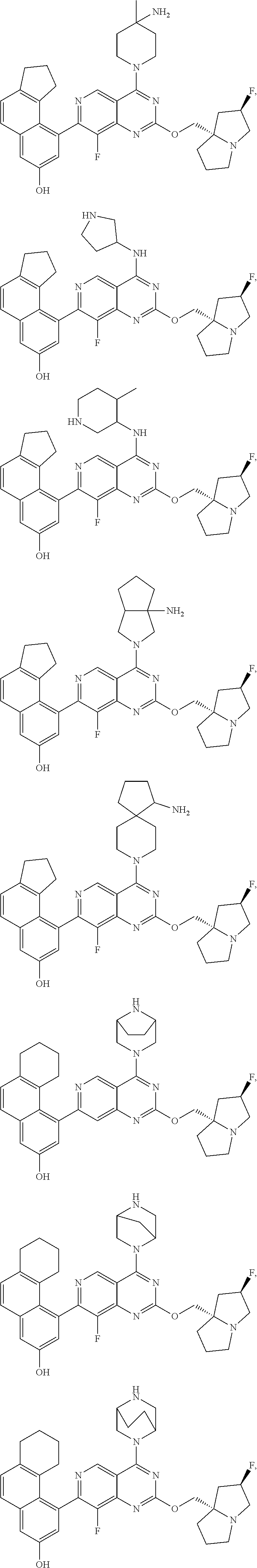 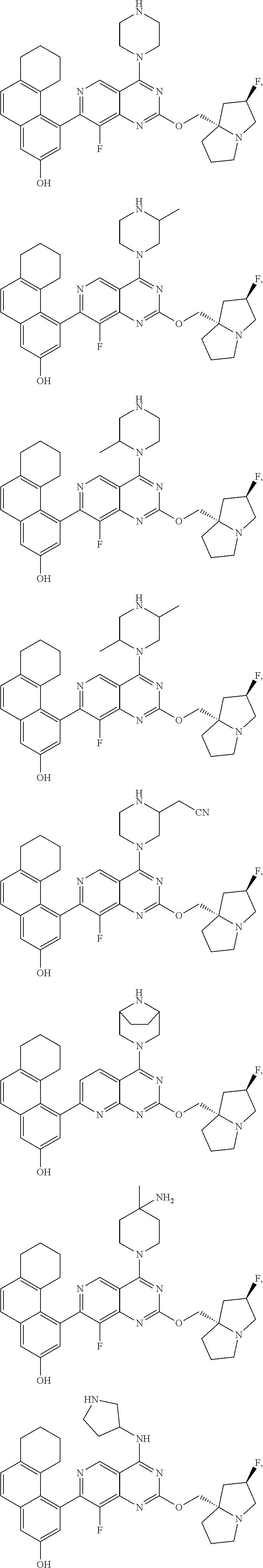 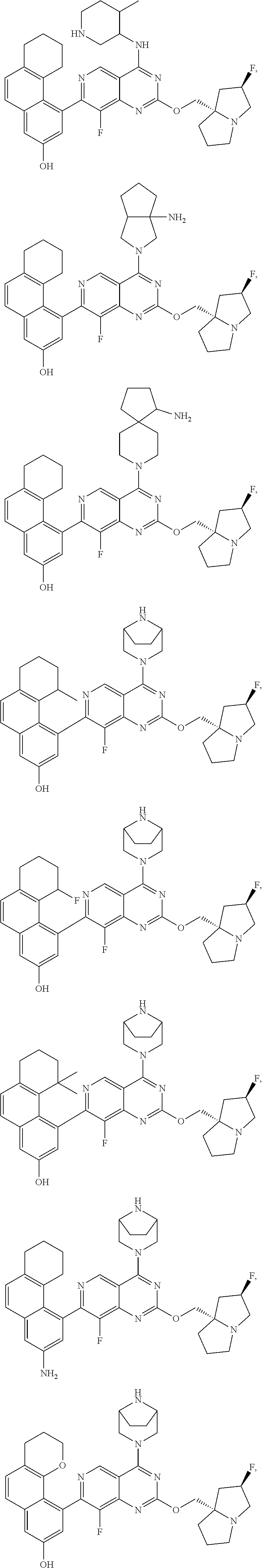 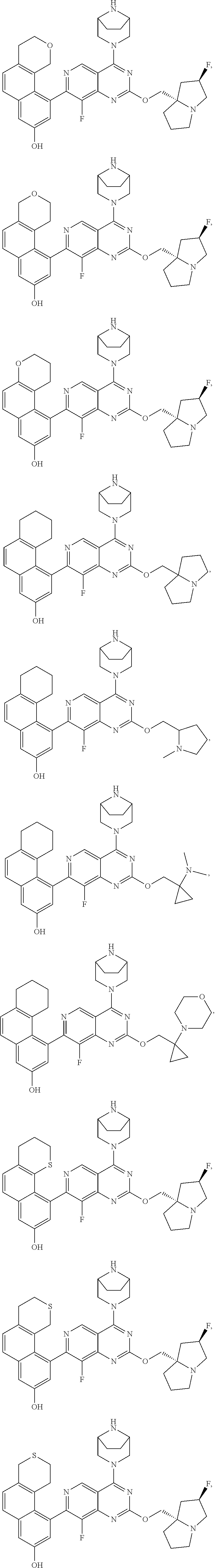 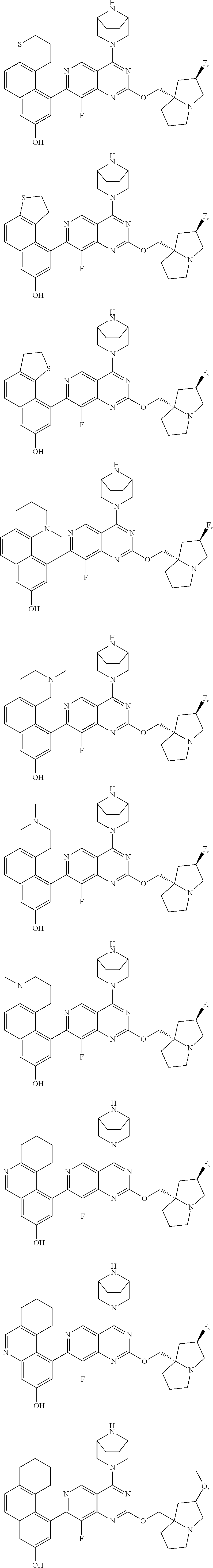 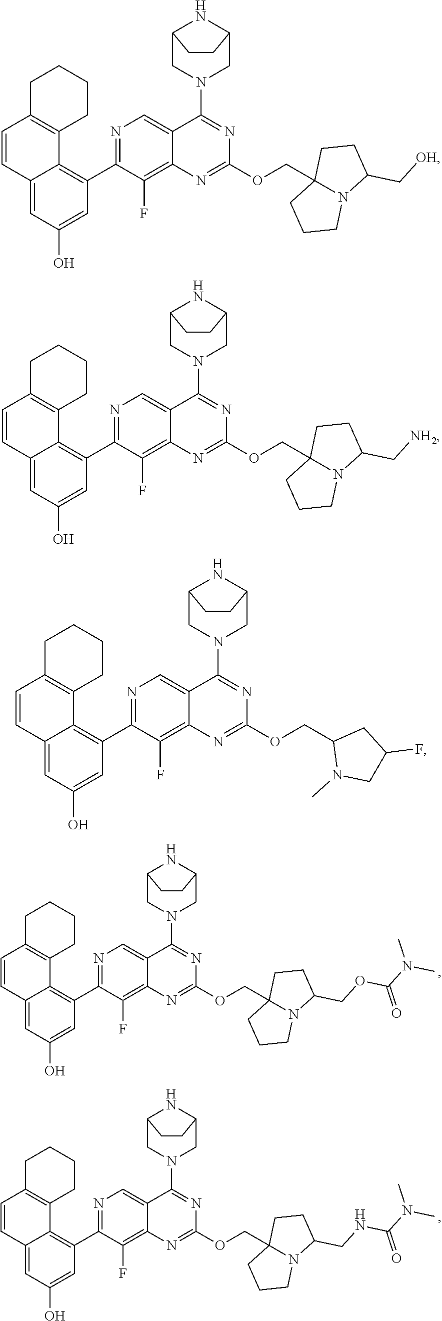 [SMILES: OCC1CCC2(COc3nc(N4CC5CCC(C4)N5)c4cnc(-c5cc(O)cc6ccc7c(c56)CCCC7)c(F)c4n3)CCCN12 ; NCC1CCC2(COc3nc(N4CC5CCC(C4)N5)c4cnc(-c5cc(O)cc6ccc7c(c56)CCCC7)c(F)c4n3)CCCN12 ; CN1CC(F)CC1COc1nc(N2CC3CCC(C2)N3)c2cnc(-c3cc(O)cc4ccc5c(c34)CCCC5)c(F)c2n1 ; CN(C)C(=O)OCC1CCC2(COc3nc(N4CC5CCC(C4)N5)c4cnc(-c5cc(O)cc6ccc7c(c56)CCCC7)c(F)c4n3)CCCN12 ; CN(C)C(=O)NCC1CCC2(COc3nc(N4CC5CCC(C4)N5)c4cnc(-c5cc(O)cc6ccc7c(c56)CCCC7)c(F)c4n3)CCCN12] 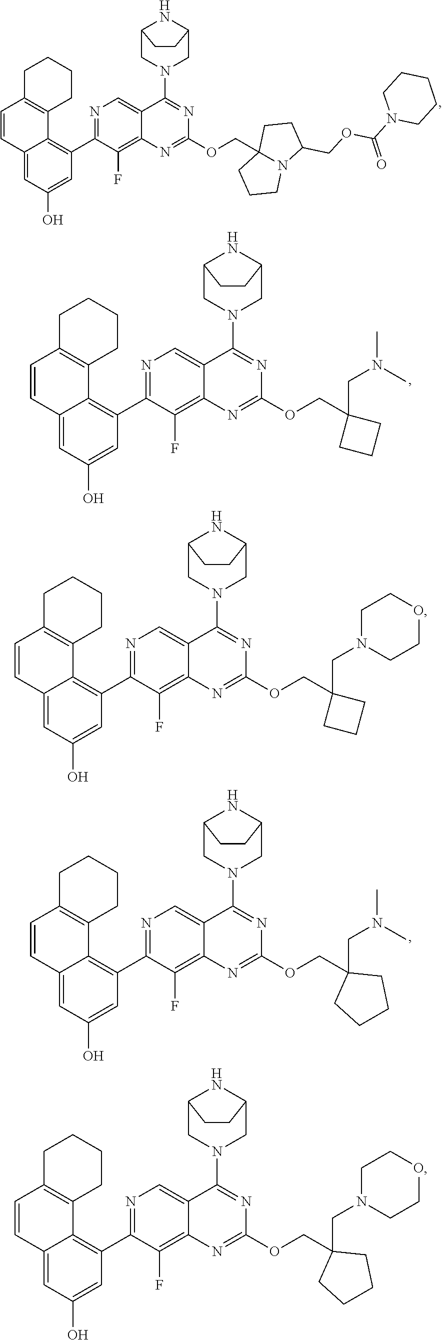 [SMILES: O=C(OCC1CCC2(COc3nc(N4CC5CCC(C4)N5)c4cnc(-c5cc(O)cc6ccc7c(c56)CCCC7)c(F)c4n3)CCCN12)N1CCCCC1 ; CN(C)CC1(COc2nc(N3CC4CCC(C3)N4)c3cnc(-c4cc(O)cc5ccc6c(c45)CCCC6)c(F)c3n2)CCC1 ; Oc1cc(-c2ncc3c(N4CC5CCC(C4)N5)nc(OCC4(CN5CCOCC5)CCC4)nc3c2F)c2c3c(ccc2c1)CCCC3 ; CN(C)CC1(COc2nc(N3CC4CCC(C3)N4)c3cnc(-c4cc(O)cc5ccc6c(c45)CCCC6)c(F)c3n2)CCCC1 ; Oc1cc(-c2ncc3c(N4CC5CCC(C4)N5)nc(OCC4(CN5CCOCC5)CCCC4)nc3c2F)c2c3c(ccc2c1)CCCC3] 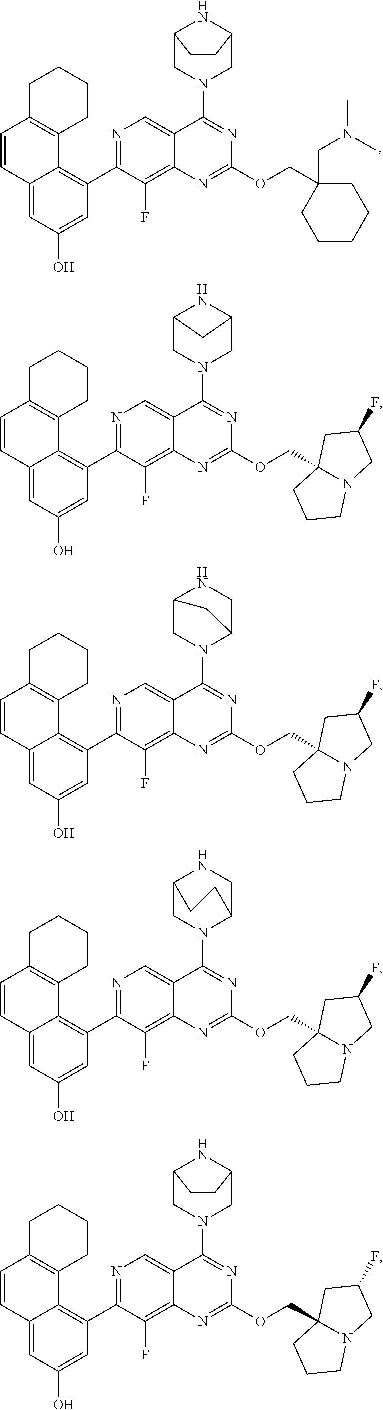 [SMILES: CN(C)CC1(COc2nc(N3CC4CCC(C3)N4)c3cnc(-c4cc(O)cc5ccc6c(c45)CCCC6)c(F)c3n2)CCCCC1 ; Oc1cc(-c2ncc3c(N4CC5CC(C4)N5)nc(OC[C@@]45CCCN4C[C@H](F)C5)nc3c2F)c2c3c(ccc2c1)CCCC3 ; Oc1cc(-c2ncc3c(N4CC5CC4CN5)nc(OC[C@@]45CCCN4C[C@H](F)C5)nc3c2F)c2c3c(ccc2c1)CCCC3 ; Oc1cc(-c2ncc3c(N4CC5CCC4CN5)nc(OC[C@@]45CCCN4C[C@H](F)C5)nc3c2F)c2c3c(ccc2c1)CCCC3 ; Oc1cc(-c2ncc3c(N4CC5CCC(C4)N5)nc(OC[C@]45CCCN4C[C@@H](F)C5)nc3c2F)c2c3c(ccc2c1)CCCC3] 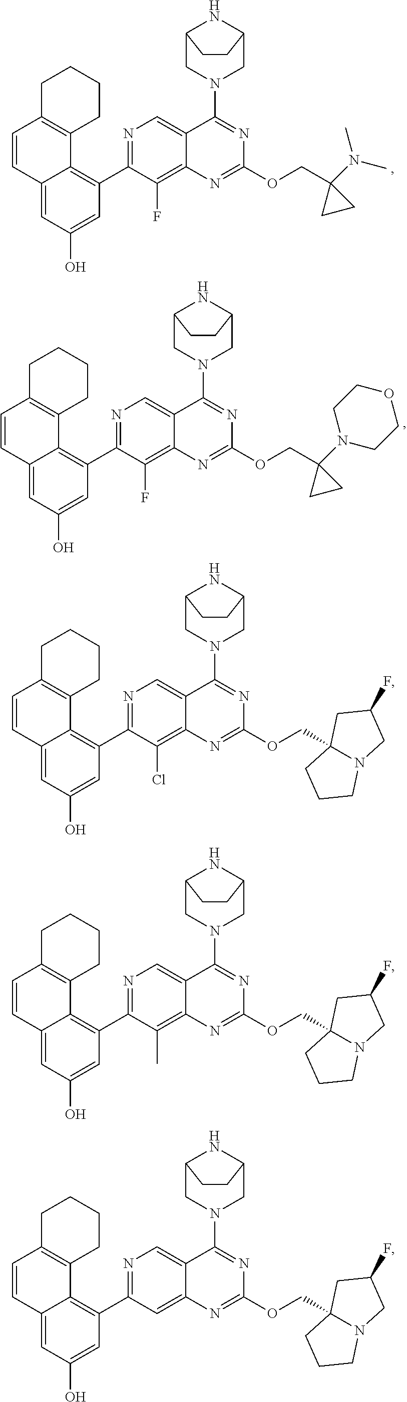 [SMILES: CN(C)C1(COc2nc(N3CC4CCC(C3)N4)c3cnc(-c4cc(O)cc5ccc6c(c45)CCCC6)c(F)c3n2)CC1 ; Oc1cc(-c2ncc3c(N4CC5CCC(C4)N5)nc(OCC4(N5CCOCC5)CC4)nc3c2F)c2c3c(ccc2c1)CCCC3 ; Oc1cc(-c2ncc3c(N4CC5CCC(C4)N5)nc(OC[C@@]45CCCN4C[C@H](F)C5)nc3c2Cl)c2c3c(ccc2c1)CCCC3 ; Cc1c(-c2cc(O)cc3ccc4c(c23)CCCC4)ncc2c(N3CC4CCC(C3)N4)nc(OC[C@@]34CCCN3C[C@H](F)C4)nc12 ; Oc1cc(-c2cc3nc(OC[C@@]45CCCN4C[C@H](F)C5)nc(N4CC5CCC(C4)N5)c3cn2)c2c3c(ccc2c1)CCCC3] 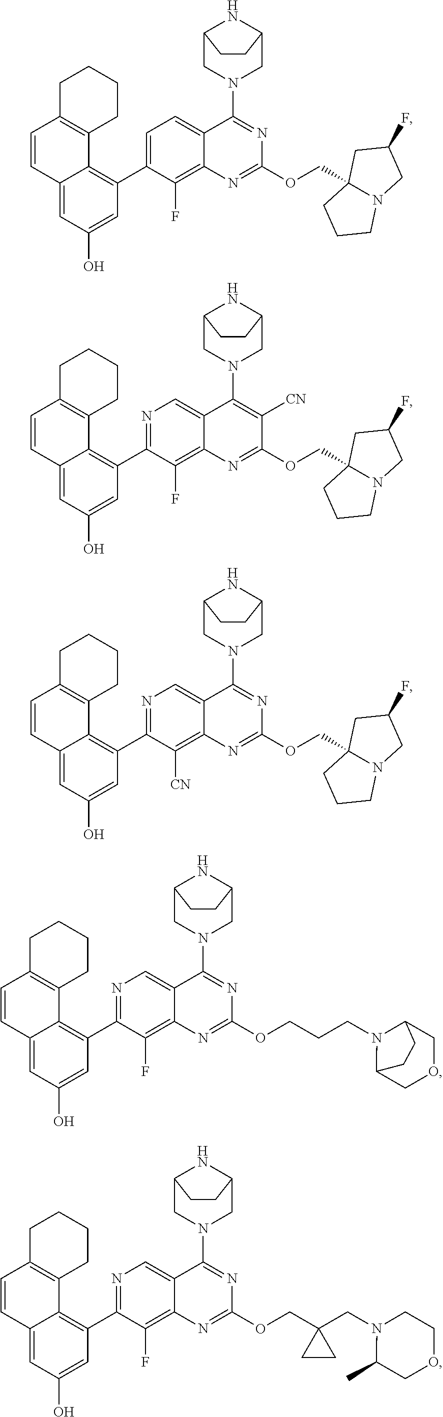 [SMILES: Oc1cc(-c2ccc3c(N4CC5CCC(C4)N5)nc(OC[C@@]45CCCN4C[C@H](F)C5)nc3c2F)c2c3c(ccc2c1)CCCC3 ; N#Cc1c(OC[C@@]23CCCN2C[C@H](F)C3)nc2c(F)c(-c3cc(O)cc4ccc5c(c34)CCCC5)ncc2c1N1CC2CCC(C1)N2 ; N#Cc1c(-c2cc(O)cc3ccc4c(c23)CCCC4)ncc2c(N3CC4CCC(C3)N4)nc(OC[C@@]34CCCN3C[C@H](F)C4)nc12 ; Oc1cc(-c2ncc3c(N4CC5CCC(C4)N5)nc(OCCCN4C5CCC4COC5)nc3c2F)c2c3c(ccc2c1)CCCC3 ; C[C@@H]1COCCN1CC1(COc2nc(N3CC4CCC(C3)N4)c3cnc(-c4cc(O)cc5ccc6c(c45)CCCC6)c(F)c3n2)CC1] 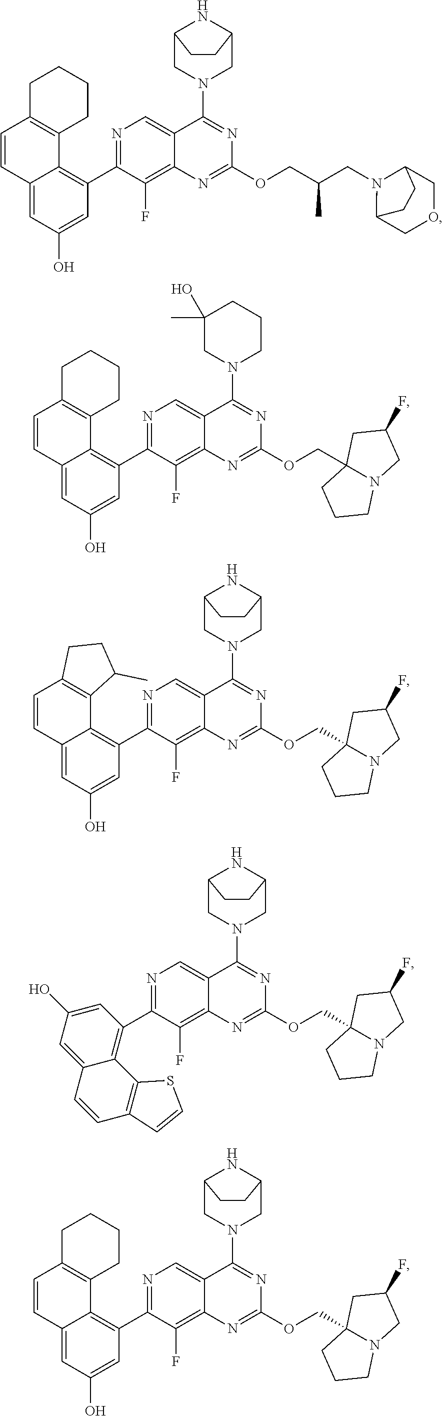 [SMILES: C[C@@H](COc1nc(N2CC3CCC(C2)N3)c2cnc(-c3cc(O)cc4ccc5c(c34)CCCC5)c(F)c2n1)CN1C2CCC1COC2 ; CC1(O)CCCN(c2nc(OCC34CCCN3C[C@H](F)C4)nc3c(F)c(-c4cc(O)cc5ccc6c(c45)CCCC6)ncc23)C1 ; CC1CCc2ccc3cc(O)cc(-c4ncc5c(N6CC7CCC(C6)N7)nc(OC[C@@]67CCCN6C[C@H](F)C7)nc5c4F)c3c21 ; Oc1cc(-c2ncc3c(N4CC5CCC(C4)N5)nc(OC[C@@]45CCCN4C[C@H](F)C5)nc3c2F)c2c(ccc3ccsc32)c1 ; Oc1cc(-c2ncc3c(N4CC5CCC(C4)N5)nc(OC[C@@]45CCCN4C[C@H](F)C5)nc3c2F)c2c3c(ccc2c1)CCCC3] 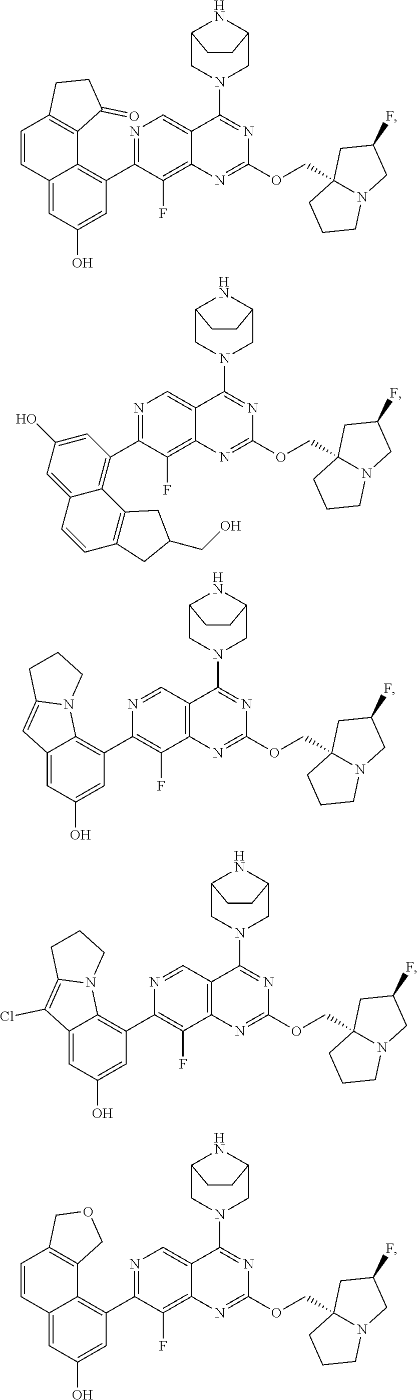 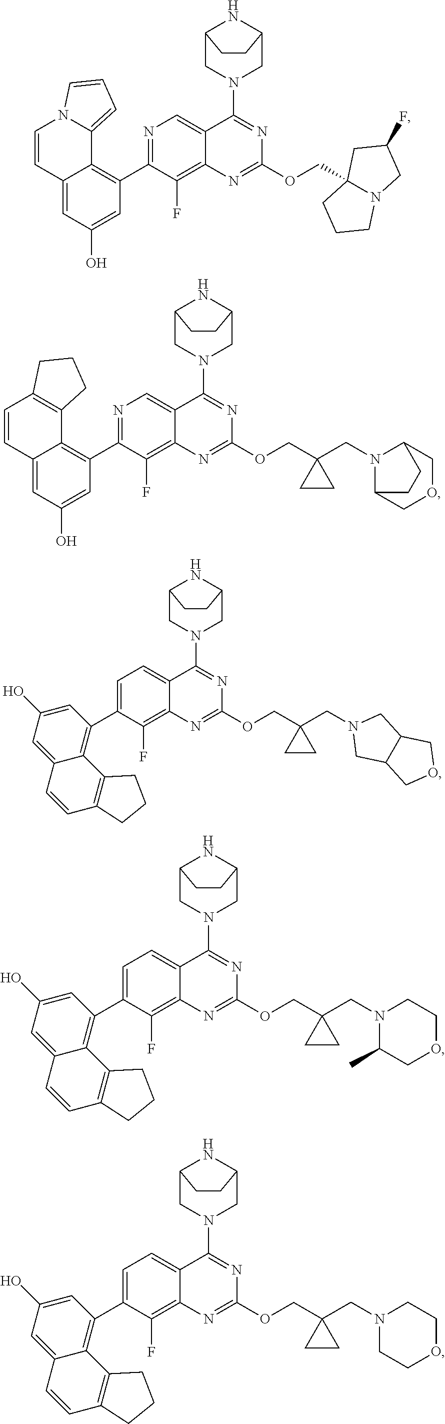 [SMILES: Oc1cc(-c2ncc3c(N4CC5CCC(C4)N5)nc(OC[C@@]45CCCN4C[C@H](F)C5)nc3c2F)c2c(ccn3cccc23)c1 ; Oc1cc(-c2ncc3c(N4CC5CCC(C4)N5)nc(OCC4(CN5C6CCC5COC6)CC4)nc3c2F)c2c3c(ccc2c1)CCC3 ; Oc1cc(-c2ccc3c(N4CC5CCC(C4)N5)nc(OCC4(CN5CC6COCC6C5)CC4)nc3c2F)c2c3c(ccc2c1)CCC3 ; C[C@@H]1COCCN1CC1(COc2nc(N3CC4CCC(C3)N4)c3ccc(-c4cc(O)cc5ccc6c(c45)CCC6)c(F)c3n2)CC1 ; Oc1cc(-c2ccc3c(N4CC5CCC(C4)N5)nc(OCC4(CN5CCOCC5)CC4)nc3c2F)c2c3c(ccc2c1)CCC3] 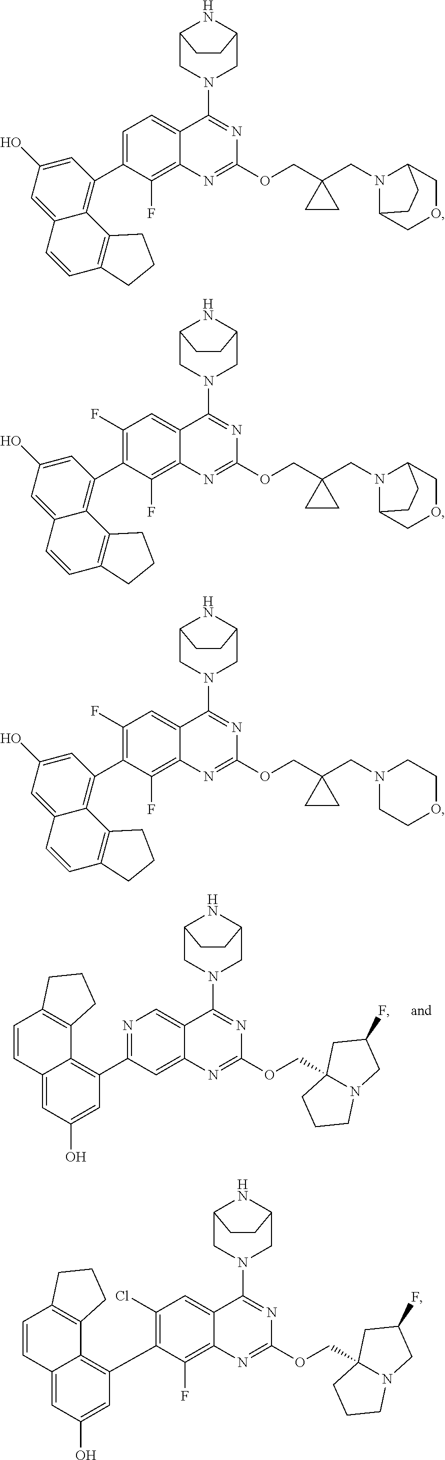 [SMILES: Oc1cc(-c2ccc3c(N4CC5CCC(C4)N5)nc(OCC4(CN5C6CCC5COC6)CC4)nc3c2F)c2c3c(ccc2c1)CCC3 ; Oc1cc(-c2c(F)cc3c(N4CC5CCC(C4)N5)nc(OCC4(CN5C6CCC5COC6)CC4)nc3c2F)c2c3c(ccc2c1)CCC3 ; Oc1cc(-c2c(F)cc3c(N4CC5CCC(C4)N5)nc(OCC4(CN5CCOCC5)CC4)nc3c2F)c2c3c(ccc2c1)CCC3 ; Oc1cc(-c2cc3nc(OC[C@@]45CCCN4C[C@H](F)C5)nc(N4CC5CCC(C4)N5)c3cn2)c2c3c(ccc2c1)CCC3 ; Oc1cc(-c2c(Cl)cc3c(N4CC5CCC(C4)N5)nc(OC[C@@]45CCCN4C[C@H](F)C5)nc3c2F)c2c3c(ccc2c1)CCC3]
or a salt or solvate thereof.

61. A pharmaceutical composition comprising the compound of claim 3, or a pharmaceutically acceptable salt or solvate thereof, and a pharmaceutically acceptable carrier.

62. A method for inhibiting KRAS G12D activity in a cell, comprising contacting the cell in which inhibition of KRAS G12D activity is desired with an effective amount of a compound of Formula Ia: 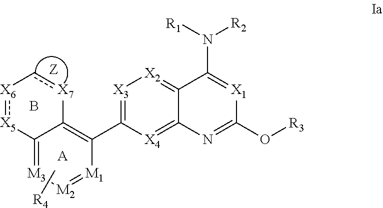
wherein:
X₁, X₂, X₃ and X₄ independently represent C or N, and when X₁, X₂, X₃ or X₄ represents C, it is optionally substituted with C₁-C₄ alkyl, halogen, hydroxyl or CN;
R₁ represents 5-membered or 6-membered heterocyclyl or heteroaryl group containing 1, 2 or 3 N atoms, and R₂ represents H or C₁-C₄ alkyl, wherein R₁ is optionally substituted with one or more of C₁-C₄ alkyl, amino, halogen, or —C₁-C₄ alkyl-CN; or
R₁ and R₂ taken together with the nitrogen atom to which they are attached form a N-containing heterocyclyl, wherein the N-containing heterocyclyl is optionally substituted with one or more of C₁-C₄ alkyl, amino, halogen, hydroxyl or —C₁-C₄ alkyl-CN;
R³ is selected from a group consisting of (C₃-C₆ cycloalkyl)C₁-C₄ alkyl, (heterocyclo)C₁-C₄ alkyl, (aryl)C₁-C₄ alkyl, and (hetereoaryl)C₁-C₄ alkyl, wherein the cycloalkyl, heterocyclo, aryl and hetereoaryl are optionally substituted with one or more of halogen, C₁-C₄ alkyl, —N(C₁-C₄ alkyl)₂, heterocyclyl, C₁-C₄ alkoxy, (hydroxy)C₁-C₄ alkyl, (amino)C₁-C₄ alkyl, —C₁-C₄ alkyl-O—C(═O)—N(C₁-C₄ alkyl)₂, —C₁-C₄ alkyl-NH—C(═O)—N(C₁-C₄ alkyl)₂, —O—C(═O)-heterocyclyl, —C₁-C₄ alkyl-N(C₁-C₄ alkyl)₂, or —C₁-C₄ alkyl-heterocyclyl;
M₁, M₂ and M₃ independently represent C or N, and when M₁, M₂ or M₃ represents C, it is optionally substituted with R₄, wherein R₄ represents hydroxyl, halogen, C₁-C₄ alkyl or amino;
X₅, X₆ and X₇ independently represent C or N;
each [symbol] in ring B independently represents a single or double bond; and
\textcircled{Z} represents a 5-7 membered carbocyclic, 5-7 membered aryl or 5-7-membered heterocyclyl or hetereoaryl fused with ring B having 1-3 heteroatoms selected from the group consisting of N, S and O, wherein the carbocyclic, aryl, heterocyclyl or hetereoaryl is optionally substituted with one or more of halogen, C₁-C₄ alkyl, oxo, (hydroxyl)C₁-C₄ alkyl or hydroxyl;
or a pharmaceutically acceptable salt or solvate thereof.

63. A method for treating a KRAS G12D-associated cancer comprising administering to a patient in need thereof a therapeutically effective amount of a compound of Formula Ia: 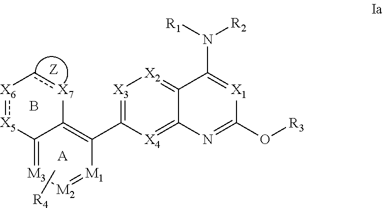
wherein:
X₁, X₂, X₃ and X₄ independently represent C or N, and when X₁, X₂, X₃ or X₄ represents C, it is optionally substituted with C₁-C₄ alkyl, halogen, hydroxyl or CN;
R₁ represents 5-membered or 6-membered heterocyclyl or heteroaryl group containing 1, 2 or 3 N atoms, and R₂ represents H or C₁-C₄ alkyl, wherein R₁ is optionally substituted with one or more of C₁-C₄ alkyl, amino, halogen, or —C₁-C₄ alkyl-CN; or
R₁ and R₂ taken together with the nitrogen atom to which they are attached form a N-containing heterocyclyl, wherein the N-containing heterocyclyl is optionally substituted with one or more of C₁-C₄ alkyl, amino, halogen, hydroxyl or —C₁-C₄ alkyl-CN;
R³ is selected from a group consisting of (C₃-C₆ cycloalkyl)C₁-C₄ alkyl, (heterocyclo)C₁-C₄ alkyl, (aryl)C₁-C₄ alkyl, and (hetereoaryl)C₁-C₄ alkyl, wherein the cycloalkyl, heterocyclo, aryl and hetereoaryl are optionally substituted with one or more of halogen, C₁-C₄ alkyl, —N(C₁-C₄ alkyl)₂, heterocyclyl, C₁-C₄ alkoxy, (hydroxy)C₁-C₄ alkyl, (amino)C₁-C₄ alkyl, —C₁-C₄ alkyl-O—C(═O)—N(C₁-C₄ alkyl)₂, —C₁-C₄ alkyl-NH—C(═O)—N(C₁-C₄ alkyl)₂, —O—C(═O)-heterocyclyl, —C₁-C₄ alkyl-N(C₁-C₄ alkyl)₂, or —C₁-C₄ alkyl-heterocyclyl;
M₁, M₂ and M₃ independently represent C or N, and when M₁, M₂ or M₃ represents C, it is optionally substituted with R₄, wherein R₄ represents hydroxyl, halogen, C₁-C₄ alkyl or amino;
X₅, X₆ and X₇ independently represent C or N;
each [symbol] in ring B independently represents a single or double bond; and
\textcircled{Z} represents a 5-7 membered carbocyclic, 5-7 membered aryl or 5-7-membered heterocyclyl or hetereoaryl fused with ring B having 1-3 heteroatoms selected from the group consisting of N, S and O, wherein the carbocyclic, aryl, heterocyclyl or hetereoaryl is optionally substituted with one or more of halogen, C₁-C₄ alkyl, oxo, (hydroxyl)C₁-C₄ alkyl or hydroxyl;
or a pharmaceutically acceptable salt or solvate thereof.

64. The method of claim 63, wherein the KRAS G12D-associated cancer is selected from one of the following groups:
a) Cardiac cancer consisting of: sarcoma including angiosarcoma, fibrosarcoma, rhabdomyosarcoma, and liposarcoma, myxoma, rhabdomyoma, fibroma, lipoma and teratoma;
b) Lung cancer consisting of: bronchogenic carcinoma including squamous cell, undifferentiated small cell, undifferentiated large cell, and adenocarcinoma, alveolar carcinoma, bronchiolar carcinoma, bronchial adenoma, sarcoma, lymphoma, chondromatous hamartoma, mesothelioma;
c) Gastrointestinal cancer consisting of: esophagus cancer including squamous cell carcinoma, adenocarcinoma, leiomyosarcoma, and lymphoma, stomach cancer including carcinoma, lymphoma, and leiomyosarcoma, pancreas cancer including ductal adenocarcinoma, insulinoma, glucagonoma, gastrinoma, carcinoid tumors, and vipoma, small bowel cancer including adenocarcinoma, lymphoma, carcinoid tumors, Kaposi's sarcoma, leiomyoma, hemangioma, lipoma, neurofibroma, and fibroma, large bowel cancer including adenocarcinoma, tubular adenoma, villous adenoma, hamartoma, and leiomyoma;
d) Genitourinary tract cancer consisting of: kidney including adenocarcinoma, Wilm's tumor, nephroblastoma, lymphoma, and leukemia, bladder and urethra including squamous cell carcinoma, transitional cell carcinoma, and adenocarcinoma, prostate including adenocarcinoma and sarcoma, testis including seminoma, teratoma, embryonal carcinoma, teratocarcinoma, choriocarcinoma, sarcoma, interstitial cell carcinoma, fibroma, fibroadenoma, adenomatoid tumors, and lipoma;
e) Liver cancer consisting of: hepatoma including hepatocellular and carcinoma, cholangiocarcinoma, hepatoblastoma, angiosarcoma, hepatocellular adenoma, hemangioma;
f) Biliary tract cancer consisting of: gall bladder carcinoma, ampullary carcinoma, cholangiocarcinoma;
g) Bone cancer consisting of: osteogenic sarcoma, osteosarcoma, fibrosarcoma, malignant fibrous histiocytoma, chondrosarcoma, Ewing's sarcoma, malignant lymphoma including reticulum cell and sarcoma, multiple myeloma, malignant giant cell tumor chordoma, osteochronfroma including osteocartilaginous and exostoses, benign chondroma, chondroblastoma, chondromyxofibroma, osteoid osteoma and giant cell tumors;
h) Nervous system cancer consisting of: skull including osteoma, hemangioma, granuloma, xanthoma, and osteitis deformans, meninges including meningioma, meningiosarcoma, and gliomatosis, brain including astrocytoma, medulloblastoma, glioma, ependymoma, germinoma, pinealoma, glioblastoma multiform, oligodendroglioma, schwannoma, retinoblastoma, and congenital tumors, spinal cord including neurofibroma, meningioma, glioma, and sarcoma;
i) Gynecological cancer consisting of: uterus including endometrial carcinoma, serous cystadenocarcinoma, mucinous cystadenocarcinoma, unclassified carcinoma, granulosa-thecal cell tumors, Sertoli-Leydig cell tumors, dysgerminoma, and malignant teratoma, vulva including squamous cell carcinoma, intraepithelial carcinoma, adenocarcinoma, fibrosarcoma, and melanoma, vagina including clear cell carcinoma, squamous cell carcinoma, botryoid sarcoma, embryonal rhabdomyosarcoma, fallopian tubecarcinoma;
j) Hematologic cancer consisting of: blood including acute myeloid leukemia chronic myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma, malignant lymphoma;
k) Skin cancer consisting of: malignant melanoma, basal cell carcinoma, squamous cell carcinoma, Kaposi's sarcoma, moles dysplastic nevi, lipoma, angioma, dermatofibroma, keloids, psoriasis; and
l) Adrenal gland cancer consisting of: neuroblastoma.

65. The method of claim 61, wherein the cancer is non-small cell lung cancer, small cell lung cancer, colorectal cancer, rectal cancer or pancreatic cancer.
